(19) United States
(12) Patent Application Publication
(10) Pub. No.: US 2025/0367411 A1
(43) Pub. Date: Dec. 4, 2025

(54) EXPANDABLE INTRODUCER

(71) Applicant: EDWARDS LIFESCIENCES CORPORATION, Irvine, CA (US)

(72) Inventors: Sovanpheap Mak, Santa Ana, CA (US); Ilan Tamir, Irvine, CA (US); Maxwell Harrison Fine, Costa Mesa, CA (US); Nasser William Saleh, Stockton, CA (US); Sonny Tran, Westminster, CA (US)

(21) Appl. No.: 19/301,722

(22) Filed: Aug. 15, 2025

Related U.S. Application Data
(63) Continuation of application No. PCT/US2024/016847, filed on Feb. 22, 2024.
(60) Provisional application No. 63/486,635, filed on Feb. 23, 2023.

(51) Int. Cl. A61M 25/06; A61F 2/24; A61M 25/00

(52) U.S. Cl. CPC A61M 25/0662; A61F 2/2427; A61M 25/0023; A61M 25/0043; A61M 25/0068; A61M 2025/0004; A61M 2025/0024; A61M 2025/0681; A61M 2210/12

(57) ABSTRACT

Various implementations include a sheath system and corresponding expandable introducer. The system includes an elongated introducer body and an elongated core member received within the central lumen of the introducer body. A portion of the introducer body expands in response to the outwardly directed radial force exerted on the central lumen of introducer body by the core member as it moves between the unexpanded and expanded positions.

### CROSS-REFERENCE TO RELATED APPLICATIONS

This application is a continuation of PCT Patent Application No. PCT/US2024/016847 filed on Feb. 22, 2024, which application claims the benefit of U.S. Provisional Application No. 63/486,635, filed Feb. 23, 2023, each of which application being incorporated herein in its entirety by this reference.

### FIELD

The present application is directed to a sheath for use with catheter-based technologies for repairing and/or replacing heart valves, as well as for delivering an implant, such as a prosthetic valve to a heart via the patient's vasculature.

### BACKGROUND

Endovascular delivery catheter assemblies are used to implant prosthetic devices, such as a prosthetic valve, at locations inside the body that are not readily accessible by surgery or where access without invasive surgery is desirable. For example, aortic, mitral, tricuspid, and/or pulmonary prosthetic valves can be delivered to a treatment site using minimally invasive surgical techniques.

Percutaneous interventional medical procedures utilize the large blood vessels of the body reach target destinations rather than surgically opening target site. There are many types of diseases states that can be treated via interventional methods including coronary blockages, valve replacements (TAVR) and brain aneurysms. These techniques involve using wires, catheters, balloons, electrodes and other thin devices to travel down the length of the blood vessels from the access site to the target site. The devices have a proximal end which the clinician controls outside of the body and a distal end inside the body which is responsible for treating the disease state. Percutaneous interventional procedures offer several advantages over open surgical techniques. First, they require smaller incision sites which reduces scarring and bleeding as well as infection risk. Procedures are also less traumatic to the tissue, so recovery times are reduced. Finally, interventional techniques can usually be performed much faster, and with fewer clinicians participating in the procedure, so overall costs are lowered. In some cases, the need for anesthesia is also eliminated, further speeding up the recovery process and reducing risk.

A single procedure typically uses several different guidewires, catheters, and balloons to achieve the desired effect. One at a time, each tool is inserted and then removed from the access site sequentially. For example, a guidewire is used to track the correct location within the body. Next a balloon may be used to dilate a section of narrowed blood vessel. Last, an implant may be delivered to the target site. Because catheters are frequently inserted and removed, introducer sheaths are used to protect the local anatomy and simplify the procedure.

An introducer sheath can be used to safely introduce a delivery apparatus into a patient's vasculature (for example, the femoral artery). Introducer sheaths are conduits that seal onto the access site blood vessel to reduce bleeding and trauma to the vessel caused by catheters with rough edges. An introducer sheath generally has an elongated sleeve that is inserted into the vasculature and a housing that contains one or more sealing valves that allow a delivery apparatus to be placed in fluid communication with the vasculature with minimal blood loss. Once the introducer sheath is positioned within the vasculature, the shaft of the delivery apparatus is advanced through the sheath and into the vasculature, carrying the prosthetic device. Expandable introducer sheaths, formed of highly elastomeric materials, allow for the dilating of the vessel to be performed by the passing prosthetic device.

The expandable sheath, formed of highly elastomeric materials and some including one or more folds to aid in expansion, expands as an implantable device is inserted through the sheath. These sheaths sometimes include a strain relief portion that extends along/over the outer surface of the sheath (for example, at the proximal end) and forms a smooth transition from the sheath hub to the sheath. The strain relief portion restricts expansion of the underlying sheath and helps to ensure hemostasis between the portions of the sheath inside the patient and the sheath hub (external to the patient). Because the strain relief portion resists expansion, higher push forces are required as the delivery device/system and implant are introduced into and advanced through the sheath/strain relief portion. In addition, recent trends in heart valves including thicker PVL skirts has increased the crimped profile of the heart valve/delivery device and can lead to even higher push forces through the sheath, and particularly the strain relief portion.

One method to reduce push forces required to advance the delivery device through the sheath is to pre-dilate the sheath and/or strain relief portion by passing a relatively large dilator (for example, 22 French dilator) into the sheath. This is done during sheath prep, prior to sheath insertion into the patient and/or with the sheath at least partially inserted into the patient. The challenge with this method is that it can be difficult with regard to physical strength of the user (i.e., grip and arm strength) to advance the dilator into sheath. Additionally, it is important that the dilator pass all the way to the distal end sheath while also avoiding splitting of the sheath and/or distal end of the sheath, which could cause difficulty or vessel injury during the delivery device insertion/removal process.

Accordingly, there remains a need for devices, systems, and methods of providing a sheath including a strain relief portion, that allows the sheath body and distal tip to expand reducing the initial push force when introducing the delivery system and implant.

### SUMMARY

Implementations of the present expandable sheath system can minimize trauma to the vessel and damage to the sheath and prosthetic device by reducing push forces through the sheath. Some implementations ensure that the sheath is not damaged during efforts to dilate or expand the strain relief portion. Some implementations can comprise a sheath with a smaller profile than that of prior art introducer sheaths. Furthermore, certain implementations can reduce the length of time a procedure takes, as well as reduce the risk of a longitudinal or radial vessel tear, or plaque dislodgement, because lower push force is required and only one sheath is used, rather than several different sizes of sheaths.

An implementation of the present disclosure is an expandable introducer system that includes: an elongated introducer body and an elongated core member received within the central lumen of the introducer body and movable between an unexpanded and an expanded position,

In some implementations, the elongated introducer body includes: a central lumen extending between a proximal and distal end of the introducer body; a slit extending axially from a distal end toward a proximal end of the introducer body; and a slotted opening extending axially along a portion of the introducer body. In some embodiments, the sheath system includes a distal introducer tip coupled to the distal end of the of the introducer body. At least a portion of the introducer body is configured to expand from an unexpanded configuration in which the introducer body has a first outer diameter to an expanded configuration in which the introducer body has a second, larger, outer diameter due to an outwardly directed radial force exerted on the central lumen of introducer body by the core member as it moves between the unexpanded and expanded positions.

In some implementations, the core member includes an elongated body portion and an expansion element extending radially from an outer surface of the body portion, where contact between the expansion element and the central lumen of the introducer body provides the outwardly directed radial force on the central lumen of the introducer body for directing expansion of the introducer body.

In some implementations, the central lumen of the introducer body includes a cavity for receiving the expansion element when the core member is in the unexpanded position, the cavity having a size and shape corresponding to the size and shape of the expansion element (for example, the cavity including a tapered proximal edge), the cavity having a diameter greater than a diameter of a main portion of the central lumen, where, when the core member is in the unexpanded position, the expansion element is received within the cavity and the introducer body is not expanded, and where, when the core member is moved to the expanded position, the core member is moved axially and the expansion element is moved from the cavity to the main portion of the central lumen, thereby causing the introducer body to expand.

In some implementations, a portion of the introducer body including the slotted opening defines a portion of the introducer body that is movable between the unexpanded and the expanded configuration, wherein the cavity is provided within the central lumen at a location corresponding to the slotted opening.

In some implementations, the introducer body includes a plurality of slits extending from the distal end toward the proximal end of the introducer body.

In some implementations, the plurality of slits are evenly spaced around the circumference of the introducer body (for example, 10 slits).

In some implementations, the slit extends at least partially through a wall thickness of the introducer body (for example, the slit is provided by a weakened portion, such as a longitudinal perforation, score line, and/or slit).

In some implementations, the slit extends through the entire wall thickness of the introducer body.

In some implementations, the slit is provided on a shoulder extending from the distal end of the introducer body, the shoulder having a smaller diameter than a main body portion of the introducer body (for example, the slit extends along a majority of the length of the recessed shoulder, the slit extends along the entire length of the recessed shoulder).

In some implementations, a width of the slit is constant along an entire length of the slit.

In some implementations, the introducer body includes a plurality slotted openings.

In some implementations, the plurality of slotted openings are evenly spaced around the circumference of the introducer body.

In some implementations, the introducer body includes ten slotted openings.

In some implementations, the slotted opening extends at least partially through an entire wall thickness of the introducer body (for example, the slotted opening is provided by a weakened portion, such as a longitudinal perforation, score line, and/or slit).

In some implementations, the slotted opening extends through the entire wall thickness of the introducer body.

In some implementations, the slotted opening extends along a portion of the introducer body between the proximal and distal ends (for example, the slotted opening is provided on the distal end, and does not extend to the distal end of the introducer body).

In some implementations, the introducer body further includes: a shoulder extending from the distal end of the introducer body toward a proximal end of the body, the shoulder having a smaller diameter than a main body portion of the introducer body, where the shoulder is sized and configured to be received within a central lumen of the distal introducer tip, where the slotted opening is provided on the main body portion of the introducer body.

In some implementations, a portion of the slotted opening extends along a portion of the shoulder.

In some implementations, the slit is provided on the shoulder extending from the distal end of the introducer body, where a portion of a length of the slit axially overlaps with a portion of a length of the slotted opening along the shoulder.

In some implementations, a width of the slotted opening tapers at a distal end of the slotted opening.

In some implementations, the introducer body further includes: a shoulder extending from the distal end of the introducer body toward a proximal end of the introducer body, the shoulder having a smaller diameter than a main body portion of the introducer body, where the shoulder is sized and configured to be received within a central lumen of the introducer tip.

In some implementations, the shoulder extends around a circumference of the introducer body and includes a body engagement feature extending circumferentially around the shoulder, where the body engagement feature is sized and configured to receive a corresponding tip engagement feature provided on the central lumen of the introducer tip.

In some implementations, the body engagement feature includes an annular recess extending circumferentially around the shoulder and the tip engagement feature includes a correspondingly shaped projection extending radially from the central lumen of the distal introducer tip.

In some implementations, the introducer tip includes a tapered distal end portion.

In some implementations, the tapered distal end portion tapers from a distal end of the introducer tip to a main body portion of the introducer tip (for example, the main body portion including a constant diameter portion extending from the tapered portion toward the proximal end, the tapered distal portion and the main body portion providing a smooth and tight fit between the introducer and the distal opening of the sheath).

In some implementations, the introducer tip includes a slit extending axially from a proximal end toward the distal end of the introducer tip.

In some implementations, the introducer tip includes a plurality of slits (for example, 5 slits).

In some implementations, the plurality of slits are evenly spaced around the circumference of the introducer tip.

In some implementations, the slit extends at least partially through a wall thickness of the introducer tip (for example, the slit is provided by a weakened portion, such as a longitudinal perforation, score line, and/or slit).

In some implementations, the slit extends through the entire wall thickness of the introducer tip.

In some implementations, the introducer tip includes an annular groove, where the introducer body is received within the central lumen of the introducer tip, and where the introducer tip is coupled to the introducer body by a retaining ring (for example, stainless steel retaining ring, continuous or discontinuous ring/clip) provided over the outer surface of the introducer tip within the annular groove such that radially inward force provided by the retaining ring compresses the introducer tip between the retaining ring and the introducer body.

In some implementations, the introducer assembly further includes a slot sleeve coupled to the proximal end of the introducer body, the slot sleeve including a guide slot for receiving a traveling pin provided on the core member, the traveling pin movable within the guide slot as the core member moves between an unexpanded and an expanded positions.

In some implementations, the slot sleeve is fixedly coupled to the proximal end of the introducer body, for example, by a mechanical fastener (for example, a press fit, an interference fit, a snap fit, a pin, thread, bayonet fastener, clip, locking key), a chemical fastener (for example, an adhesive, a UV bonded adhesive), a thermal process (for example, a weld, a reflow process), and/or any other suitable coupling process known in the art).

In some implementations, the guide slot is generally L-shaped, including an axially-extending proximal portion and a circumferentially-extending distal portion.

In some implementations, the guide slot includes a catch for maintaining the traveling pin in the expanded position.

In some implementations, the expansion element includes a regular or irregular shaped projection extending from the outer surface of the body portion (for example, in some aspects, the expansion element can extend around all or a portion of the circumference of the shaft).

In some implementations, the expansion element has a circular shape in cross-section.

In some implementations, a diameter of the expansion element is approximately 22 F (for example, the expansion element of dilator has a diameter ranging from 12 Fr to 22 Fr, from 14 Fr to 22 Fr, in some aspects, the expansion element has a diameter of 22 Fr (7.3 mm, 0.288 inches), the main portion of the central lumen when expanded by the expansion element is approximately 24 Fr).

In some implementations, the core member is received and movable within at least a portion of a central lumen of the introducer tip.

In some implementations, the core member includes a central lumen extending therethrough.

In some implementations, the core member includes a tapered distal end.

In some implementations, a proximal end of the core member is coupled to a core member hub (for example, where the core member hub includes a luer hub).

In some implementations, the traveling pin is provided on the core member hub, the traveling pin extending radially from the core member hub (for example, stainless steel pin).

In some implementations, the core member hub includes a hub shoulder extending from a distal end of the core member hub toward a proximal end of the core member hub, the hub shoulder having a smaller diameter than a main body portion of the core member hub, where the hub shoulder is sized and configured to be received within a central lumen of the slot sleeve.

In some implementations, the core member hub includes a gripping feature including one or more opposing depressions/recesses.

In some implementations, the introducer assembly further includes a connecting sleeve including a sleeve body having a proximal end and a distal end and defining a central lumen extending longitudinally between the proximal end and the distal end, where the distal end of the slot sleeve is received within the central lumen of the connecting sleeve, where the connecting sleeve includes a connecting sleeve guide slot for receiving a coupling pin provided on the slot sleeve, the coupling pin extending radially from an outer surface of the slot sleeve, the coupling pin received and movable within the connecting sleeve guide slot for moving the slot sleeve between an unexpanded and an expanded position and for coupling the introducer assembly to a sheath assembly.

In some implementations, the coupling pin is coupled to the slot sleeve by a spring support such that the coupling pin moves radially (for example, in and out) allowing the slot sleeve to be coupled to the connecting sleeve.

In some implementations, the connecting sleeve guide slot is generally L-shaped, including a circumferentially-extending proximal portion and an axially-extending distal portion.

In some implementations, the connecting sleeve is coupled to a sheath hub, the sheath hub having an elongated body portion with a central lumen extending therethrough and an expandable sheath coupled to a distal end of the body portion, where a central lumen of the expandable sheath is aligned with the central lumens of the sheath hub and the connecting sleeve.

In some implementations, the expandable sheath includes a continuous inner layer defining a lumen therethrough, the inner layer having at least one folded portion.

In some implementations, the introducer body is formed form a stiffer material than the introducer tip (for example, the introducer body is constructed from a stiffer material to allow for manufacturing of the slotted openings, which are too difficult to manufacture in a soft material).

In some implementations, the introducer body is formed from polyethylene (for example, a material comprising low-density polyethylene (LDPE) 60%, high performance polyethylene (HPPE) 20%, and barium sulfate (BaSO4) 20%).

In some implementations, the introducer tip is formed from polyethylene (for example, a material comprising low-density polyethylene (LDPE) 70%, high performance polyethylene (HPPE) 10%, and barium sulfate (BaSO4) 20%).

In some implementations, the core member is formed from a polymeric material (for example, polyethylene (LDPE) 60%, high performance polyethylene (HPPE) 20%, and barium sulfate (BaSO4) 20%).

In some implementations, the slot sleeve is formed from a polycarbonate (for example, an overmolded polycarbonate).

A further implementation of the present disclosure provides a method of expanding a sheath comprising: advancing the elongated core member into the central lumen of an introducer body, wherein at least a portion of the introducer body is configured to expand from an unexpanded configuration in which the introducer body has a first outer diameter to an expanded configuration in which the introducer body has a second, larger, outer diameter due to an outwardly directed radial force exerted on the central lumen of introducer body by the core member as it moves between an unexpanded and expanded position within the central lumen; coupling the core member to the introducer body; positioning the core member in the unexpanded (home) position within the central lumen of the introducer body; advancing the coupled core member and introducer body into the central lumen of an expandable sheath; coupling the introducer body to the sheath (for example, via a slot sleeve coupled to a proximal end of the introducer body); positioning the introducer body (for example, coupled introducer body and sleeve) in an unexpanded (home) position with respect to the sheath; and advancing the core member within the central lumen of the introducer body such that the core member moves from the unexpanded (home) position to the expanded position where the core member exerts an outwardly directed radial force on the central lumen expanding the introducer body and a corresponding portion of the sheath.

In some implementations, advancing the core member to the expanded position includes advancing the core member distally within the introducer body.

In some implementations, advancing the core member to the expanded position includes advancing a traveling pin extending radially from the core member within a guide slot, provided on a slot sleeve coupled to a proximal end of the introducer body, to a traveling pin expanded position (for example, advancing the traveling pin distally along the axially-extending proximal portion of the guide slot and rotationally within the circumferentially-extending distal portion of the guide slot).

In some implementations, the core member includes an elongated body portion and an expansion element extending radially from an outer surface of the body portion, where, when the core member is in the expanded position, contact between the expansion element and the central lumen of the introducer body provides the outwardly directed radial force on the central lumen of the introducer body directing expansion of the introducer body.

In some implementations, the central lumen of the introducer body includes a cavity for receiving the expansion element when the core member is in the unexpanded position, the cavity having a size and shape corresponding to the size and shape of the expansion element (for example, the cavity including a tapered proximal edge), the cavity having a diameter greater than a diameter of a main portion of the central lumen, where positioning the core member in the unexpanded (home) position within the central lumen of the introducer body includes positioning the expansion element within the cavity such that the introducer body is not expanded, where advancing the core member to the expanded position within the central lumen includes moving the core member such that the expansion element moves from the cavity and into the (smaller diameter) main portion of the central lumen causing the introducer body to expand.

In some implementations, the method further includes expanding a distal opening of the sheath by positioning the core member in the expanded position and advancing the core member and the introducer body within the central lumen of the sheath such that an expanded portion of the introducer body is advanced through a distal opening of the sheath, where the core member/introducer body exert an outwardly directed radial force expanding the distal opening (for example, to a profile/diameter corresponding to the combined introducer body).

In some implementations, the core member and introducer body are coupled together (for example, axially and/or rotationally fixed) before the combined core member and introducer body is advanced through the distal opening of the sheath.

In some implementations, expanding the distal opening further includes: coupling the core member to the introducer body by engaging a traveling pin extending radially from the core member within a guide slot provided on a sleeve coupled to a proximal end of the introducer body; coupling the introducer body to the sheath by coupling the slot sleeve to a sheath connecting sleeve, engaging a coupling pin extending radially from the slot sleeve within a connecting sleeve guide slot provided on the sheath connecting sleeve, where the sheath connecting sleeve is coupled to a proximal end of the expandable sheath; advancing the core member to the expanded position by advancing the traveling pin within the guide slot to a traveling pin expanded position (for example, advancing the traveling pin distally along the axially-extending proximal portion of the guide slot and rotationally within the circumferentially-extending distal portion of the guide slot); and advancing the slot sleeve within the connecting sleeve to the expanded position (for example, distal tip expanded configuration) by advancing the coupling pin with the connecting sleeve guide slot to a coupling pin expanded position (for example, advancing the coupling pin rotationally within the circumferentially-extending proximal portion of the connecting sleeve guide slot and distally along the axially-extending distal portion of the connecting sleeve guide slot), thereby moving the combined introducer body and core member (in the expanded position) through the distal opening of the sheath.

In some implementations, the method further includes withdrawing the expanded introducer body through the sheath thereby expanding the sheath by: withdrawing the slot sleeve from the connecting sleeve by withdrawing (proximally) the coupling pin within the connecting sleeve guide slot to a coupling pin unexpanded (home) position (for example, withdrawing the coupling pin proximally along the axially-extending distal portion of the connecting sleeve guide slot and rotationally within the circumferentially-extending proximal portion of the connecting sleeve guide slot), thereby moving the combined introducer body and core member (in the expanded position) proximally through the distal opening of the sheath; depressing the coupling pin such that the slot sleeve and the coupling pin are movable (for example, rotationally and/or axially) within the central lumen of the connecting sleeve (for example, depressing the coupling pin to have a height corresponding to the outer surface of the slot sleeve such that the depressed coupling pin is axially and rotationally movable withing the central lumen of the connecting sleeve); removing the slot sleeve from the connecting sleeve; and withdrawing the introducer body from the central lumen of the sheath.

In some implementations, coupling the core member to the introducer body includes engaging a traveling pin extending radially from the core member within a guide slot provided on a slot sleeve coupled to a proximal end of the introducer body.

In some implementations, the traveling pin of the core member is radially movable (for example, in and out) with respect an outer surface of the core member (and/or core member hub) and includes a spring support biasing the traveling pin in an outward position. In some implementations, coupling the core member to the introducer body includes: depressing the traveling pin such that the core member (and/or core member hub) and traveling pin are movable (for example, rotationally and/or axially) within a central lumen of the slot sleeve (for example, depressing the traveling pin to have a height corresponding to the outer surface of the core member hub such that the depressed traveling pin is axially and rotationally movable withing the central lumen of the slot sleeve); and moving the core member and/or core member hub rotationally and/or axially until the traveling pin is received within the guide slot and the traveling pin is released from its depressed condition.

In some implementations, the core member is secured to the slot sleeve in the unexpanded (home) position by a sleeve spring provided between a distal end of the core member hub and an inner shoulder provided on the central lumen of the slot sleeve (for example, the slot sleeve spring biases the core member proximally, keeping the expansion element is the home/unexpanded position) (for example, the core member is pushed distally, moving the traveling pin within the (slot) toward the (active/expanded) position).

In some implementations, positioning the core member in the unexpanded (home) position within the central lumen of the introducer body includes positioning the traveling pin located at an unexpanded (home) position within the guide slot.

In some implementations, positioning the introducer body (for example, coupled introducer body and sleeve) in an unexpanded (home) position with respect to the sheath (for example, with respect to the sheath connecting sleeve) includes: positioning the coupling pin at an unexpanded (home) position within the connecting sleeve guide slot.

In some implementations, coupling the introducer body to the sheath includes coupling the introducer body to the sheath via a slot sleeve coupled to a proximal end of the introducer body.

In some implementations, coupling the introducer body to the sheath includes coupling the slot sleeve to a sheath connecting sleeve by engaging a coupling pin extending radially from the slot sleeve within a connecting sleeve guide slot provided on the sheath connecting sleeve, where the sheath connecting sleeve is coupled to a proximal end of the expandable sheath.

In some implementations, the coupling pin is radially movable (for example, in and out) with respect the outer surface of the slot sleeve and includes a spring support biasing the coupling pin in an outward position. In some implementations, coupling the slot sleeve to the sheath connecting sleeve includes: depressing the coupling pin such that the slot sleeve and coupling pin are movable (for example, rotationally and axially) within a central lumen of the sheath connecting sleeve (for example, depressing the coupling pin to have a height corresponding to the outer surface of the slot sleeve such that the depressed coupling pin is axially and rotationally movable withing the central lumen of the sheath connecting sleeve); and moving the slot sleeve rotationally and/or axially until the coupling pin is received within the connecting sleeve guide slot and the coupling pin is released from its depressed condition.

In some implementations, the slot sleeve is secured in the unexpanded (home) position by engagement between the coupling pin and the connecting sleeve guide slot (for example, the coupling pin is retained in the proximal most portion (for example, the circumferentially-extending proximal portion of the connecting sleeve guide slot). In some examples, the connecting sleeve guide slot includes a catch for maintaining the coupling pin in the unexpanded (home) position.

In some implementations, the method further includes coupling the connecting sleeve to a sheath hub, where the sheath hub is fixedly (for example, axially and rotationally fixed) coupled to the sheath.

Another implementation of the present disclosure provides a method of delivering a medical device through a sheath, the method including: providing a radially expandable sheath including a continuous inner layer defining a central lumen therethrough, the inner layer having at least one folded portion extending along a length of the inner layer; advancing the elongated core member into the central lumen of an introducer body, wherein at least a portion of the introducer body is configured to expand from an unexpanded configuration in which the introducer body has a first outer diameter to an expanded configuration in which the introducer body has a second, larger, outer diameter due to an outwardly directed radial force exerted on the central lumen of introducer body by the core member as it moves between an unexpanded and expanded position within the central lumen; coupling the core member to the introducer body; positioning the core member in the unexpanded (home) position within the central lumen of the introducer body; advancing the coupled core member and introducer body into the central lumen of the sheath expandable sheath; coupling the introducer body to the sheath (for example, via a sleeve coupled to a proximal end of the introducer body); positioning the introducer body (for example, coupled introducer body and sleeve) in an unexpanded (home) position with respect to the sheath; inserting the introduce body and the sheath at least partially into the blood vessel of the patient; advancing the core member within the central lumen of the introducer body such that the core member moves from the unexpanded (home) position to the expanded position where the core member exerts an outwardly directed radial force on the central lumen expanding the introducer body and a corresponding portion of the sheath; moving the introducer body within the central lumen of the sheath causing the sheath to locally expand from the unexpanded configuration to the expanded configuration at a location proximate the medical device in response to the outwardly directed radial force of the introducer body exerted against the inner layer and locally contracting the sheath at least partially back to the unexpanded configuration as (the expanded portion of) the introducer body passes through the lumen; withdrawing the introducer body from the sheath; and advancing a medical device through the central lumen of the sheath and beyond the distal opening of the sheath to the treatment site.

In some implementations, the inner layer includes at least one folded portion, wherein locally expanding the lumen of the sheath causes a length of the folded portion to at least partially unfold.

In some implementations, the sheath further includes: an outer layer provided over the inner layer, where the outer layer is discontinuous and includes an overlapping portion and an underlying portion, where when the sheath is in the unexpanded configuration, the overlapping portion overlaps the underlying portion with the folded portion of the inner layer disposed between the overlapping portion and the underlying portion, and where the strain relief layer extends at least partially over the outer layer.

In some implementations, the medical device is a prosthetic device mounted in a radially crimped state on a delivery apparatus.

In some implementations, the prosthetic device comprises a prosthetic heart valve and the method further comprises implanting the prosthetic heart valve at a treatment site within the patient.

In some implementations, the prosthetic heart valve is mounted on a balloon catheter of a delivery apparatus as the prosthetic heart valve is advanced through the sheath.

In some implementations, the sheath is inserted into a femoral artery of the patient.

Various aspects of the implementations described herein can be combined based on desired sheath system characteristics.

### DESCRIPTION OF DRAWINGS

FIG. 1 is an elevation view of an expandable sheath along with an endovascular delivery apparatus for implanting a prosthetic implant.

FIG. 2 is an elevation view of an expandable sheath including an introducer locking hub, a sheath locking sleeve, and an introducer.

FIG. 3 is an elevation view of the expandable sheath of FIG. 2 along with an endovascular delivery apparatus for implanting a prosthetic implant.

FIG. 4 is an elevation view of an expandable sheath a sheath hub, an introducer locking hub, and a sheath locking sleeve of FIG. 2.

FIG. 5A is a cross sectional view of the sheath hub, introducer locking hub, and sheath locking sleeve of FIG. 2.

FIG. 5B is a cross sectional view of the introducer cap, the sheath hub, the introducer locking hub, the sheath locking sleeve of FIG. 2.

FIG. 6 is a cross sectional view of the introducer cap, sheath hub, introducer locking hub, and sheath locking sleeve of FIG. 2.

FIG. 7 is a distal end view of the sheath locking sleeve of FIG. 2 and the proximal fluid seal of FIGS. 5A-B.

FIG. 8A is a first elevation view of the introducer locking hub of FIG. 2 coupled to an introducer.

FIG. 8B is a second (rotated) elevation view of the introducer locking hub of FIG. 2 coupled to the introducer.

FIG. 8C is a distal end view of the introducer locking hub of FIG. 2 coupled to the introducer.

FIG. 8D is a partial side view of the introducer locking hub of FIG. 2 coupled to the introducer.

FIG. 8E is a partial perspective view of the introducer locking hub of FIG. 2 coupled to the introducer.

FIG. 8F is a partial perspective view of the introducer locking hub of FIG. 2 coupled to the introducer.

FIG. 9A is a distal end view of the introducer locking hub of FIG. 2.

FIG. 9B is a first elevation view of the introducer locking hub of FIG. 2.

FIG. 9C is a proximal end view of the introducer locking hub of FIG. 2.

FIG. 9D is a first perspective view of the introducer locking hub of FIG. 2.

FIG. 9E is a second elevation view of the introducer locking hub of FIG. 2.

FIG. 9F is a second perspective view of the introducer locking hub of FIG. 2.

FIG. 10A is a distal end view of the sheath locking sleeve of FIG. 2.

FIG. 10B is a first elevation view of the sheath locking sleeve of FIG. 2.

FIG. 10C is a proximal end view of the sheath locking sleeve of FIG. 2.

FIG. 10D is a first perspective view of the sheath locking sleeve of FIG. 2.

FIG. 10E is a second elevation view of the sheath locking sleeve of FIG. 2.

FIG. 10F is a second perspective view of the sheath locking sleeve of FIG. 2.

FIG. 11 is a side elevation cross-sectional view of a portion of the expandable sheath of FIGS. 1 and 2.

FIG. 12 is an enlarged view of a portion of the expandable sheath of FIGS. 1 and 2.

FIG. 13A is an enlarged view of a portion of the expandable sheath of FIGS. 1 and 2 with the outer layer removed for purposes of illustration.

FIG. 13B is an enlarged view of a portion of the braided layer of the sheath of FIGS. 1 and 2.

FIG. 14 is an enlarged view of a portion of the expandable sheath of FIGS. 1 and 2 illustrating expansion of the sheath as a prosthetic device is advanced through the sheath.

FIG. 15 is a side view of the expandable sheath of FIGS. 1 and 2.

FIG. 16 is an enlarged section view of the sheath of FIG. 15 along section line 16-16.

FIG. 17 is cross-sectional view of the unexpanded sheath of FIG. 16 along section line 17-17.

FIG. 18 is cross-sectional view of the unexpanded sheath of FIG. 15 along section line 18-18.

FIG. 19 is cross-sectional view of the unexpanded sheath of FIG. 15 along section line 19-19.

FIG. 20 is cross-sectional view of the expanded sheath of FIG. 15 along section line 19-19.

FIG. 21 is a side view of the expandable sheath of FIGS. 1 and 2.

FIG. 22 is a cross-sectional view of the unexpanded sheath of FIG. 21 along section line 22-22.

FIG. 23 is a cross-sectional view of the expanded sheath of FIG. 21 along section line 22-22.

FIG. 24 is a side view of the expandable introducer system.

FIG. 25 is a side view of the elongated introducer body and slot sleeve of FIG. 24.

FIG. 26 is a side perspective view of the elongated introducer body and slot sleeve of FIG. 24.

FIG. 27 is a distal end view of the elongated introducer body of FIG. 24.

FIG. 28 is a side view of the elongated introducer body of FIG. 27.

FIG. 29 is a proximal end view of the elongated introducer body of FIG. 27.

FIG. 30 is a partial cross-sectional view of the elongated introducer body in the unexpanded configuration taken along section line 30-30 in FIG. 28.

FIG. 31A is a partial cross-sectional view of the elongated introducer body and core member in the unexpanded configuration taken along section line 30-30 in FIG. 28.

FIG. 31B is a partial cross-sectional view of the elongated introducer body and core member in the expanded configuration taken along section line 30-30 in FIG. 28.

FIG. 32 is a cross-sectional view of the elongated in introducer body in the unexpanded configuration taken along section line 32-32 in FIG. 31A.

FIG. 33 is a cross-sectional view of the elongated in introducer body in the expanded configuration taken along section line 33-33 in FIG. 31B.

FIG. 34 is a side view of the introducer tip and retaining ring of FIG. 24.

FIG. 35 is a side view of the introducer tip and retaining ring of FIG. 34.

FIG. 36 is a cross-sectional view of the introducer tip taken along section line 36-36 in FIG. 34.

FIG. 37 is a proximal end view of the introducer tip of FIG. 34.

FIG. 38 is a side view the retaining ring of FIG. 24.

FIG. 39 is an end view of the retaining ring of FIG. 38.

FIG. 40 is a perspective view of the retaining ring of FIG. 38.

FIG. 41 is a side view of the introducer body and sleeve of FIG. 24.

FIG. 42 is a distal end view of the slot sleeve of FIG. 41.

FIG. 43 is a side view of the slot sleeve of FIG. 41.

FIG. 44 is a proximal end view of the slot sleeve of FIG. 41.

FIG. 45 is a partial cross-sectional view of the introducer body and sleeve taken along section line 45-45 in FIG. 41.

FIG. 46 is a perspective view of the slot sleeve of FIG. 41.

FIG. 47 is a perspective view of the slot sleeve of FIG. 41.

FIG. 48 is a partial perspective view of the introducer system of FIG. 24.

FIG. 49 is a side view of the core member of FIG. 24.

FIG. 50 is a section view of the core member taken along section line 50-50 in

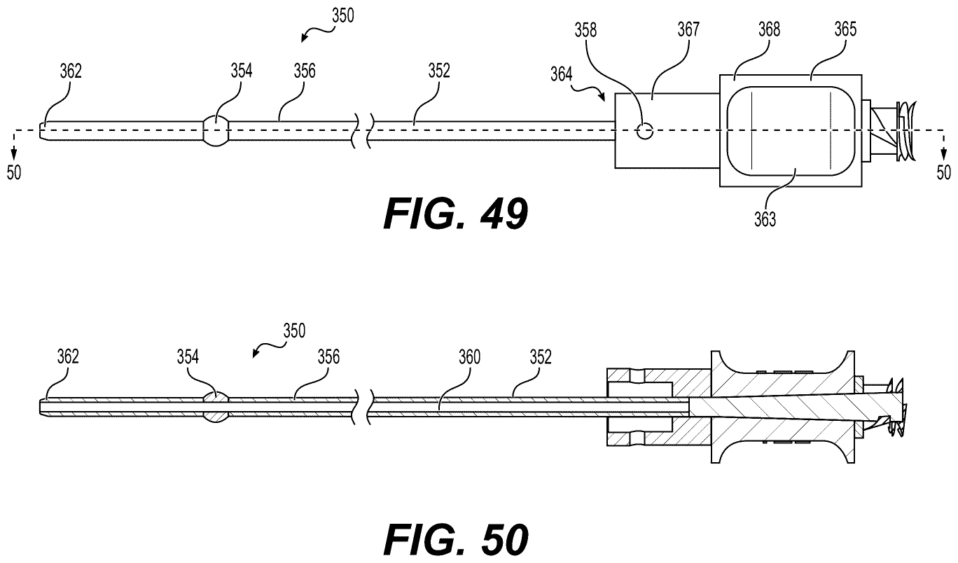

FIG. 49.

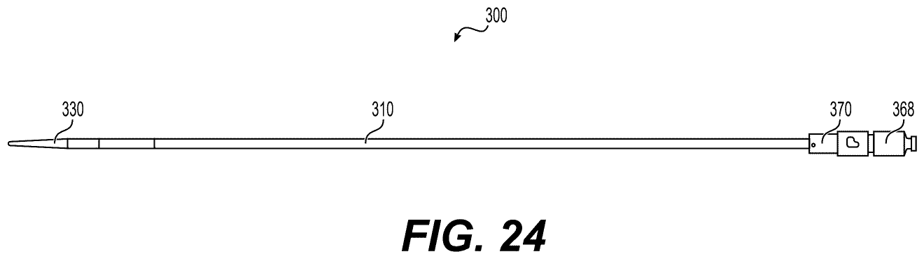

FIG. 51 is a side view of the expandable introducer system of FIG. 24 coupled to an expandable sheath and sheath hub by a connecting sleeve.

FIG. 52 is a distal end view of the connecting sleeve of FIG. 51.

FIG. 53 is a first side view of the connecting sleeve of FIG. 51.

FIG. 54 is a proximal end view of the connecting sleeve of FIG. 51.

FIG. 55 is a perspective view of the connecting sleeve of FIG. 51.

FIG. 56 is a second side view of the connecting sleeve of FIG. 51.

FIG. 57 is a perspective view of the connecting sleeve of FIG. 51.

FIG. 58 is a cross-sectional view of the connecting sleeve taken along line 58-58 in FIG. 53.

FIG. 59 is a side view of the introducer system and sheath.

FIG. 60 is an enlarged cross-sectional view of the introducer system and sheath taken along section line 60-60 in FIG. 59.

FIG. 61 is a partial side view of the distal end of the introducer system and sheath in the expanded configuration.

FIG. 62 is a partial side view of the introducer system and sheath in the expanded configuration.

FIG. 63 is a partial side view of the proximal end of the introducer system and sheath in the configuration where the slot sleeve is in the unexpanded position and the core member is in the expanded position.

FIG. 64 is a partial side view of the distal end of the introducer system and sheath in the expanded configuration.

FIG. 65 is a partial side cross-sectional view of the introducer system and sheath in the expanded configuration.

FIG. 66 is a partial side view of the proximal end of the introducer system and sheath in the expanded configuration.

FIG. 67 is a side view of the introducer system and sheath in the expanded configuration where the distal opening of the sheath is expanded.

FIG. 68 is an enlarged view of the expandable introducer system and expandable sheath of FIG. 67 taken along section 68-68.

FIG. 69 is a side view of the proximal end of the introducer system and sheath in the expanded configuration.

FIG. 70 is a side view of the proximal end of the introducer system and sheath in the unexpanded configuration where the slot sleeve is in the unexpanded position and the core member is in the expanded position.

FIG. 71 is a side view of the proximal end of the introducer system and sheath in the unexpanded configuration where the slot sleeve is removed from the connecting sleeve.

### DETAILED DESCRIPTION

The following description of certain examples of the inventive concepts should not be used to limit the scope of the claims. Other examples, features, aspects, implementations, and advantages will become apparent to those skilled in the art from the following description. As will be realized, the device and/or methods are capable of other different and obvious aspects, all without departing from the spirit of the inventive concepts. Accordingly, the drawings and descriptions should be regarded as illustrative in nature and not restrictive.

For purposes of this description, certain aspects, advantages, and novel features of the aspects of this disclosure are described herein. The described methods, systems, and apparatus should not be construed as limiting in any way. Instead, the present disclosure is directed toward all novel and nonobvious features and aspects of the various disclosed aspects, alone and in various combinations and sub-combinations with one another. The disclosed methods, systems, and apparatus are not limited to any specific aspect, feature, or combination thereof, nor do the disclosed methods, systems, and apparatus require that any one or more specific advantages be present or problems be solved.

Features, integers, characteristics, compounds, chemical moieties, or groups described in conjunction with a particular aspect or example of the present disclosure are to be understood to be applicable to any other aspect or example described herein unless incompatible therewith. All of the features disclosed in this specification (including any accompanying claims, abstract, and drawings), and/or all of the steps of any method or process so disclosed, may be combined in any combination, except combinations where at least some of such features and/or steps are mutually exclusive. The present disclosure is not restricted to the details of any foregoing aspects. The present disclosure extends to any novel one, or any novel combination, of the features disclosed in this specification (including any accompanying claims, abstract, and drawings), or to any novel one, or any novel combination, of the steps of any method or process so disclosed.

It should be appreciated that any patent, publication, or other disclosure material, in whole or in part, that is said to be incorporated by reference herein is incorporated herein only to the extent that the incorporated material does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

As used in the specification and the appended claims, the singular forms “a,” “an” and “the” include plural referents unless the context clearly dictates otherwise. Ranges may be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint.

“Optional” or “optionally” means that the subsequently described event or circumstance may or may not occur, and that the description includes instances where said event or circumstance occurs and instances where it does not.

The terms “proximal” and “distal” as used herein refer to regions of a sheath, catheter, or delivery assembly. “Proximal” means that region closest to handle of the device, while “distal” means that region farthest away from the handle of the device.

“Axially” or “axial” as used herein refers to a direction along the longitudinal axis of the sheath.

Throughout the description and claims of this specification, the word “comprise” and variations of the word, such as “comprising” and “comprises,” means “including but not limited to,” and is not intended to exclude, for example, other additives, components, integers or steps. “Exemplary” means “an example of” and is not intended to convey an indication of a preferred or ideal aspect. “Such as” is not used in a restrictive sense, but for explanatory purposes.

Expandable introducer sheaths are disclosed in U.S. Pat. No. 8,690,936, entitled “Expandable Sheath for Introducing an Endovascular Delivery Device into a Body,” U.S. Pat. No. 8,790,387, entitled “Expandable Sheath for Introducing an Endovascular Delivery Device into a Body,” U.S. Pat. No. 10,639,152, entitled “Expandable Sheath and Methods of Using the Same,” U.S. Pat. No. 10,792,471, entitled “Expandable Sheath,” U.S. Patent No. application Ser. No. 16/407,057, entitled “Expandable Sheath with Elastomeric Cross Sectional Portions,” U.S. Pat. No. 10,327,896, entitled “Expandable Sheath with Elastomeric Cross Sectional Portions,” U.S. Pat. No. 11,273,062, entitled “Expandable Sheath,” Application No. PCT/US2021/019514, entitled “Expandable sheath for introducing an endovascular delivery device in to a body,” Application No. PCT/US2021/031227, entitled “Expandable sheath for introducing an endovascular delivery device into a body,” Application No. PCT/US2021/031275, entitled “Expandable sheath for introducing an endovascular delivery device into a body,” U.S. application Ser. No. 17/113,268, entitled “Expandable Sheath and Method of Using the Same,” Application No. PCT/US2021/058247, entitled “Self-Expanding, Two Component Sheath,” Application No. PCT/US2022/012785, entitled “Expandable Sheath,” U.S. Pat. No. 11,051,939, entitled “Active Introducer Sheath System,” Application No. PCT/US2022/012684, entitled “Introducer with Sheath Tip Expander,” U.S. application Ser. No. 17/078,556, entitled “Advanced Sheath Patterns,” Application No. PCT/US2021/025038, entitled “Low temperature hydrophilic adhesive for use in expandable sheath for introducing an endovascular delivery device into a body,” Application No. PCT/US2021/050006, entitled “Expandable Sheath Including Reversable Bayonet Locking Hub,” U.S. Provisional Application No. 63/280,251, entitled “Expandable Sheath Gasket to Provide Hemostasis,” the disclosures of which are herein incorporated by reference.

Disclosed herein are elongate introducer sheaths that are particularly suitable for delivery of implants in the form of implantable heart valves, such as balloon-expandable implantable heart valves. Balloon-expandable implantable heart valves are well-known and will not be described in detail here. An example of such an implantable heart valve is described in U.S. Pat. No. 5,411,552, and also in U.S. Pat. No. 9,393,110, both of which are hereby incorporated by reference. The expandable introducer sheaths disclosed herein may also be used to deliver other types of implantable medical device, such as self-expanding and mechanically expanding implantable heart valves, stents or filters. Beyond transcatheter heart valves, the introducer sheath system can be useful for other types of minimally invasive surgery, such as any surgery requiring introduction of an apparatus into a subject's vessel. For example, the introducer sheath system can be used to introduce other types of delivery apparatus for placing various types of intraluminal devices (for example, stents, stented grafts, balloon catheters for angioplasty procedures, etc.) into many types of vascular and non-vascular body lumens (for example, veins, arteries, esophagus, ducts of the biliary tree, intestine, urethra, fallopian tube, other endocrine or exocrine ducts, etc.). The term “implantable” as used herein is broadly defined to mean anything-prosthetic or not—that is delivered to a site within a body. A diagnostic device, for example, may be an implantable.

Disclosed aspects of the expandable sheath system described herein can minimize trauma to the vessel by allowing for temporary expansion of a portion of the introducer sheath to accommodate the delivery system, followed by a return to the original diameter once the device passes through. Disclosed aspects of the introducer sheath prevent the introducer from separating from the sheath during insertion by locking the proximal hub of the introducer to the proximal hub of the sheath. Fixing the introducer and the sheath prevents the introducer from moving backward during insertion, thereby maintaining a snug fit and smooth transition between the introducer and the distal end of the sheath. Furthermore, present aspects can reduce the length of time a procedure takes, as well as reduce the risk of a longitudinal or radial vessel tear, or plaque dislodgement because only one sheath is required, rather than several different sizes of sheaths. Aspects of the present expandable sheath can avoid the need for multiple insertions for the dilation of the vessel.

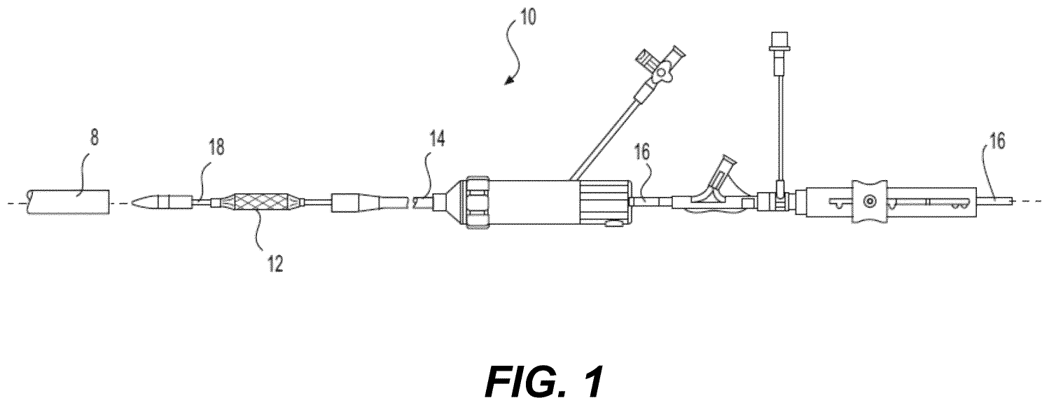

FIG. 1 illustrates an exemplary sheath 8 in use with a representative delivery apparatus 10, for delivering an implant 12, or other type of implantable (for example, tissue heart valve), to a patient. The delivery apparatus 10 can include a steerable guide catheter 14 (also referred to as a flex catheter) and a balloon catheter 16 extending through the guide catheter 14, and a nose catheter 15 extending through the balloon catheter 16. The guide catheter 14, balloon catheter 16, and nose catheter 15 in the illustrated example are adapted to slide longitudinally relative to each other to facilitate delivery and positioning of the implant 12 at an implantation site in a patient's body as described in detail herein. It is contemplated that the sheath 8 can be used with any type of elongated delivery apparatus used for implanting balloon-expandable prosthetic valves, self-expanding prosthetic valves, and other prosthetic devices.

As described in more detail herein, in general, the sheath 8 comprises an elongate expandable tube that, in use, is inserted into a vessel (for example, transfemoral vessel, femoral artery, iliac artery) by passing through the skin of patient, such that the distal end of the sheath 8 is inserted into the vessel. Sheath 8 includes a hemostasis valve and/or sealing features at the proximal end of the sheath, for example, in the sheath hub 20, that provide hemostasis and prevents blood leakage from the patient through the sheath 8. The sheath 8, including an introducer 6, is advanced into the patient's vasculature. Once positioned the introducer 6 is removed and the delivery apparatus 10 is inserted into/through the sheath 8, and the prosthetic device (implant 12) then be delivered and implanted within patient.

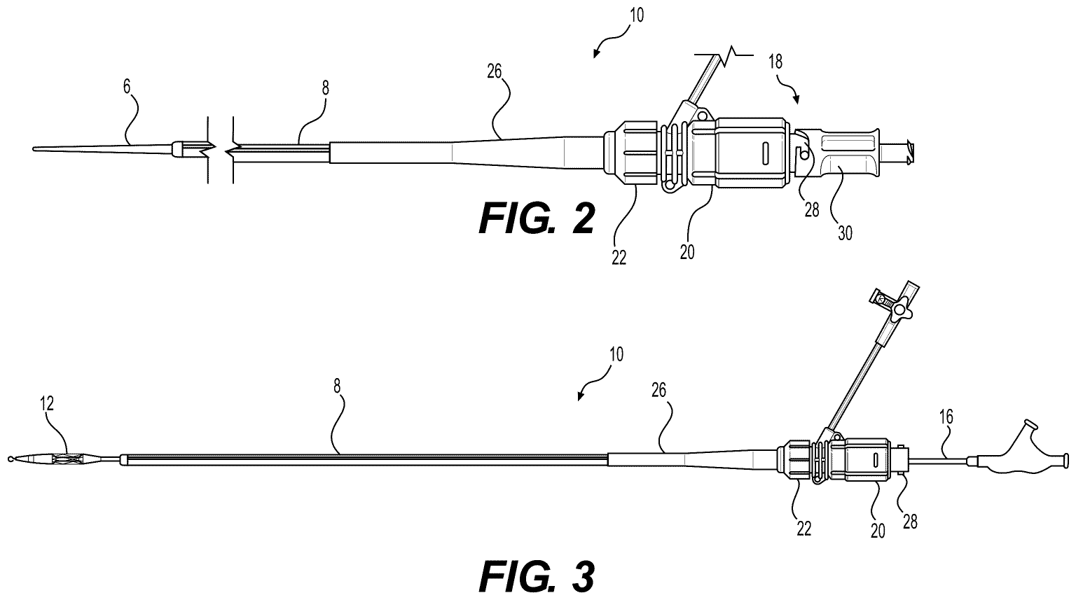

FIGS. 2 and 3, the introducer device/sheath assembly includes a sheath hub 20 at a proximal end of the device and an expandable sheath 8 extending distally from the sheath hub 20. The sheath 8 is coupled to the sheath hub 20 which in turn is removably coupled to a sheath locking system 18. The sheath locking system 18 allows the introducer 6, or other device desired to be removably couped (axially and rotatably) to the sheath 8.

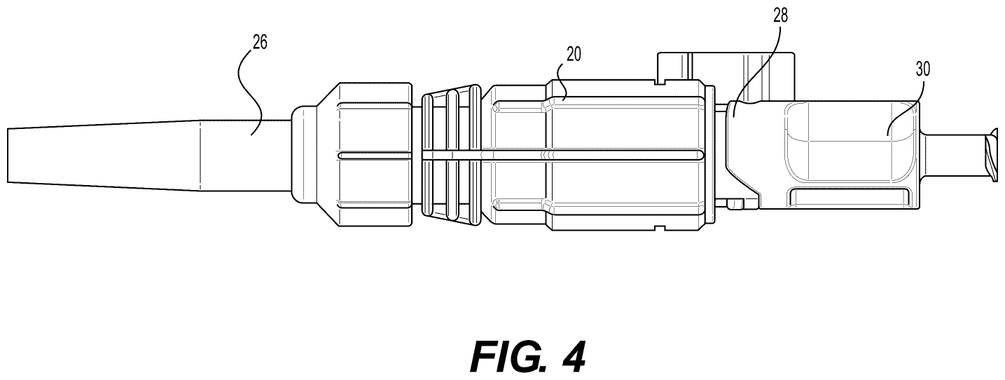

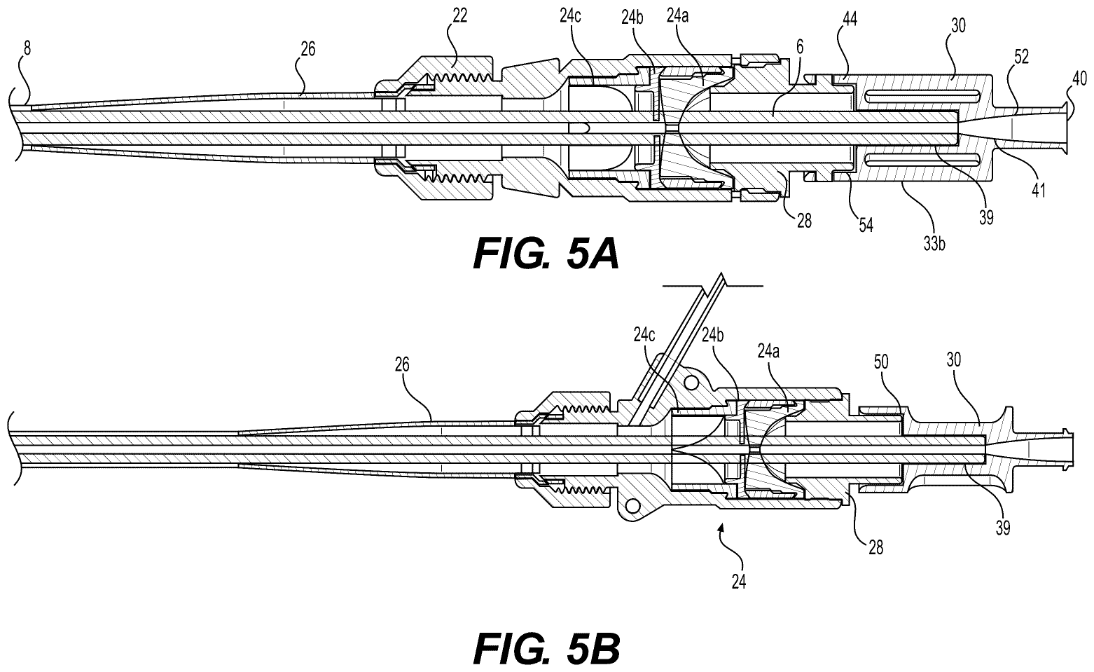

As illustrated in FIGS. 2-6, the sheath hub 20 can function as a handle for the device. The sheath hub 20 also provides a housing for necessary seal assemblies and an access point for a secondary lumen (for example, fluid lumen) in fluid communication with the central lumen of the sheath hub 20. The seal assembly 24, as described herein and as shown in FIGS. 5A and 5B, is included in the sheath hub 20. The seal assembly 24 includes a proximal seal 24a, an intermediate seal 24b, and a distal seal 24c. When assembled, the introducer 6 passes through the seal assembly and extends distal of the sheath 8. The proximal seal 24a, the intermediate seal 24b, and the distal seal 24c are each formed to prevent unwanted fluid from advancing in the proximal direction through the sheath hub 20 and proximal of the seal assembly 24. They are each openable and closable to provide pressure variation to affect the desired fluid flow from a physician or technician.

The distal end of the sheath hub 20 includes threads 21 for coupling to a threaded sheath hub cap 22. The sheath 8 is provided between the sheath hub 20 and the sheath hub cap 22 such that coupling the sheath hub cap 22 to the sheath hub 20 fixes the sheath 8 to the sheath hub 20. The sheath hub cap 22 is a cylindrical cap having a cap body having a proximal end and a distal end and defining a central lumen extending longitudinally between the proximal end and the distal end. The sheath hub cap 22 has a larger diameter at its proximal end than at its distal end.

The sheath hub 20 further has receiving slots 48 for coupling the sheath locking system 18, particularly the locking sleeve 28, to the sheath hub 20. The receiving slots 48 are openings which extend around a portion of the diameter of the sheath hub 20 and are sized and configured to accept the interference diameters 66 of the locking sleeve 28. Coupling between the receiving slots 48 and the interference diameters 66 axially and rotationally fixes the locking sleeve 28 and the sheath hub 20 relative to each other.

FIG. 2 illustrates the sheath 8 of FIG. 1 including a sheath locking system 18 which prevents axial and rotational translation of the introducer 6 with respect to the sheath 8. Example locking systems are disclosed in PCT/US2021/050006, entitled “Expandable Sheath Including Reverse Bayonet Locking Hub,” the disclosure of which is incorporated herein by reference. It is contemplated that the locking system disclosed herein can also be used to couple the sheath 8/sheath hub 20 with other delivery system components, catheters, dilators, etc. including the same mating features.

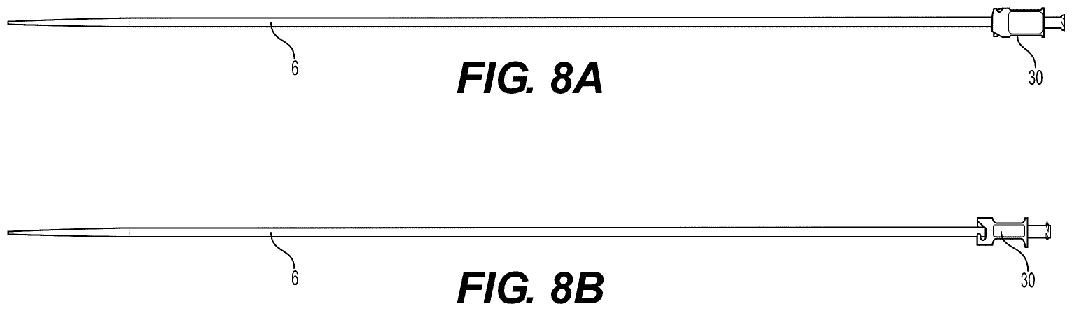

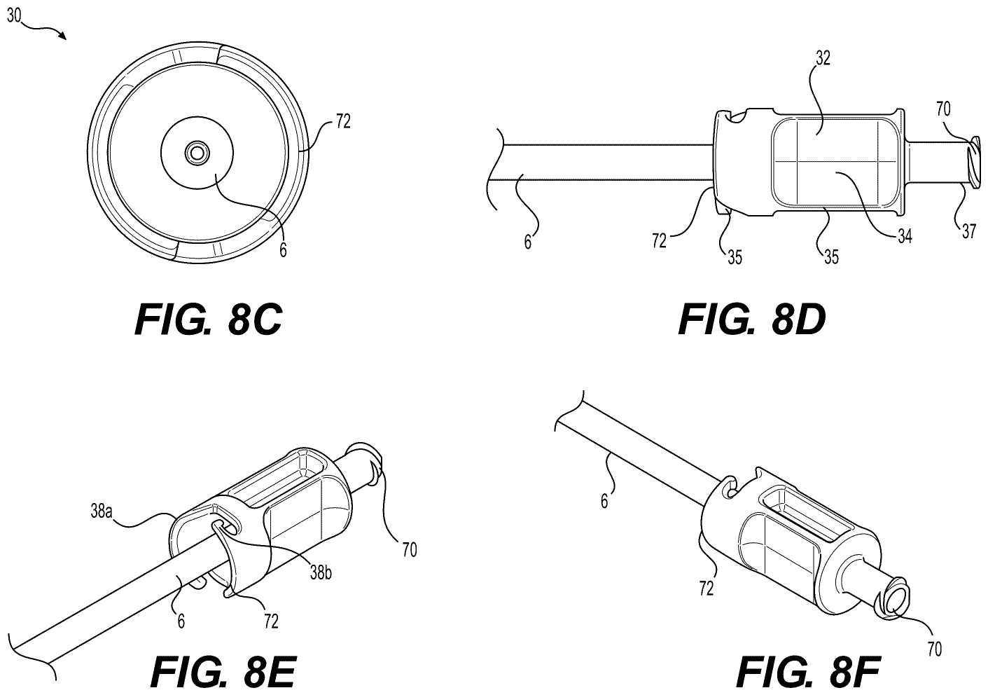

The sheath locking system 18 keeps the introducer 6 fixed with respect to the sheath 8 during insertion without requiring a physician or technician to hold the introducer 6 and the sheath 8 in place at the distal end. As illustrated in FIGS. 8A-8B, the sheath locking system 18 includes a locking sleeve 28 and an introducer locking hub 30 (including corresponding introducer 6). The locking sleeve 28 is coupled to the sheath 8 via the sheath hub 20. The locking sleeve 28 engages the introducer locking hub 30 and is moveable between a locked and unlocked position, thereby fixing the position of the introducer 6 and the sheath 8 and preventing movement therebetween, particularly during insertion into the patient. As will be described in more detail herein, the sheath locking system 18 keeps the introducer 6 from separating from the sheath 8 and prevents gaps from forming that can cause patient abrasions and unintended fluid flow between the introducer 6 and the sheath 8 during insertion.

FIGS. 2, 5A-5B and 6, and illustrate the sheath locking sleeve 28 coupled to the introducer locking hub 30 and the sheath hub 20. As will be described in more detail herein, the locking sleeve 28 includes a guide 31 that engages a locking channel 38 provided on the introducer locking hub 30. The guide 31 moves within the locking channel 38 between an unlocked position, where the sheath locking sleeve 28 is rotationally and axially movable with respect to the introducer locking hub 30, and a locked position (FIG. 2), where the locking sleeve 28 is axially fixed with respect to the introducer locking hub 30.

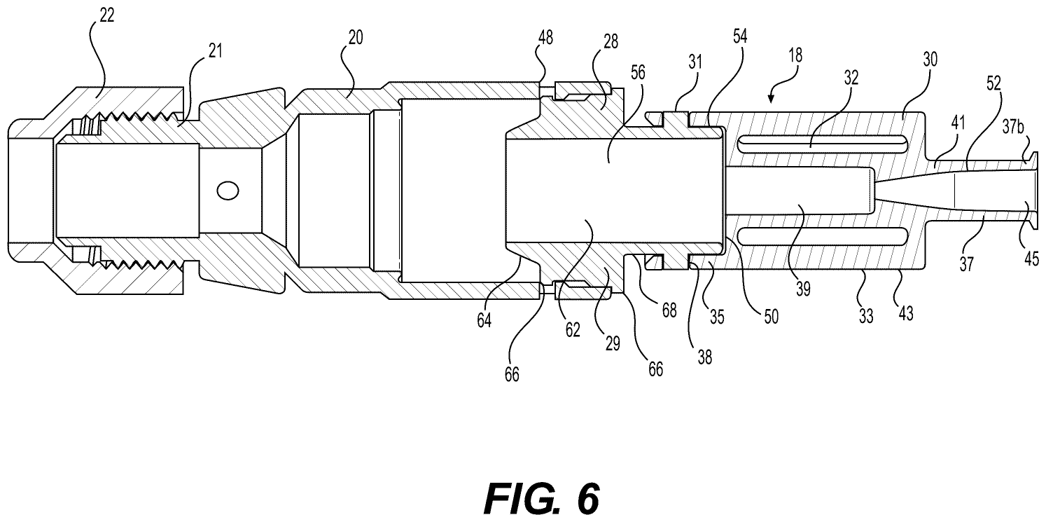

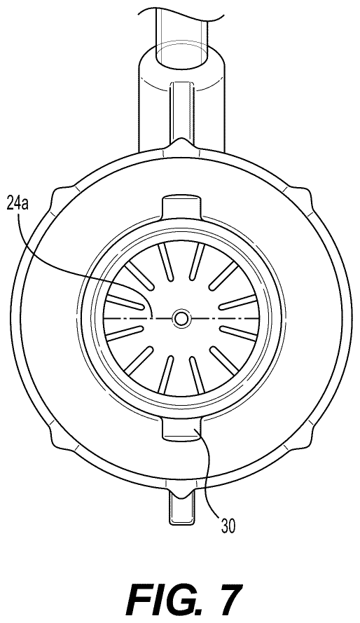

The locking sleeve 28 is illustrated, for example, in FIGS. 10A-10F. The locking sleeve 28 includes an elongated sleeve body 29 with a central lumen 56 extending longitudinally between the proximal end 58 and distal end 60 of the slot sleeve body 29. As provided in FIG. 6, the central lumen 56 defines a generally cylindrical inner surface 62 of the sheath locking sleeve 28. The central lumen 56 has a diameter of at least 0.3″. In some examples, the diameter ranges between 0.3″ and 0.6″. Preferably, the diameter is about 0.40″ The distal end 60 of the slot sleeve body 29 also has a frustoconical outer surface 64 that tapers about the distal end 60 to help with positioning the locking sleeve 28 within the sheath hub 20 and abutting the seal assembly 24 (FIGS. 5B and 5B). The locking sleeve 28 also has a plurality of interference diameters 66 that extend radially from the outer surface of the slot sleeve body 29 around (all or a portion of) the circumference of the locking sleeve 28. As illustrated in FIGS. 5A and 6, the distal interference diameters 66 are sized and configured to engage corresponding recesses and/or receiving slots 48 provided in the sheath hub 20 for securing the locking sleeve 28 to the sheath hub 20, and the distal interference diameter 66 seat against the proximal end of the sheath hub 20.

The locking sleeve 28 includes a guide 31 projecting from the outer surface 68 of the locking sleeve 28. The guide 31 engages a corresponding shaped locking channel 38 in the introducer locking hub 30. The guide 31 extends radially from the outer surface 68 and at least partially around the circumference of the outer surface 68. As provided in FIG. 6, the top surface of the guide 31 does not extend beyond the outer surface of the introducer locking hub 30 when the sheath locking sleeve 28 and the introducer locking hub 30 are coupled. For example, the height of the guide 31 corresponds to the wall thickness of the introducer locking hub 30 proximate the guide when the sheath locking sleeve 28 and the introducer locking hub 30 are coupled. In another example, the top surface of the guide 31 is recessed with respect to the outer surface of the introducer locking hub 30. That is, the height of the guide 31 is less than the wall thickness of the introducer locking hub 30. In other examples, the height of the guide 31 is greater than a wall thickness of the introducer locking hub 30 such that the top surface of the guide 31 extends beyond the outer surface of the introducer locking hub 30 when the sheath locking sleeve 28 and the introducer locking hub 30 are coupled. In some examples, the height/axial length of the guide 31 is between about 0.050″ and about 0.10.″ In some examples that height/axial length of the guide 31 is about 0.075″.

As illustrated in FIGS. 10D-10F, the guide 31 is a cylindrically shaped projection. However, it is contemplated that the guide 31 may have any other regular or irregular shape that would facilitate movement of the guide 31 within the locking channel 38 of the introducer locking hub 30. For example, the guide 31 may have an elongated hexagon shape. The guide 31 can have a diameter/width ranging from about 0.05″ to about 0.20″. Preferably the guide 31 has a diameter/width of about 0.100″.

In general, the locking sleeve 28 can be formed from polycarbonate, but in other aspects, the locking sleeve 28 can be formed from rigid plastic, or any other material suitable for providing a strong locking connector for an introducer 6 (metal, composite, etc.).

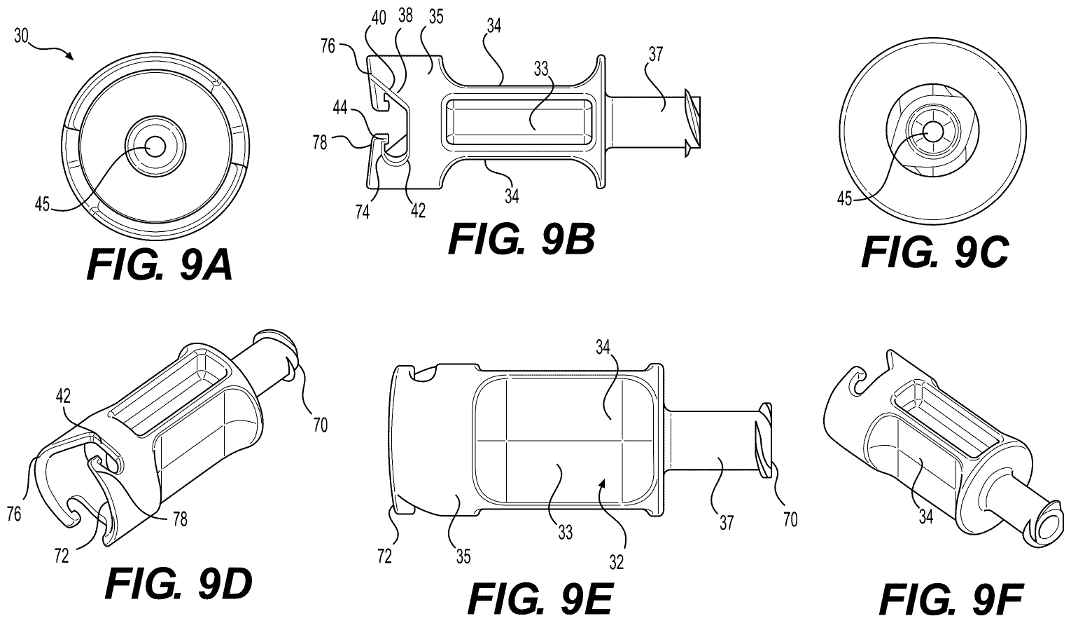

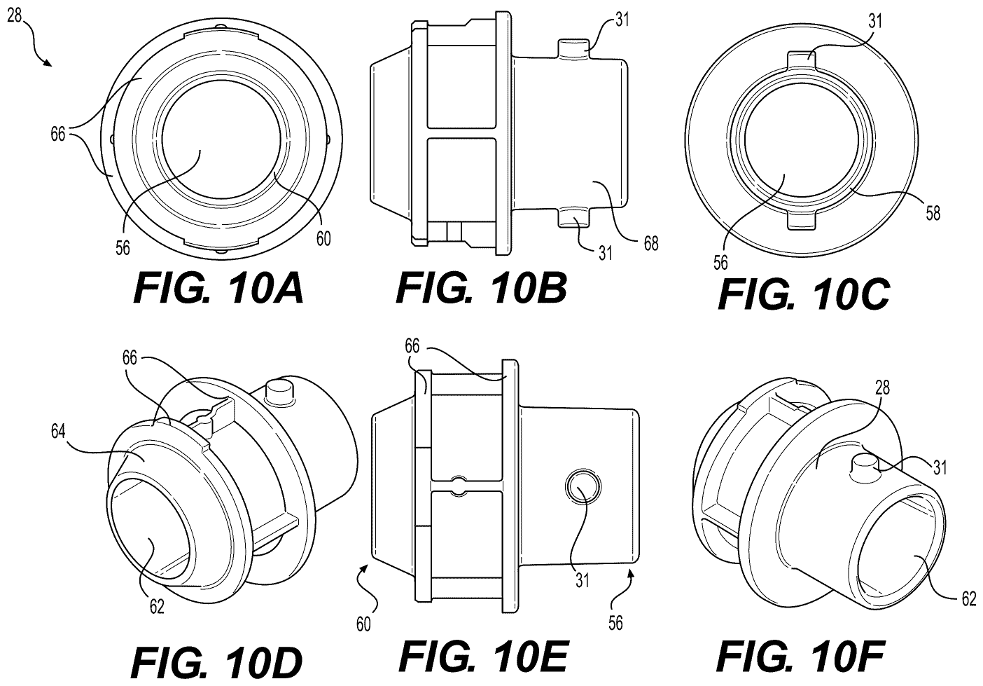

FIGS. 2-6 illustrate the introducer locking hub 30 coupled to the locking sleeve 28. FIGS. 8A-8F show the introducer locking hub 30 coupled to the introducer 6. FIGS. 9A-9F provide multiple views of the introducer locking hub 30. As described herein, the introducer 6 is fixedly coupled to the introducer locking hub 30. The introducer locking hub 30 couples with the locking sleeve 28 to fix the position the introducer 6 (axially and rotationally) with respect to the locking sleeve 28/sheath 8. Each of the introducer 6 and introducer locking hub 30 are described in more detail herein.

FIGS. 8A-8F illustrate the introducer locking hub 30 with the introducer 6 coupled thereto. Example introducer sheaths are described, for example in U.S. Pat. Nos. 8,690,936 and 8,790,387, the disclosures of which are incorporated herein by reference. As provided in the cross-section views of FIGS. 5A and 5B, the introducer 6 is coupled to the introducer locking hub 30 and extends beyond the distal end of the introducer locking hub 30 body and into the sheath 8. When coupled to the sheath hub 20, the introducer 6 extends through the central lumen 56 of the sheath locking sleeve 28, the sheath hub 20 and the central lumen of the sheath 8. As will be described herein, the sheath 8 generally comprises a radially expandable tubular structure. Passage of the introducer 6 through the sheath 8 and into a patient's vasculature causes the blood vessel to radially expand to about the diameter of the sheath 8. That is, the diameter of the central lumen of the sheath 8 is generally abuts the outer diameter of the introducer 6 such that the introducer 6 provides a mechanism to expand a patient's vessel to accept the sheath.

As provided in FIGS. 8A-8F, the introducer 6 is formed as an elongate body with a central lumen extending therethrough. As shown in FIGS. 5A and 5B, the central lumen of the introducer is aligned with the central lumens of the introducer locking hub 30, the sheath hub 20 and the sheath 8. The introducer 6 is received within a recessed opening 39 provided on an interior surface of the introducer locking hub 30, the recessed opening 39 axially aligned with the central lumen 45 of the introducer locking hub 30. The introducer 6 is coupled to the introducer locking hub 30 at the recessed opening 39. In an example system, the introducer 6 has a diameter corresponding to, or less than, the diameter of the recessed opening 39. In some examples, the introducer 6 is fixedly coupled to the introducer locking hub 30 at the recessed opening 39. For example, the introducer 6 is coupled to the recessed opening 39 of the introducer locking hub 30 by at least one of a press fit, an interference fit, a snap fit, a mechanical fastener, a chemical fastener (for example, an adhesive), a weld, a thermal process, and/or any other suitable coupling process known in the art.

As described herein, the introducer 6 has a central lumen that aligns with the central lumen 45 of the introducer locking hub 30. This joined lumen allows for the passage of surgical equipment and/or medical devices to the treatment site (for example, a guide wire). In an example system, and as provided in FIGS. 5A and 5B, the central lumen of the introducer 6 has a diameter corresponding to at least a portion of the diameter of the central lumen 45 of the introducer locking hub 30. In general, the corresponding diameter portion is adjacent the distal end of the central lumen 45. In other examples, the diameter of the central lumen 45 at the distal end of the introducer locking hub 30 is slightly larger than the diameter of the central lumen passing through the introducer 6. The central lumen 45 can also define a decreasing tapered portion 41 between the proximal end and the distal end of the introducer locking hub 30 (see FIG. 6). The corresponding diameter portion and decreasing tapered portion 41 allows for smooth transition and delivery of surgical equipment and/or medical device through the introducer locking hub 30 and into the central lumen of the introducer 6.

As illustrated in FIGS. 9A-9F, the introducer locking hub 30 includes a hub body 32 having a proximal end 70 and a distal end 72 and defining a central lumen 45 extending therethrough. The hub body 32 has a first (middle) portion 33, a second (distal) portion 35 which extends distally from the first portion 33 and a third (proximal) portion 37 which extends proximally from the first portion 33. The first portion 33 includes the cylindrically-shaped recessed opening 39 for receiving and retaining the introducer 6 and an outer surface 43. In some examples, the recessed opening 39 has a diameter ranging between 0.15″ and about 0.25″. In some examples, the recessed opening 39 has a diameter ranging between 0.17″ and about 0.20″. In some examples, the recessed opening has a diameter of about 0.194″.

The third (proximal) portion 37 of the introducer locking hub 30 includes the decreasing tapered portion 41 of the central lumen 45. The decreasing tapered portion 41 defining a frustoconical shape with decreasing taper/diameter from the proximal to the distal end of the sheath. It is contemplated that the tapered portion 41 has a minimum diameter of about 0.007″ and a maximum diameter of about 0.194″.

As illustrated in FIGS. 5A and B, when coupled, the central lumen 56 of the locking sleeve 28 is aligned with the central lumen 45 of the introducer locking hub 30. In some examples, the central lumen 56 of the locking sleeve 28 is coaxial with the central lumen 45 of the introducer locking hub 30. When coupled, the proximal end of the locking sleeve 28 is received within the central lumen 45 of the introducer locking hub 30. The proximal end surface of the locking sleeve 28 is adjacent a shoulder 50 provided on an inner surface of the central lumen 45 of the introducer locking hub 30. As illustrated in FIGS. 5A and 5B, the central lumen 45 of the introducer locking hub 30 includes a first portion 52 having a first diameter adjacent the proximal end of the introducer locking hub 30, and a second portion 54 having a second, larger, diameter adjacent the distal end of the introducer locking hub 30. The recessed opening 39 can be considered either a component of the first portion 52 of the central lumen 45, or a separate component of the central lumen 45 located between the first (proximal) portion 52 and the second (distal) portion 54. When the locking sleeve 28 and introducer locking hub 30 are coupled, at least a portion of the slot sleeve body 29 of the sheath locking sleeve 28 is received within the second portion 54 (larger portion) of the central lumen 45 of the introducer locking hub 30. The central lumen 56 of the sheath locking sleeve 28 is aligned with the central lumen 45 of the introducer locking hub 30 such that they are co-axial and form a smooth inner surface along the combined central lumens of the introducer locking hub 30 and the sheath locking sleeve 28.

As described generally herein, the locking sleeve 28 couples to the introducer locking hub 30 via engagement between the guide 31 on the locking sleeve 28 and the locking channel 38 provided in the introducer locking hub 30. As provided in FIGS. 9A-9F, the introducer locking hub 30 includes two locking channels 38. However, it is contemplated that the introducer locking hub 30 can include one locking channel 38 or more than two locking channels 38. The locking channel 38 can be is formed a recess or groove in a surface of the introducer locking hub 30, as a slotted opening, a clip, or as any other feature capable of receiving and securing the guide 31 projecting from the outer surface of the locking sleeve 28 with the introducer locking hub 30. Illustrated in FIG. 9B, the locking channels 38 provide an interface to secure the sheath locking sleeve 28 to the introducer locking hub 30 and ensure a fixed axial position between the introducer 6 and the sheath 8.

The locking channel 38 is formed on the distal end of the introducer locking hub 30. The locking channel 38 includes an opening on the distal end surface that leads to an angled guide portion 40 that transitions to a locking portion 42. The guide portion 40 is configured to direct the guide 31 of the locking sleeve 28 in an axial and circumferential direction along the side wall of the guide portion 40 towards the locking portion 42 upon rotation of the introducer locking hub 30 and/or the sheath locking sleeve 28. The locking portion 42 is configured to securely engage the guide 31, fixing the axial position of the introducer locking hub 30 with respect to the sheath locking sleeve 28. As illustrated in FIG. 9B, the guide portion 40 of the locking channel 38 extends from the distal end of the introducer locking hub 30 axially towards the proximal end of the introducer locking hub 30 and circumferentially around the introducer locking hub 30. For example, the guide portion 40 of the locking channel 38 can be described as extending helically around/along a length of the introducer locking hub 30 or on an angle from the distal end of the introducer locking hub 30.

As illustrated in FIGS. 9B and 9D, the locking portion 42 of the locking channel 38 extends at an angle from the end of the guide portion 40. As provided in FIG. 9B, the angle between the centerline of the guide portion 40 and the centerline of the locking portion 42 is greater than 90-degrees. In another example, the angle between the centerline of the guide portion 40 and the centerline of the locking portion 42 is about 120-degrees. In an example system, the locking portion 42 extends around a portion of the circumference of the introducer locking hub 30. The locking portion 42 can extend parallel to the distal end of the introducer locking hub 30. In an example system, the length of the guide portion 40 (measured along its centerline) is greater than the length of the locking portion 42 (measured along its centerline). In another example, the length of the guide portion 40 equals or is less than a length of the locking portion 42.

The locking portion 42 can include a catch 44 for securing the guide 31 within the locking portion 42 of the locking channel 38 and forming a partial barrier for the guide 31 within the locking portion 42. As illustrated in FIG. 9B, the catch 44 includes a projection that extends from a side wall 74 of the locking portion 42 and releasably secures the guide 31 within the locking channel 38. The catch 44 extends from the side wall 42a of the locking portion 42 in a proximal direction towards the center line of the locking portion 42 and has a height sufficient to retain the guide 31 between the catch 44 and the end of the locking portion 42.

The distal end surface 72 of the introducer locking hub 30 can include features for biasing the guide 31 towards the locking channel 38. For example, the distal end of the introducer locking hub 30 can include a tapered surface angled toward an opening of the locking channel 38. As illustrated in FIG. 9B, the distal end 72 of the introducer locking hub 30 includes a first tapered surface 76 (angled towards a leading edge of the opening of the locking channel 38 and a second tapered surface 78 angled towards the trailing edge of the opening of the locking channel 38.

In use, engagement between the guide 31 and the guide portion 40 of the locking channel 38 is configured to bias the locking sleeve 28 in a proximal axial direction toward the proximal end 70 of the introducer locking hub 30 (towards a locked position) when the sheath locking sleeve 28 is rotated in a first axial direction. In this direction the guide 31 advances toward the locking portion 42 of the locking channel 38 into the locked position. Alternatively, engagement between the guide 31 and the locking portion 42 of the locking channel 38 is configured to bias the locking sleeve 28 in a distal axial direction toward the distal end of the introducer locking hub 30 (towards an unlocked position) when the sheath locking sleeve 28 is rotated in a second (opposite) axial direction. In the second direction, the guide 31 advances away from the locking portion 42 of the locking channel 38, to the unlocked position. When the guide 31 is in the locked position and retained with by locking portion 42 by catch 44, rotation in the second direction causes the guide 31 to bias against the catch 44 overcoming the oppositional forces of the catch 44, and moving the guide 31 from the locked to the unlocked position.

As illustrated in FIGS. 8A-9F, the outer surface of the introducer locking hub body 32 includes gripping features and/or surfaces for a physician or technician to use when manipulating the introducer locking hub 30. As provided in FIG. 9B, the introducer locking hub body 32 can include a two recessed gripping surfaces 34 on opposite sides of the longitudinal axis of the introducer locking hub 30. When the introducer locking hub 30 is viewed from the side, the gripping surfaces 34 define a dog-bone/barbell shape to the hub body 32, i.e., a shape having a smaller diameter/width center portion and larger diameter/width end portions. In an example system, the gripping surfaces 34 are provided along at least 40% of the length of the introducer locking hub body 32. In another example, the gripping surfaces 34 are provided along at least 50% of the length of the introducer locking hub body 32.

In general, the introducer locking hub 30 can be formed from polycarbonate, but in other aspects the introducer locking hub 30 can be formed from rigid plastic, or any other material suitable for providing a locking mechanism for an introducer 6 (metal, composite, etc.).

As described herein, the introducer device/sheath assembly includes an expandable sheath 8 extending distally from the sheath hub 20. The expandable sheath 8 has a central lumen to guide passage of the delivery apparatus 10 for the medical device/prosthetic heart valve. In an alternative aspect, the introducer device/sheath assembly need not include the sheath hub 20. For example, the sheath 8 can be an integral part of a component of the sheath assembly, such as the guide catheter. As described herein, the sheath 8 can have a natural, unexpanded outer diameter that will expand locally upon passage of the medical device.

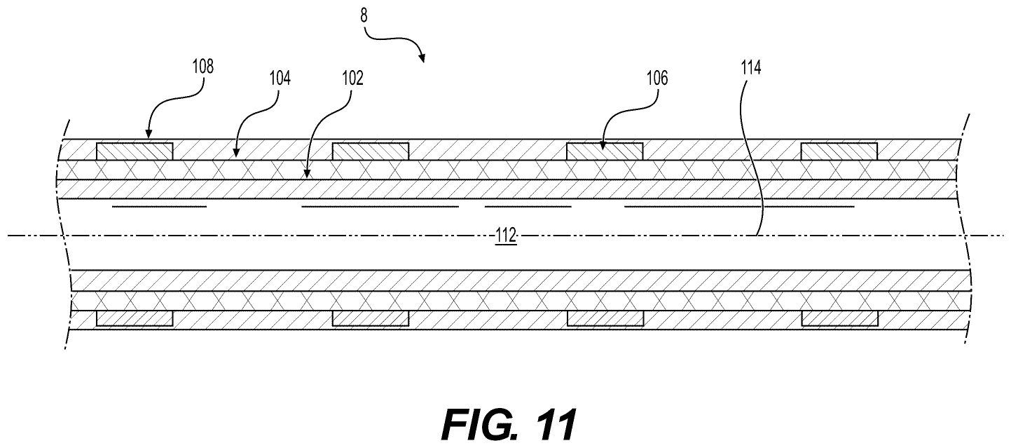

In certain aspects, the expandable sheath 8 can comprise a plurality of coaxial layers extending along at least a portion of the length of the sheath 8. The structure of the coaxial layers is described in more detail herein with respect to FIGS. 11-23. Example expandable sheaths including coaxial layers are described, for example, in U.S. patent application Ser. No. 16/378,417, entitled “Expandable Sheath,” and U.S. patent application Ser. No. 17/716,882, entitled “Expandable Sheath,” the disclosures of which are herein incorporated by reference.

Various aspects of the coaxial layered structure of the sheath 8 are described herein. For example, in reference to the example sheath 8 illustrated in FIGS. 11-14, the expandable sheath 8 can include a number of layers including an inner layer 102 (also referred to as an inner layer), a second layer 104 disposed around and radially outward of the inner layer 102, a third layer 106 disposed around and radially outward of the second layer 104, and a fourth outer layer 108 (also referred to as an outer layer) disposed around and radially outward of the third layer 106. In the illustrated configuration, the inner layer 102 can define the lumen 112 of the sheath extending along a central axis 114 through which the delivery apparatus travels into the patient's vessel in order to deliver, remove, repair, and/or replace a prosthetic device, moving in a direction along the longitudinal axis of the sheath 8.

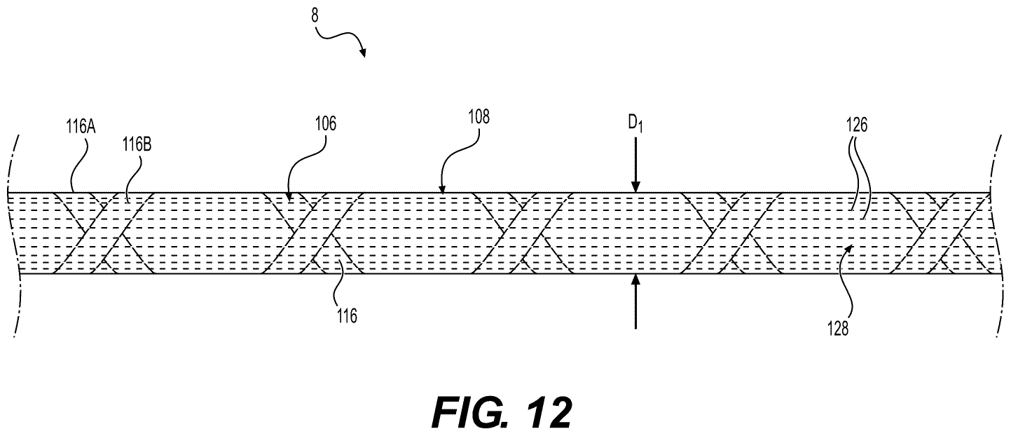

Referring to FIG. 12, when the sheath 8 is in an unexpanded state, various layers of the sheath, for example, the inner layer 102 and/or the outer layer 108, can form longitudinally-extending folds or creases such that the surface of the sheath comprises a plurality of ridges 126 (also referred to herein as “folds”). The ridges 126 can be circumferentially spaced apart from each other by longitudinally-extending valleys 128. When the sheath expands beyond its natural diameter D₁, the ridges 126 and the valleys 128 can level out or be taken up as the surface radially expands and the circumference increases, as further described herein. When the sheath 8 collapses back to its natural diameter, the ridges 126 and valleys 128 can reform.

In certain aspects, the inner layer 102 and/or the outer layer 108 can comprise a relatively thin layer of polymeric material. For example, in some aspects the thickness of the inner layer 102 can be from 0.01 mm to 0.5 mm, 0.02 mm to 0.4 mm, or 0.03 mm to 0.25 mm. In certain aspects, the thickness of the outer layer 108 can be from 0.01 mm to 0.5 mm, 0.02 mm to 0.4 mm, or 0.03 mm to 0.25 mm.

In certain examples, the inner layer 102 and/or the outer layer 108 can comprise a lubricious, low-friction, and/or relatively non-elastic material. In particular aspects, the inner layer 102 and/or the outer layer 108 can comprise a polymeric material having a modulus of elasticity of 400 MPa or greater. Exemplary materials can include ultra-high-molecular-weight polyethylene (UHMWPE) (for example, Dyneema®), high-molecular-weight polyethylene (HMWPE), or polyether ether ketone (PEEK). With regard to the inner layer 102 in particular, such low coefficient of friction materials can facilitate passage of the prosthetic device through the lumen 112. Other suitable materials for the inner layer 102 and outer layer 108 can include polyimide, polytetrafluoroethylene (PTFE), expanded polytetrafluoroethylene (ePTFE), ethylene tetrafluoroethylene (ETFE), nylon, polyethylene, polyamide, polyether block amide (for example, Pebax), and/or combinations of any of the materials described herein. Some aspects the sheath 8 can include a lubricious liner on the inner surface of the inner layer 102. Examples of suitable lubricious liners include materials that can further reduce the coefficient of friction of the inner layer 102, such as PTFE, polyethylene, polyvinylidine fluoride, and combinations thereof. Suitable materials for a lubricious liner also include other materials desirably having a coefficient of friction of 0.1 or less.

Additionally, some aspects of the sheath 8 can include an exterior hydrophilic coating on the outer surface of the outer layer 108. Such a hydrophilic coating can facilitate insertion of the sheath 8 into a patient's vessel, reducing potential damage. Examples of suitable hydrophilic coatings include the Harmony™ Advanced Lubricity Coatings and other Advanced Hydrophilic Coatings available from SurModics, Inc., Eden Prairie, MN. DSM medical coatings (available from Koninklijke DSM N. V, Heerlen, the Netherlands), as well as other hydrophilic coatings (for example, PTFE, polyethylene, polyvinylidine fluoride), are also suitable for use with the sheath 8. Such hydrophilic coatings may also be included on the inner surface of the inner layer 102 to reduce friction between the sheath and the delivery system, thereby facilitating use and improving safety. In some aspects, a hydrophobic coating, such as Perylene, may be used on the outer surface of the outer layer 108 or the inner surface of the inner layer 102 in order to reduce friction.

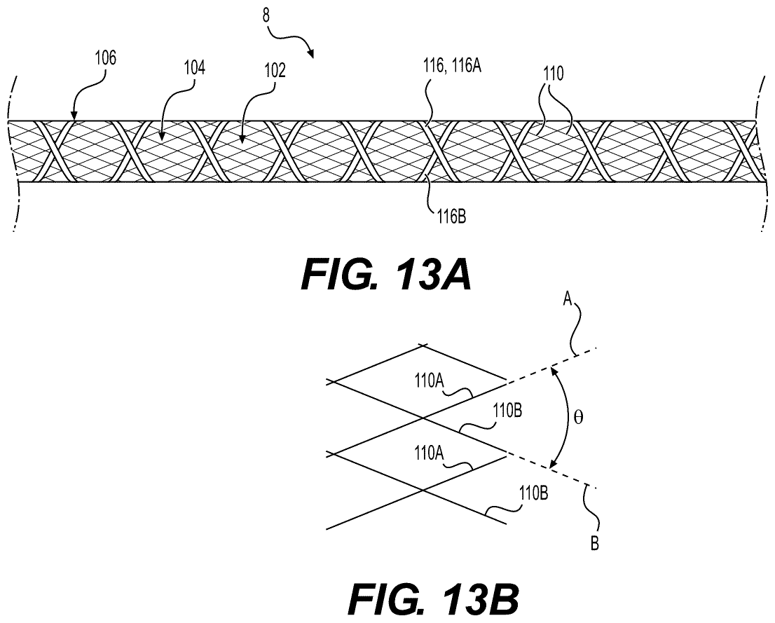

In certain aspects, the second layer 104 can be a braided layer. FIGS. 13A and 13B illustrate the sheath 8 with the outer layer 108 removed to expose the elastic third layer 106. With reference to FIGS. 13A and 13B, the braided second layer 104 can comprise a plurality of members or filaments 110 (for example, metallic or synthetic wires or fibers) braided together. The braided second layer 104 can have any desired number of filaments 110, which can be oriented and braided together along any suitable number of axes. For example, with reference to FIG. 13B, the filaments 110 can include a first set of filaments 110A oriented parallel to a first axis A, and a second set of filaments 110B oriented parallel to a second axis B. The filaments 110A and 110B can be braided together in a biaxial braid such that filaments 110A oriented along axis A form an angle θ with the filaments 110B oriented along axis B. In certain aspects, the angle θ can be from 5° to 70°, 10° to 60°, 10° to 50°, or 10° to 45°. In the illustrated example, the angle θ is 45°. In other aspects, the filaments 110 can also be oriented along three axes and braided in a triaxial braid, or oriented along any number of axes and braided in any suitable braid pattern. The braided second layer 104 can extend along substantially the entire length L of the sheath 8, or alternatively, can extend only along a portion of the length of the sheath. In particular aspects, the filaments 110 can be wires made from metal (for example, Nitinol, stainless steel, etc.), or any of various polymers or polymer composite materials, such as carbon fiber. In certain aspects, the filaments 110 can be round, and can have a diameter of from 0.01 mm to 0.5 mm, 0.03 mm to 0.4 mm, or 0.05 mm to 0.25 mm. In other aspects, the filaments 110 can have a flat cross-section with dimensions of 0.01 mm×0.01 mm to 0.5 mm×0.5 mm, or 0.05 mm×0.05 mm to 0.25 mm×0.25 mm. In one aspect, filaments 110 having a flat cross-section can have dimensions of 0.1 mm×0.2 mm. However, other geometries and sizes are also suitable for certain aspects. If braided wire is used, the braid density can be varied. Some aspects have a braid density of from ten picks per inch to eighty picks per inch, and can include eight wires, sixteen wires, or up to fifty-two wires in various braid patterns. In other aspects, the second layer 104 can be laser cut from a tube, or laser-cut, stamped, punched, etc., from sheet stock and rolled into a tubular configuration. The second layer 104 can also be woven or knitted, as desired.

The third layer 106 can be a resilient, elastic layer (also referred to as an clastic material layer). In certain aspects, the elastic third layer 106 can be configured to apply radially inward force to the underlying layers 102 and 104 in a radial direction (for example, toward the central axis 114 of the sheath) when the sheath expands beyond its natural diameter by passage of the delivery apparatus through the sheath. Stated differently, the elastic third layer 106 can be configured to apply encircling/radially inward pressure to the layers of the sheath beneath the elastic third layer 106 to counteract expansion of the sheath. The radially inwardly directed force is sufficient to cause the sheath to collapse radially back to its unexpanded state after the delivery apparatus is passed through the sheath.

In the illustrated example, the elastic third layer 106 can comprise one or more members configured as strands, ribbons, or bands 116 helically wrapped around the braided second layer 104. For example, in the illustrated aspect the elastic third layer 106 comprises two elastic bands 116A and 116B wrapped around the braided second layer 104 with opposite helicity, although the elastic layer may comprise any number of bands depending upon the desired characteristics. The elastic bands 116A and 116B can be made from, for example, any of a variety of natural or synthetic elastomers, including silicone rubber, natural rubber, any of various thermoplastic elastomers, polyurethanes such as polyurethane siloxane copolymers, urethane, plasticized polyvinyl chloride (PVC), styrenic block copolymers, polyolefin elastomers, etc. In some aspects, the elastic layer can comprise an elastomeric material having a modulus of elasticity of 200 MPa or less. In some aspects, the elastic third layer 106 can comprise a material exhibiting an elongation to break of 200% or greater, or an elongation to break of 400% or greater. The elastic third layer 106 can also take other forms, such as a tubular layer comprising an elastomeric material, a mesh, a shrinkable polymer layer such as a heat-shrink tubing layer, etc. In lieu of, or in addition to, the elastic third layer 106, the sheath 8 may also include an elastomeric or heat-shrink tubing layer around the outer layer 108. Examples of such elastomeric layers are disclosed in U.S. Publication No. 2014/0379067, U.S. Publication No. 2016/0296730, and U.S. Publication No. 2018/0008407, which are incorporated herein by reference. In other aspects, the elastic third layer 106 can also be radially outward of the polymeric outer layer 108.

In certain aspects, one or both of the inner layer 102 and/or the outer layer 108 can be configured to resist axial elongation of the sheath 8 when the sheath expands. More particularly, one or both of the inner layer 102 and/or the outer layer 108 can resist stretching against longitudinal forces caused by friction between a prosthetic device and the inner surface of the sheath 8 such that the length L remains substantially constant as the sheath expands and contracts. As used herein with reference to the length L of the sheath, the term “substantially constant” means that the length L of the sheath increases by not more than 1%, by not more than 5%, by not more than 10%, by not more than 15%, or by not more than 20%. Meanwhile, with reference to FIG. 13B, the filaments 110A and 110B of the braided second layer 104 can be allowed to move angularly relative to each other such that the angle θ changes as the sheath expands and contracts. This, in combination with the longitudinal folds/ridges 126 in the inner layer 102 and the outer layer 108, can allow the lumen 112 of the sheath to expand as a prosthetic device is advanced through it.

For example, in some aspects the inner layer 102 and the outer layer 108 can be heat-bonded during the manufacturing process such that the braided second layer 104 and the elastic third layer 106 are encapsulated between the inner layer 102 and the outer layer 108. More specifically, in certain aspects the inner layer 102 and the outer layer 108 can be adhered to each other through the spaces between the filaments 110 of the braided second layer 104 and/or the spaces between the elastic bands 116. The inner layer 102 and the outer layer 108 can also be bonded or adhered together at the proximal and/or distal ends of the sheath. In certain aspects, the inner layer 102 and outer layer 108 are not adhered to the filaments 110. This can allow the filaments 110 to move angularly relative to each other, and relative to the inner layer 102 and outer layer 108, allowing the diameter of the braided second layer 104, and thereby the diameter of the sheath, to increase or decrease. As the angle θ between the filaments 110A and 110B changes, the length of the braided second layer 104 can also change. For example, as the angle θ increases, the braided second layer 104 can foreshorten, and as the angle θ decreases, the braided second layer 104 can lengthen to the extent permitted by the areas where the inner layer 102 and outer layer 108 are bonded. However, because the braided second layer 104 is not adhered to the inner layer 102 and outer layer 108, the change in length of the braided layer that accompanies a change in the angle θ between the filaments 110A and 110B does not result in a significant change in the length L of the sheath.

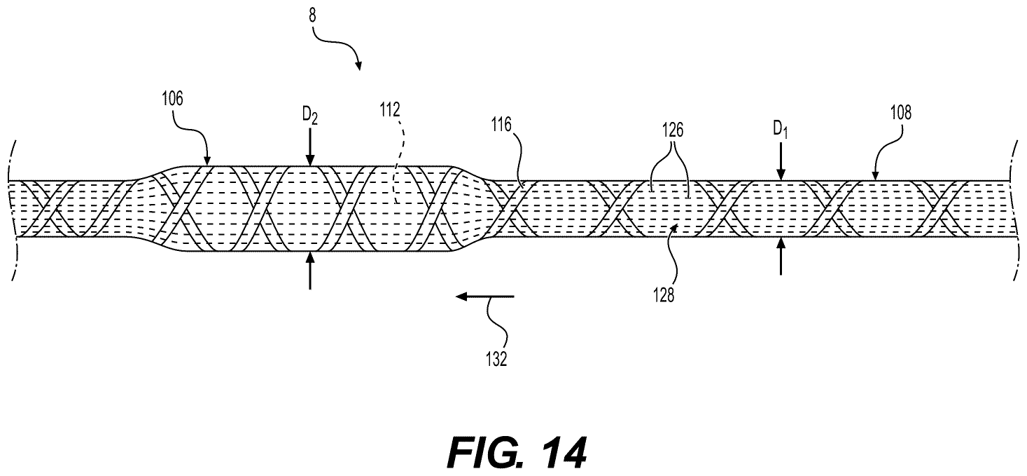

FIG. 14 illustrates radial expansion of the sheath 8 as a prosthetic device (for example, implant 12) is passed through the sheath 8 in the direction of arrow 132 (for example, distally). As the prosthetic device (implant 12) is advanced through the sheath 8, the sheath can resiliently expand to a second diameter D₂ that corresponds to a size or diameter of the prosthetic device. As the prosthetic device (implant 12) is advanced through the sheath 8, the prosthetic device can apply longitudinal force to the sheath in the direction of motion by virtue of the frictional contact between the prosthetic device and the inner surface of the sheath. However, as noted herein, the inner layer 102 and/or the outer layer 108 can resist axial elongation such that the length L of the sheath remains constant, or substantially constant. This can reduce or prevent the braided second layer 104 from lengthening, and thereby constricting the lumen 112.

Meanwhile, the angle θ between the filaments 110A and 110B can increase as the sheath expands to the second diameter D₂ to accommodate the prosthetic valve. This can cause the braided second layer 104 to foreshorten. However, because the filaments 110 are not engaged or adhered to the inner layer 102 or the outer layer 108, the shortening of the braided second layer 104 attendant to an increase in the angle θ does not affect the overall length L of the sheath. Moreover, because of the longitudinally-extending folds/ridges 126 formed in the inner layer 102 and outer layer 108, the inner layer 102 and outer layer 108 can expand to the second diameter D₂ without rupturing, in spite of being relatively thin and relatively non-elastic. In this manner, the sheath 8 can resiliently expand from its natural diameter D₁ to a second diameter D₂ that is larger than the diameter D₁ as a prosthetic device is advanced through the sheath, without lengthening, and without constricting. Thus, the force required to push the prosthetic implant through the sheath is significantly reduced.

Additionally, because of the radial force applied by the elastic third layer 106, the radial expansion of the sheath 8 can be localized to the specific portion of the sheath occupied by the prosthetic device. For example, with reference to FIG. 14, as the prosthetic device (implant 12) moves distally through the sheath 8, the portion of the sheath immediately proximal to the prosthetic device (for example, implant 12) can radially collapse back to the initial diameter D₁ under the influence of the elastic third layer 106. The inner layer 102 and the outer layer 108 can also buckle as the circumference of the sheath is reduced, causing the ridges 126 and the valleys 128 to reform. This can reduce the size of the sheath required to introduce a prosthetic device of a given size. Additionally, the temporary, localized nature of the expansion can reduce trauma to the blood vessel into which the sheath is inserted, along with the surrounding tissue, because only the portion of the sheath occupied by the prosthetic device expands beyond the sheath's natural diameter and the sheath collapses back to the initial diameter once the device has passed. This limits the amount of tissue that must be stretched in order to introduce the prosthetic device, and the amount of time for which a given portion of the vessel must be dilated.

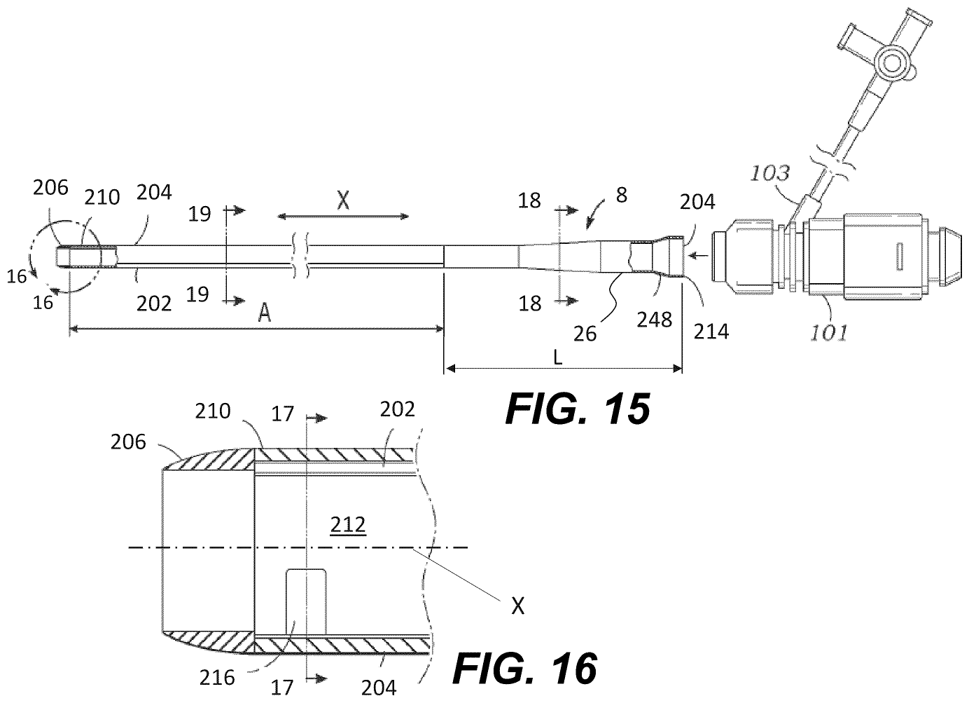

In another example layered sheath 8 structure, FIGS. 15-23 illustrate various features of the coaxial layered structure of the expandable sheath 8 of FIG. 1 according to another aspect. Similar reference numbers are used to describe like elements. It is to be understood that the variations (for example, materials and alternate configurations) described herein with reference to FIGS. 11-14 can also apply to the example shown in FIGS. 15-23. Furthermore, the variations described herein with reference to FIGS. 15-23 can also be applied to the sheath described in FIGS. 11-14.

Similar to various aspects of the sheath 8 described herein in reference to FIGS. 11-14, the sheath 8 of FIGS. 15-23 includes a plurality of layers. For example, the sheath 8 illustrated in FIGS. 15-23, also includes an inner layer 202 and an outer layer 204 disposed around the inner layer 202. The inner layer 202 can define a lumen 212 through which the delivery apparatus travels into the patient's vessel in order to deliver, remove, repair, and/or replace a prosthetic device, moving in a direction along the longitudinal axis X. Similar to the sheath illustrated in FIGS. 11-14, as the prosthetic device passes through the sheath 8, the sheath 8 locally expands from a first, resting/unexpanded diameter to a second, expanded diameter to accommodate the prosthetic device. After the prosthetic device passes through a particular location of the sheath 8, each successive expanded portion or segment of the sheath 8 at least partially returns to the smaller, resting/unexpanded diameter. In this manner, the sheath 8 can be considered self-expanding, in that it does not require use of a balloon, dilator, and/or obturator to expand.

Similar to the examples herein, the inner and outer layers 202, 204 can comprise any suitable materials. Suitable materials for the inner layer 202 include polytetrafluoroethylene (PTFE), ethylene tetrafluoroethylene (ETFE), nylon, polyethylene, polyether block amide (for example, Pebax), and/or combinations thereof. In one specific implementation the inner layer 202 can comprise a lubricious, low-friction, or hydrophilic material, such as PTFE. Such low coefficient of friction materials can facilitate passage of the prosthetic device through the lumen defined by the inner layer 202. In some examples, the inner layer 202 can have a coefficient of friction of less than about 0.1. Some examples of the sheath 8 can include a lubricious liner on the inner surface of the inner layer 202. Examples of suitable lubricious liners include materials that can further reduce the coefficient of friction of the inner layer 202, such as PTFE, polyethylene, polyvinylidene fluoride, and combinations thereof. Suitable materials for a lubricious liner also include other materials desirably having a coefficient of friction of about 0.1 or less.

Suitable materials for the outer layer 204 include nylon, polyethylene, Pebax, HDPE, polyurethanes (for example, Tecoflex), and other medical grade materials. In one implementation, the outer layer 204 can comprise high density polyethylene (HDPE) and Tecoflex (or other polyurethane material) extruded as a composite. In some implementations, the Tecoflex can act as an adhesive between the inner layer 202 and the outer layer 204 and may only be present along a portion of the inner surface of the outer layer 204. Other suitable materials for the inner and outer layers are also disclosed in U.S. Pat. Nos. 8,690,936 and 8,790,387, which are incorporated herein by reference.

Additionally, some examples of the sheath 8 include an exterior hydrophilic coating on the outer surface of the outer layer 204. Such a hydrophilic coating can facilitate insertion of the sheath 100 into a patient's vessel. Examples of suitable hydrophilic coatings include the Harmony™ Advanced Lubricity Coatings and other Advanced Hydrophilic Coatings available from SurModics, Inc., Eden Prairie, MN. DSM medical coatings (available from Koninklijke DSM N.V, Heerlen, the Netherlands), as well as other hydrophilic coatings (for example, PTFE, polyethylene, polyvinylidene fluoride), are also suitable for use with the sheath 100.

FIG. 16 provides a partial cross-section of the distal end of the sheath 8 along section line 16-16 identified in FIG. 15. As described herein, the sheath 8 can be inserted into a vessel (for example, the femoral or iliac arteries) by passing through the skin of patient, such that a soft tip portion 206 at the distal end 210 of the sheath 8 is inserted into the vessel. As best seen in FIG. 16, the soft tip portion 206 can comprise, in some examples, low density polyethylene (LDPE) and can be configured to minimize trauma or damage to the patient's vessels as the sheath is navigated through the vasculature. For example, the soft tip portion 206 can be slightly tapered to facilitate passage through the vessels. The soft tip portion 206 can be secured to the distal end 210 of the sheath 8, such as by thermally bonding the soft tip portion 206 to the inner and outer layers 202, 204 of the sheath 8. Such a soft tip portion 206 can be provided with a lower hardness than the other portions of the sheath 8. In some examples, the soft tip portion 206 can have a Shore hardness from about 25 D to about 40 D. The soft tip portion 206 is configured to be radially expandable to allow a prosthetic device to pass through the distal opening of the sheath 208. For example, the soft tip portion 206 can be formed with a weakened portion, such as an axially extending score line or perforated line that is configured to split and allow the soft tip portion 206 to expand radially when the prosthetic device passes therethrough.

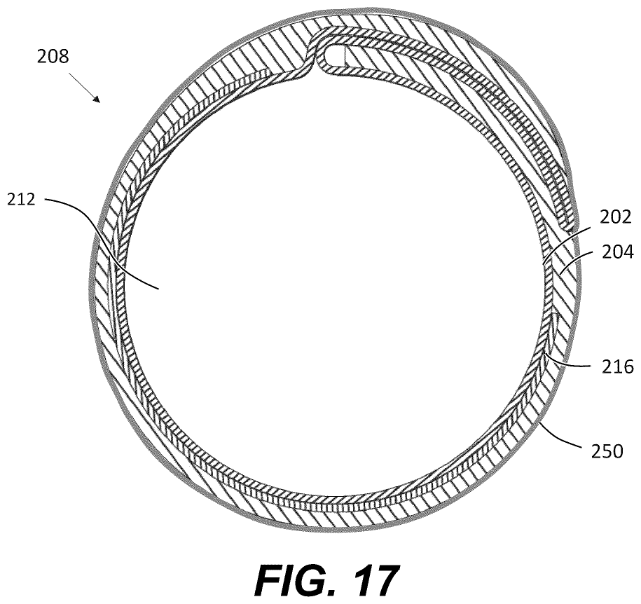

FIG. 17 shows a cross-section view of the sheath 8 taken near the distal end 210 of the sheath 8 as indicated by section line 17-17 in FIG. 16. As illustrated in FIGS. 16 and 17, the sheath 8 can include at least one radiopaque filler or marker, such as a discontinuous, or C-shaped, band (marker 216) positioned near the distal end 210 of the sheath 8. The marker 216 can be associated with the inner and/or outer layers 202, 204 of the sheath 8. For example, as shown in FIG. 17, the marker 216 can be positioned between the inner layer 202 and the outer layer 204. In alternative examples, the marker 216 can be associated with the outer surface of the outer layer 204. In some examples, the marker 216 can be embedded or blended within the inner layer 202 or outer layer 204.

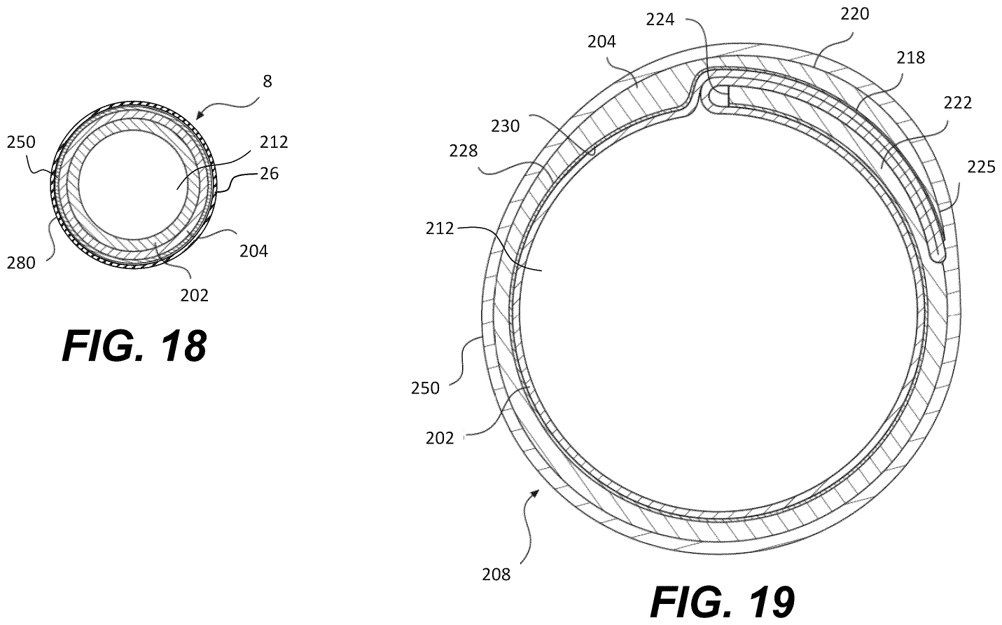

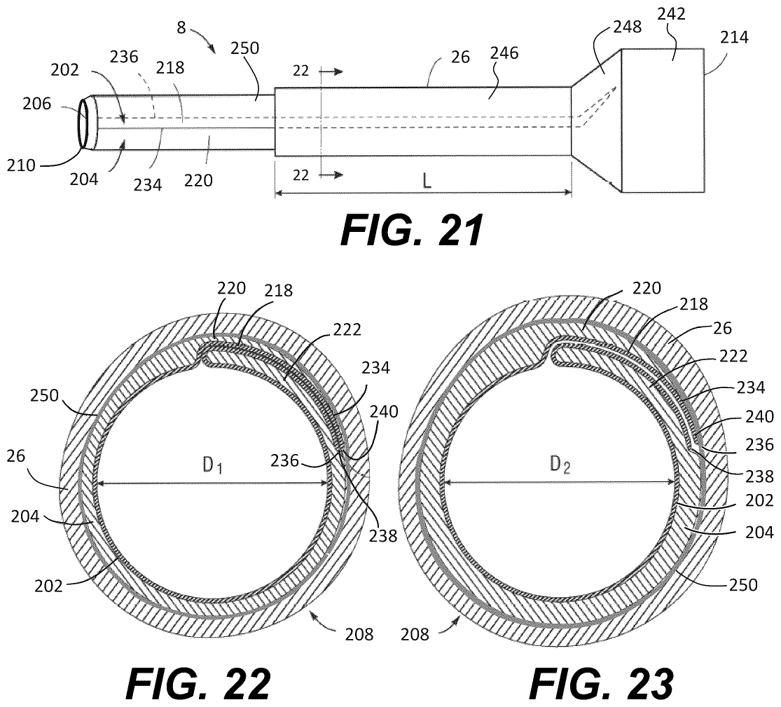

FIGS. 18 and 19 show additional cross sections taken at different points along the sheath 208. FIG. 18 shows a cross-section of a segment of the sheath near the proximal end 214 of the sheath 8, as indicated by section line 18-18 in FIG. 15. At this location, the sheath 8 includes the inner layer 202, outer layer 204, elastic outer layer 250/outer jacket, and the strain relief layer 26. At this location, near the proximal end of the sheath 8, the inner and outer layers 202, 204 are substantially tubular. Here the inner and outer layers 202, 204 can be formed without any slits or folded portions in the layers. By contrast, as described herein, the inner and outer layers 202, 204 at different locations along the sheath 8 (for example, at the point indicated by section line 19-19 in FIG. 15 and/or the point indicated by line section 22-22 in FIG. 21) can have a different configuration.

As shown in FIG. 19, the inner layer 202 can be arranged to form a substantially cylindrical lumen 212 therethrough. Inner layer 202 can include one or more folded portions 218. In the implementation shown in FIG. 19, inner layer 202 is arranged to have one folded portion 218 that can be positioned on either side of the inner layer 202. Inner layer 202 can be continuous, in that there are no breaks, slits, or perforations in inner layer 202. Outer layer 204 can be arranged in an overlapping fashion such that an overlapping portion 220 overlaps at least a part of the folded portion 218 of the inner layer 202. As shown in FIG. 19, the overlapping portion 220 also overlaps an underlying portion 222 of the outer layer 204. The underlying portion 222 can be positioned to underlie both the overlapping portion 220 of the outer layer 204, as well as the folded portion 218 of the inner layer 202. Thus, the outer layer 204 can be discontinuous, in that it includes a slit or a cut in order to form the overlapping portion 220 and underlying portion 222. In other words, a first edge 224 of the outer layer 204 is spaced apart from a second edge 225 of the outer layer 204 so as not to form a continuous layer.

As shown in FIG. 19, the sheath 8 can also include a thin layer of bonding or adhesive material 228 positioned between the inner and outer layers 202, 204. In one implementation, the adhesive material 228 can comprise a polyurethane material such as Tecoflex. The adhesive material 228 can be positioned on an inner surface of at least a portion of the outer layer 204 so as to provide adhesion between selected portions of the inner and outer layers 202, 204. For example, the outer layer 204 may only include a Tecoflex layer (adhesive material 228) around the portion of the inner surface 230 that faces the lumen-forming portion of the inner layer 202. In other words, the Tecoflex layer can be positioned so that it does not contact the folded portion 218 of the inner layer 202 in some implementations. In other implementations, the Tecoflex layer can be positioned in different configurations as desired for the particular application. For example, as shown in FIG. 19, the Tecoflex layer can be positioned along the entire inner surface 230 of the outer layer 204. In an alternative example, the Tecoflex layer can be applied to the outer surface of the inner layer 202 instead of the inner surface of the outer layer 204. The Tecoflex layer can be applied to all or selected portions on the inner layer 202; for example, the Tecoflex layer can be formed only on the portion of the inner layer 202 that faces the lumen-forming portion of the outer layer 204 and not on the folded portion 218. The configuration of FIG. 19 allows for radial expansion of the sheath 208 as an outwardly directed radial force is applied from within (for example, by passing a medical device such as a prosthetic heart valve through the lumen 212). As radial force is applied, the folded portion 218 can at least partially separate, straighten, and/or unfold, and/or the overlapping portion 220 and the underlying portion 222 of the outer layer 204 can slide circumferentially with respect to one another, thereby allowing the diameter of lumen 212 to enlarge.

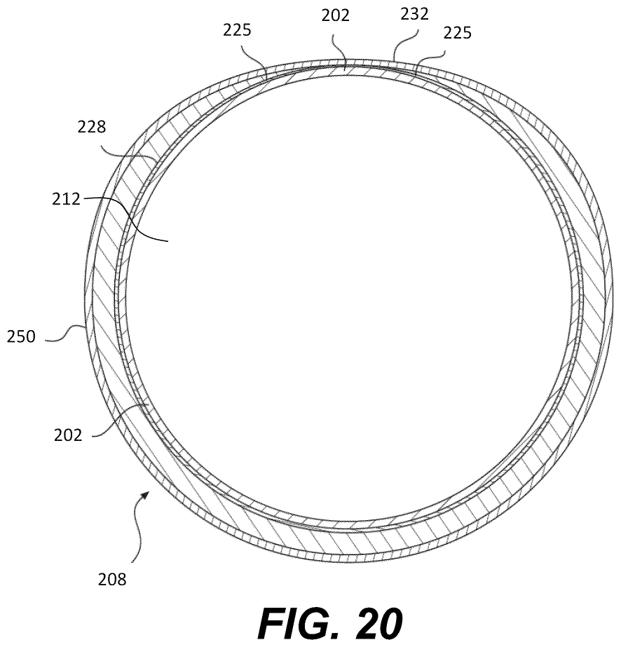

In this manner, the sheath 208 is configured to expand from a resting/unexpanded configuration (FIG. 19) to an expanded configuration shown in FIG. 20. In the expanded configuration, as shown in FIG. 20, an annular gap 232 can form between the longitudinal edges of the overlapping portion 220 and the underlying portion 222 of the outer layer 204. As the sheath 208 expands at a particular location, the overlapping portion 220 of the outer layer 204 can move circumferentially with respect to the underlying portion 222 as the folded portion 218 of the inner layer 202 unfolds. This movement can be facilitated by the use of a low-friction material for inner layer 202, such as PTFE. Further, the folded portion 218 can at least partially separate and/or unfold to accommodate a medical device having a diameter larger than that of lumen 212 in the resting/unexpanded configuration. As shown in FIG. 20, in some implementations, the folded portion of the inner layer 102 can completely unfold, so that the inner layer 102 forms a cylindrical tube at the location of the expanded configuration.

Similar to the example sheath 8 in FIG. 14, the sheath 8 is configured to locally expands at a particular location corresponding to the location of the medical device along the length of the lumen 212, and then locally contracts once the medical device has passed that particular location. Thus, a bulge may be visible, traveling longitudinally along the length of the sheath 8 as a medical device is introduced through the sheath 8, representing continuous local expansion and contraction as the device travels the length of the sheath 8. Each segment of the sheath 8 will locally contract after removal of any radial outward force such that the sheath 8 at least partially returns to the original resting/unexpanded diameter of lumen 212. Similar to the example sheath described herein, an elastic outer layer 250 can (optionally) be provided along the sheath 8, urging the inner and outer layers 202, 204 back towards the unexpanded configuration.

The inner and outer layers 202, 204 of sheath 8 can be configured having the folded portion 218 as shown in FIG. 19 along at least a portion of the length of the sheath 208. In some examples, the inner and outer layers 202, 204 can be configured as shown in FIG. 19 along the length A (FIG. 15) such that the folded portion 218 extends from a location adjacent the soft tip portion 206 to a location closer to the proximal end 214 of the sheath 8, adjacent and/or under the distal end of the strain relief layer 26. In this matter, the sheath 8 is expandable and contractable only along a portion of the length of the sheath corresponding to length A (which typically corresponds to the section of the sheath inserted into the narrowest section of the patient's vasculature).

In some examples, the folded portion 218 portion extends from a location adjacent the soft tip portion 206 under the strain relief layer 26, as illustrated in FIG. 21. In this example, the folded structure of the inner layer 202 extends from the soft tip portion 206, under the strain relief layer 26 and along the tapered portion 248 of the strain relief layer 26.

FIGS. 22 and 23 illustrate cross-section views of the sheath 8 taken along the strain relief layer 26 at section line 22-22 in FIG. 21. In this example, the folded portion 218 of the inner layer 202 extends under the strain relief layer 26. FIG. 22 shows a cross-section of the sheath 8 in a resting/unexpanded configuration having an inner diameter D₁. FIG. 23 shows a cross-section of the sheath 208 in a (partially) expanded configuration, having an inner diameter D₂, where D₂ is greater than D₁.

As shown in FIGS. 22-23, in some examples, the overlapping portion 220 does not overlap the entire folded portion 218 of the inner layer 202, and thus a portion of the folded portion 218 can be directly adjacent to the strain relief layer 26 in locations where the strain relief layer 26 is present. In locations where the strain relief layer 26 is not present, part of the folded portion 218 may be visible from the outside of the sheath 8, as seen in FIG. 21 (and/or visible through an elastic outer layer 250 described in more detail herein). In these examples, the sheath 8 can include a longitudinal seam 234 where the overlapping portion 220 terminates at the folded portion 218. In use, the sheath 8 can be positioned such that the seam 234 is posterior to the point of the sheath that is 180 degrees from the seam 234 (for example, facing downward in the view of FIG. 21). As shown in FIG. 21, the seam 234 need not extend the entire length of the sheath 8, and end at a transition point between portions of the sheath having a folded inner layer and portions of the sheath not having a folded inner layer.

In some examples, the folded portion 218 can include a weakened portion 236, such as a longitudinal perforation, score line, and/or slit, along at least a portion of the length of the inner layer 202. The weakened portion 236/slit allows for the two adjacent ends 238, 240 of the folded portion 218/inner layer 202 to move relative to one another as the sheath 8 expands to the expanded configuration shown in FIG. 23. For example, the sheath 8 locally expands as a medical device is inserted therethrough, causing the weakened portion 236 to split/separate.

In each of the example sheaths 8 described herein, the sheath 8 may include an clastic outer layer 250 that expands with the sheath 8. The elastic outer layer 250 can provide an inwardly directed radial force that directs the sheath towards a folded/unexpanded configuration. Similar to the strain relief layer 26, elastic outer layer 250 can also provide hemostasis (for example, prevent blood loss during implantation of the prosthetic device).

The elastic outer layer 250 can be positioned around at least a portion of the strain relief layer 26, outer layer 108, 204 and/or the inner layers of the sheath 8. As illustrated in FIGS. 21-23, the outer layer 250 can surround the entire circumference of outer layer 204, and can extend longitudinally along any portion of the length of the sheath 8, including along (over or under) the strain relief layer 26. The elastic outer layer 250 extends for a length along at least a portion of the main body of the sheath 8. In some examples, the elastic outer layer 250 extends to a point adjacent the distal end 210 or can extend all the way to the distal end 210 of sheath 8. For example, the elastic outer layer 250 extends over the entire length of the sheath 8.

As shown in FIGS. 17-20, 22 and 23, the elastic outer layer 250 can be a continuous tubular layer, without slits or other discontinuities. The elastic outer layer 250 extends between strain relief layer 26 and the outer surface of the outer layer 204. In other examples, the elastic outer layer 250 extends over the outer surface of the strain relief layer 26 and the outer surface of the outer layer 204. In further examples, the elastic outer layer 250 extends both over the strain relief layer 26 and/or between the outer layer of the sheath 8 and the strain relief layer 26.

The elastic outer layer 250 can comprise any pliable, elastic material(s) that expand and contract, preferably with a high expansion ratio. Preferably, the materials used can include low durometer polymers with high elasticity, such as Pebax, polyurethane, silicone, and/or polyisoprene. Materials for the elastic outer layer 250 can be selected such that it does not impede expansion of the inner and outer layers 202, 204 of the sheath 8. The elastic outer layer 250 can have a thickness ranging from, for example, about 0.001″ to about 0.010.″ In some implementations, the elastic outer layer 250 can have a thickness of from about 0.003″ to about 0.006.″ The elastic outer layer 250 can be configured to stretch and expand as the sheath expands, as shown in the expanded configuration in FIG. 20.

As illustrated in FIGS. 2, 15 and 21, the sheath 8 includes a strain relief layer 26. The strain relief layer 26/tube is provided adjacent the proximal end of the sheath 8 and extends along/over the outer surface of the sheath 8. In some examples, the strain relief layer 26 is provided over the outer layer 108, 204 of the sheath 8. The strain relief layer 26 forms a smooth transition between the sheath hub 20 and the sheath 8 and facilitates mating of the sheath 8 with the sheath hub 20.

Additionally, and as will be described in more detail herein, the strain relief layer 26 provides a region of higher durometer or stiffness that restricts expansion of the underlying sheath layers. This helps to ensure hemostasis between the portions of the sheath 8 inside the patient and the sheath hub (external to the patient). The increased durometer and/or stiffness along the strain relief layer 26 prevents blood from flowing between the various layers of the sheath 8 exterior to the patient during the procedure, helping to withstand the blood pressure that would otherwise cause the sheath to “balloon up” with body fluid/blood. Additionally, the strain relief layer 26 can be sized and configured to form a seal with the patient's artery when inserted, such that blood is substantially prevented from flowing between the strain relief layer 26 and the vessel wall. For example, although the strain relief layer 26 does not extend all the way to the distal end 210 of the sheath 8, the strain relief layer 26 can extend distally enough along the sheath 8 that when the sheath 8 is fully inserted into the patient a portion of the strain relief layer 26 extends through and seals against the arteriotomy site.

As described herein, the strain relief layer 26 is provided over the outer layer 108, 204 of the sheath 8. The strain relief layer 26 can be bonded to the outer layer 108, 204 to prevent the strain relief layer 26 from sliding over the outer layer and “bunching up” in response to the friction forces applied by the surrounding tissue during insertion of the sheath 8 into the patient's vasculature. For example, the strain relief layer 26 can be bonded at the proximal end and/or distal end of the outer layer 108, 204. At the proximal and distal ends, the strain relief layer 26 can be bonded to the outer layer 204 around the full circumference of the outer layer. At the distal end of the sheath 208, the strain relief layer 26 can alternatively be bonded to the inner layer(s) of the sheath 8. For example, the strain relief layer 26 can be bonded to the distal end surface of the inner layer 102, 202.

FIGS. 18, 22 and 23 illustrate cross-section views of the sheath 8 along the strain relief layer 26. FIG. 18 shows a cross-section of a segment of the sheath near the proximal end 214 of the sheath 8, as indicated by line 18-18 in FIG. 15. Similarly, FIGS. 22 and 23 show cross-section segments of various example sheaths near the proximal end 214 of the sheath 8 and closer to the distal end of the strain relief layer 26, as indicated by section line 22-22 in FIG. 21. As illustrated in each of FIGS. 15-23, the sheath 8 at this location can comprise an inner layer (liner) 202, outer layer 204, adhesive material 228 layer, an optional elastic outer layer 250, and the strain relief layer 26.

The strain relief layer 26 extends circumferentially around at least a portion of the inner layer 202 and outer layer 204. The strain relief layer 26 extends from the proximal end 214 of the sheath 8 towards the distal end 210 of the sheath 8. As shown in FIG. 21 (and FIG. 15), the strain relief layer 26 extends for a length L along at least a portion of the main body of the sheath 8. In further examples, the strain relief layer 26 extends to a point adjacent the distal end 210, or can extend all the way to the distal end 210 of sheath 8. In some examples, the longitudinal length L of the strain relief layer 26 can range from about 10 cm to the entire length of the sheath 8.

The strain relief layer 26 extends to/adjacent the proximal end 214 of the sheath 8 and provides a compression fit over the distal end of the sheath hub 20 thereby coupling the sheath 8 to the sheath hub 20. Additionally, or alternatively, the strain relief layer 26 secured between the sheath hub 20 and the sheath hub cap 22 or other fastening device for by coupling the proximal end of the sheath to the sheath hub 20. In some examples, the strain relief layer 26 does not extend all the way to the proximal end 214 of the sheath 208.

It is understood that strain relief layer 26, as shown herein, can have similar composition and characteristics of the inner and outer layers as disclosed herein. Various compositions are disclosed, for example, in Application No. PCT/US2021/301275, entitled “Expandable sheath for introducing an endovascular delivery device into a body,” the disclosure of which is herein incorporated by reference.

The strain relief layer 26 can comprise any lubricious, low-friction, and/or relatively non-elastic material. Preferably the materials used can include high durometer polymers, with low elasticity. In some examples, the strain relief layer 26 is composed of the same and/or similar material to the inner layer 202 and/or outer layer 204. For example, as described herein regarding the inner layer 102 and/or outer 108, exemplary materials can include polyurethane (for example, high density polyethylene), ultra-high-molecular-weight polyethylene (UHMWPE) (for example, Dyneema®), high-molecular-weight polyethylene (HMWPE), or polyether ether ketone (PEEK). Other suitable materials strain relief layer 26 can include polyimide, polytetrafluoroethylene (PTFE), expanded polytetrafluoroethylene (cPTFE), ethylene tetrafluoroethylene (ETFE), nylon, polyethylene, polyamide, polyether block amide (for example, Pebax), and/or combinations of any of the materials described herein. Materials for the strain relief layer 26 can be selected such that it impedes expansion of the underlying layers of the sheath 8.

The strain relief layer 26 can have a thickness ranging from, for example, about 0.001″ to about 0.010.″ In some implementations, the strain relief layer 26 can have a thickness of from about 0.003″ to about 0.006.″ The wall thickness is measured radially between the inner surface of the strain relief layer 26 and the outer surface of the strain relief layer 26.

In alternative examples, the material composition and/or wall thickness can change along the length of the strain relief layer 26. For example, the strain relief layer 26 can be provided with one or more segments, where the composition and/or thickness changes from segment to segment. In an example aspect, the Durometer rating of the composition changes along the length of the strain relief layer 26 such that segments near the proximal end comprise a stiffer material or combination of materials, while segments near the distal end comprise a softer material or combination of materials. Similarly, the wall thickness of the strain relief layer 26 in segments near the proximal end can be thicker/greater than the wall thickness of the elastic outer layer 250 near the distal end.

As illustrated in FIGS. 15, 21 and 24, the strain relief layer 26 has a proximal end and a distal end and a central lumen extending longitudinally therethrough. The strain relief layer 26 includes a generally tubular shaped proximal portion 242 adjacent the proximal end of the strain relief layer 26, and a generally tubular shaped distal portion 246 adjacent the distal end of the strain relief layer 26. The strain relief layer 26 includes a frustoconical shaped tapered portion 248 extending between the proximal portion 242 and the distal portion 246 of the strain relief layer 26, such that the diameter of the strain relief layer 26 at the proximal portion 242 is greater than the diameter of the strain relief layer 26 at the distal portion 246 of the strain relief layer 26. The tapered portion 248 and the flared proximal portion 242 help case the transition of the medical device/delivery system when passing between the larger diameter sheath hub 20 to the smaller diameter of the sheath 8.

As described herein, the strain relief layer 26 is made of a material that is stiffer than the other sheath 8 layers such that the strain relief layer 26 inhibits expansion of the portion of the sheath disposed along/under the strain relief layer 26. Because radial expansion is limited along the strain relief layer 26, higher push forces are necessary to advance the medical device (implant 12) through the central lumen of the sheath 8. In some examples, the highest push force through the sheath 8 are experienced near the ends (for example, proximal and distal ends) of the strain relief layer 26.

In some aspects, the thickness and/or composition of the strain relief layer 26 can be adjusted to improve the performance of the strain relief layer 26 and to reduce the push force.

As described herein, pre-dilating the sheath 8, or a portion thereof, can help to reduce push forces required to insert the medical device/delivery system through the central lumen of the sheath 8. Pre-dilating the sheath 8 releases and/or loosens any bonding or adhesion of the sheath 8 layers that occurs during the manufacturing process, for example, bonding between the inner and outer layers 202, 204, bonding between the folded portion 218 and outer layer 204, bonding between the inner/outer layers 202, 204 and the strain relief layer 26, bonding or resistance to expansion along the strain relief layer 26 or at the distal tip. Pre-dilating can also break or separate the weakened portion 236 of folded portion 218 of the inner layer 202, separating adjacent ends 238, 240 of the folded portion 218, as described herein and illustrated in FIG. 23. With the sheath 8 layers able to move freely with respect to the other, the medical device/delivery system is pushed through the sheath 8 lumen at a much lower force.

In some instances, the sheath 8 is pre-dilated by passing a relatively large dilator (for example, 22 French dilator) through the sheath 8, including the strain relief layer 26 and/or distal tip. This can be done during sheath 8 preparation, prior to sheath 8 insertion into the patient and/or with the sheath 8 at least partially inserted into the patient. However, this method requires significant physical strength of the user (i.e., grip and arm strength) to advance the dilator into the strain relief layer 26 and/or through the distal tip. Additionally, in some instances it is desirable to control the length of the sheath that is expanded. It may be important that the dilator not pass significantly beyond the distal end of the strain relief layer 26 to avoid splitting the main body of the sheath 8 beyond the distal end of the strain relief layer 26. Depending on the sheath structure, expanding/dilating the portion of the sheath 8 beyond the end of the strain relief layer 26 can cause irregular sheath 8 expansion as the expanded portion of the sheath 8 may not recover smoothly to/toward the original unexpanded configuration and this results in difficulty or vessel injury during insertion, movement and/or withdraw of the pre-dilated sheath 8 in the vasculature. In some implementations, it may be desirable to pre-dilate the entire length of the sheath 8, including the distal tip portion. Similar to the strain relief layer 26, the distal tip of the sheath 8 resists radial expansion and requires increased push force to advance the delivery device beyond and/or through the distal opening of the sheath 8.

Current methods for controlling dilation length of the sheath 8 and/or strain relief layer 26 are prone to user error and/or inaccuracies because they rely on a user's visual observation of the dilator as it passes through the sheath 8. These manual methods are inherently difficult to train, difficult to enforce proper technique, and prone to errors.

The example sheath system described herein, provides a solution to remove human error, ensuring that the desired length of the sheath 8 is pre-dilated. The system described herein allows the sheath 8 to be dilated after insertion into the patient and/or with the sheath 8 partially inserted into the patient. This system eliminates the need to dilate the sheath 8 during preparation, reduces preparation time, and eliminates unnecessary opportunities for error and/or inadvertent damage to the sheath 8.

The devices, systems, and methods described herein provide for a system including an expandable dilator/introducer that can be quickly and accurately used to dilate a desired length of the sheath 8, including the entire length from the distal tip to the strain relief layer 26.

FIGS. 24-71 show an example expandable introducer system 300. The introducer system 300 includes an elongated introducer body 310. An elongated core member 350 is received (and movable) within the central lumen 312 of the introducer body 310. As will be described in more detail herein, the introducer body 310 can be inserted into the sheath 8 in an unexpanded configuration. As the core member 350 moves within the central lumen 312 of the introducer body 310, interference between the core member 350 and the central lumen 312 causes the introducer body 310 to radially expand. The expanded introducer system 300 can then be advanced/withdrawn within the central lumen of sheath 8 expanding/dilating the desired length and portions of the sheath 8.

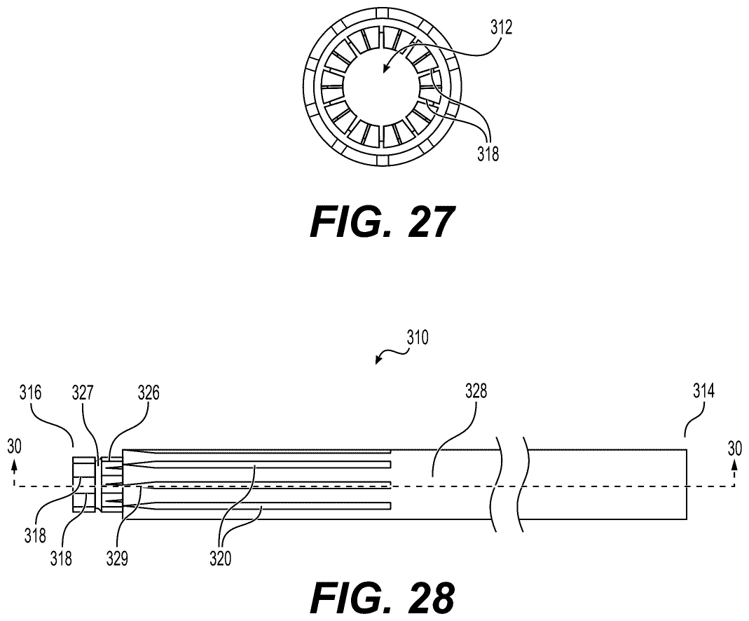

FIGS. 24-26 illustrate the introducer body 310 and corresponding slot sleeve 370. FIGS. 27-33 provide various views of the introducer body 310. The introducer body 310 includes an elongated generally cylindrical elongated main body portion 328. A central lumen 312 extends between a proximal end 314 and distal end 316 of the introducer body 310. The introducer body 310 includes slits 318 and slotted openings 320 positioned circumferentially around the introducer body 310 that allow for the introducer body 310 to radially expand.

As illustrated in FIG. 28, the introducer body 310 includes a plurality of slits 318 extending axially from the distal end of the introducer body 310 toward a proximal end of the introducer body 310. The slits 318 allow for the introducer body 310 to couple to the distal introducer tip 330, while also allowing the adjacent portion of the introducer body 310 to radially expand. FIG. 27 provides a distal end view of the introducer body 310. The plurality of slits 318 are evenly spaced around the circumference of the introducer body 310. In some implementations, the introducer body 310 includes a single slit 318. In further implementations, the introducer body 310 includes from two to 20 slits 318. As shown in FIG. 27, the introducer body 310 can include 10 slits 318. In some aspects, the slits 318 extend through the entire wall thickness of the introducer body 310. In further aspects, the slits 318 extends at least partially through a wall thickness of the introducer body 310. For example, the slits 318 can be provided by a weakened portion, such as a longitudinal perforation, score line, and/or slit, allowing the slits 318 to flex and stretch, and separate if needed.

As illustrated in FIG. 28, the distal end 316 of the introducer body 310 can include a reduced diameter recessed shoulder 326. The recessed shoulder 326 extends from the distal end 316 of the introducer body 310 toward the proximal end 314 of the introducer body 310. The recessed shoulder 326 has a smaller diameter than the main body portion 328 of the introducer body 310 and is sized and configured to be received within a central lumen 332 of the distal introducer tip 330. In some aspects, the slits 318 are provided on a recessed shoulder 326 extending from the distal end 316 of the introducer body 310. In some examples, as shown in FIG. 28, the slits 318 extend along a majority of the length of the recessed shoulder 326. In further examples, the slit extends along the entire length of the recessed shoulder 326.

In some aspects, the width of the slits 318 is constant along an entire length of a particular slit 318 and/or between various slits 318. In other aspects, the width of the slits 318 vary along a length of the slit 318 and/or between various slits 318.

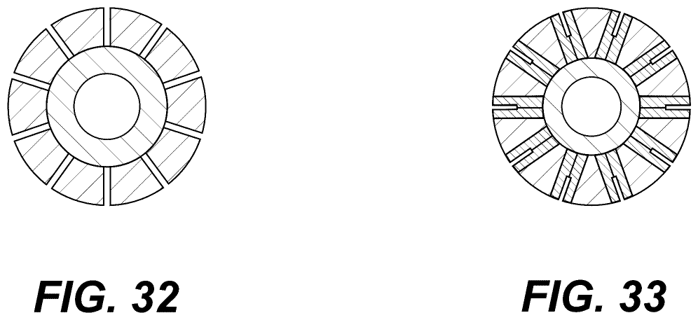

As provided in FIG. 28, the introducer body includes a plurality of slotted openings 320 extending along a portion of the introducer body 310. The slotted openings 320 allow the corresponding portion of the introducer body 310 to radially expand. The plurality of slotted openings 320 are evenly spaced around the circumference of the introducer body 310 (for example, FIG. 32). In some implementations, the introducer body 310 includes a slotted opening 320. In further implementations, the introducer body 310 includes from two to 20 slotted openings 320. As shown in FIG. 32, the introducer body 310 includes can include 10 slotted openings 320. In some aspects, the slotted opening 320 extends through the entire wall thickness of the introducer body. In further aspects, the slotted opening 320 extends at least partially through an entire wall thickness of the introducer body 310. For example, the slotted openings 320 can be provided by a weakened portion, such as a longitudinal perforation, score line, and/or slit, allowing the slotted openings 320 to flex and stretch and separate if needed.

As illustrated in FIGS. 28 and 30, the slotted openings 320 extend along a portion of the introducer body 310 between the proximal and distal ends 314, 316. For example, as shown in FIG. 28, the slotted openings 320 are provided proximate the distal end 316 of the introducer body 310, but do not extend to the distal end 316 of the introducer body 310. Rather, the slotted openings 320 are provided primarily on the main body portion 328 of the introducer body 310. A portion of the slotted opening 320 may extend along a portion of the recessed shoulder 326, allowing the introducer body 310 to expand along its distal end and along a portion of the shoulder 326. As provided in FIGS. 28 and 30, the slits 318 are provided on the recessed shoulder 326 extending from the distal end 316 of the introducer body 310, and a portion of the length of the slits 318 axially overlap with a portion of the length of the slotted openings 320 along the shoulder 326.

In some aspects, the width of the slotted openings 320 is constant along an entire length of a particular slotted opening 320 and/or between various slotted openings 320. In other aspects, the width of the slotted openings 320 tapers at a distal end 329 of the slotted opening 320 (FIGS. 28 and 30). The tapered width allows the slotted openings 320 to expand/widen as the introducer body 310 is expanded into an expanded configuration.

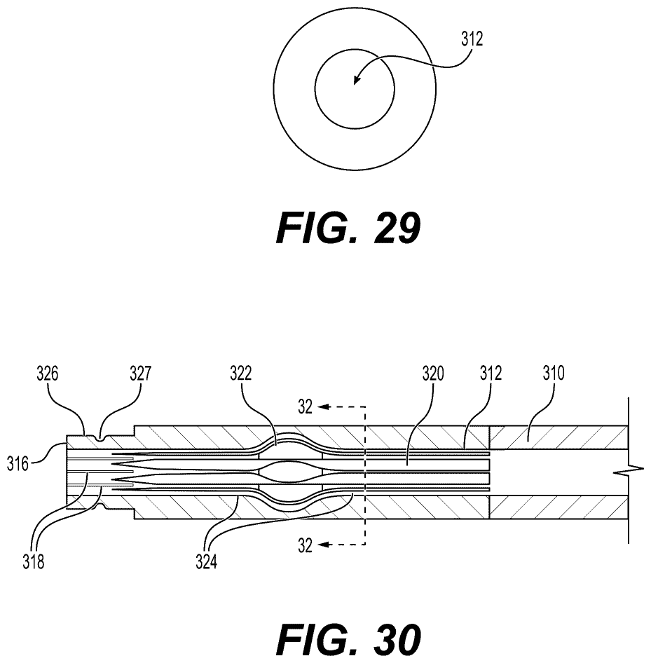

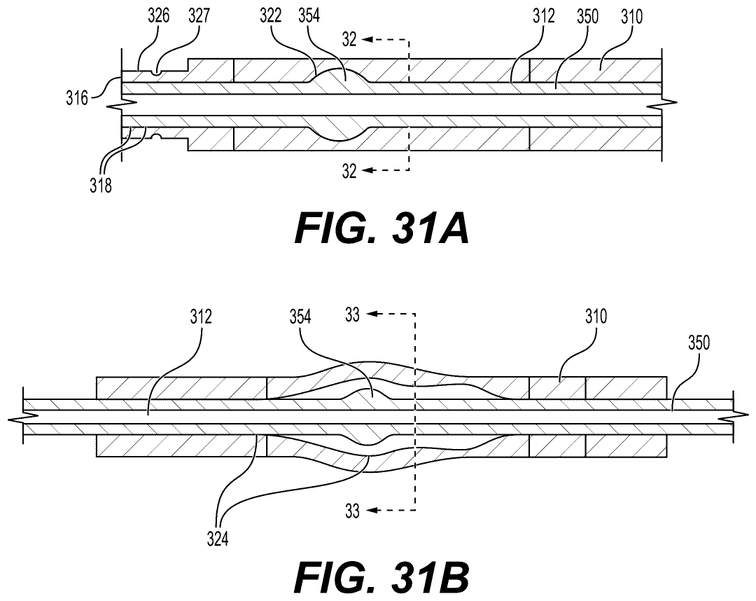

FIGS. 27 and 29 provide distal and proximal end views, respectively, of the elongated introducer body 310. FIG. 30 provides an axial cross-sectional view of the introducer body 310 taken along section line 30-30 in FIG. 28. FIGS. 31A and 31B provide axial cross-sectional view of the introducer body 310 and core member 350 taken along section line 30-30 in FIG. 28. FIG. 31A shows the introducer body in the unexpanded configuration, and FIG. 31B shows the introducer body in the expanded configuration. Similarly, FIG. 32 provides a (radial) cross-sectional view of the unexpanded introducer body 310 taken along section line 32-32 in FIG. 30, and FIG. 33 provides a (radial) cross-sectional view of the expanded introducer body 310 taken along section line 33-33 in FIG. 31. As illustrated in FIGS. 27, 29, and 30-33, the introducer body 310 includes a central lumen 312 extending between the proximal end 314 and the distal end 316 of the introducer body 310. The central lumen 312 of the introducer body 310 is sized and configured to receive the core member 350, described in more detail herein. At least a portion of the introducer body 310 is configured to expand from an unexpanded configuration, shown in FIGS. 30, 31A and 32, in which the introducer body 310 has a first outer diameter, to an expanded configuration, shown in FIGS. 31B and 33, in which the introducer body 310 has a second, larger, outer diameter. The introducer body 310 expands in response to an outwardly directed radial force exerted on the central lumen 312 of introducer body 310 by the core member 350 as it moves between an unexpanded position and expanded position (FIG. 31) with respect to the introducer body 310.

As will be described in more details herein, core member 350 includes an elongated body portion 352 and an expansion element 354 extending radially from the outer surface 356 of the body portion 352. Contact between the expansion element 354 and the central lumen 312 of the introducer body 310 provides the outwardly directed radial force on the central lumen 312 for directing expansion of the introducer body 310.

Illustrated in FIGS. 30, 31A and 31B, central lumen 312 of the introducer body 310 includes a cavity 322 for receiving the expansion element 354. The cavity 322 has a size and shape corresponding to the size and shape of the expansion element 354, such that when the core member 350 is in the unexpanded position (FIG. 31A) it is received within the cavity 322 and does not exert an outwardly directed radial force on the central lumen 312 of the introducer body, and the introducer body 310 is in the unexpanded configuration. As provided in FIG. 30, the cavity 322 has a diameter greater than a diameter of a main portion 324 of the central lumen 312. In some aspects, the cavity 322 includes a tapered proximal edge for reducing pull forces required to move the core member 350 distally within the central lumen 312. When the core member 350 is moved to the expanded position (FIG. 31B), the core member 350 is moved axially within the central lumen 312 and the expansion element is moves from the cavity 322 to the main portion of the central lumen 312, thereby causes the introducer body 310 to expand toward/to the expanded configuration. As illustrated in FIG. 30, the cavity 322 is provided within the central lumen 312 at a location corresponding to the slotted openings 320. Accordingly, the portion of the introducer body 310 including the slotted opening 320 defines the portion of the introducer body 310 that is movable between the unexpanded and the expanded configuration.

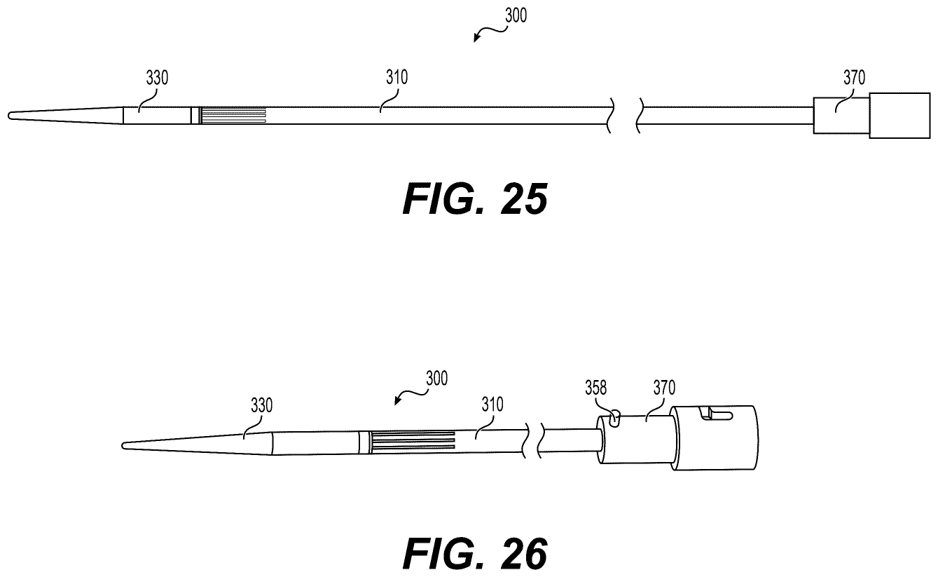

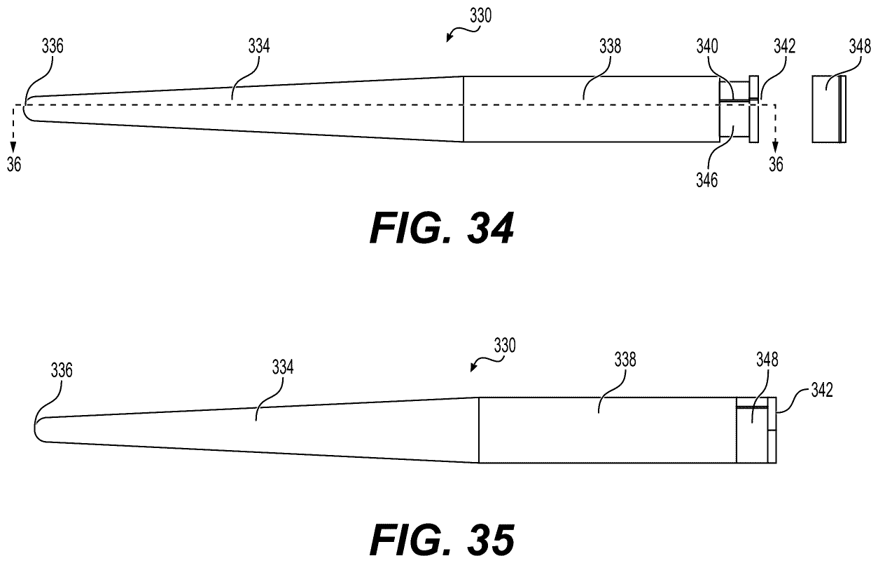

As provided in FIGS. 25-26, the introducer body 310 includes a distal introducer tip 330 coupled to its distal end 316. As illustrated in FIGS. 34-36, the introducer tip 330 includes a tapered distal end portion 334 to assist with advancing the introducer system 300 into the patient's vasculature. The tapered distal end portion 334 tapers from a distal end 336 of the introducer tip 330 to a main body portion 338 of the introducer tip 330. For example, the main body portion 338 includes a constant diameter portion extending from the tapered distal end portion 334 toward the proximal end 342 of the introducer tip 330. The tapered distal end portion 334 and the main body portion 338 provide a smooth and tight fit between the introducer body 310 and the distal opening of the sheath 8 when the introducer system 300 is received within the central lumen of the sheath 8.

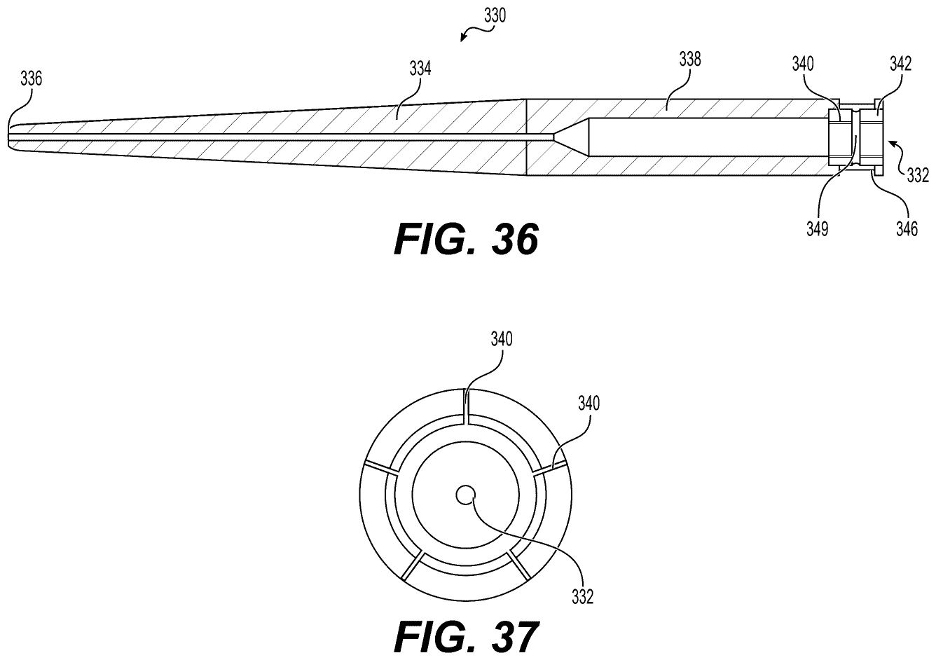

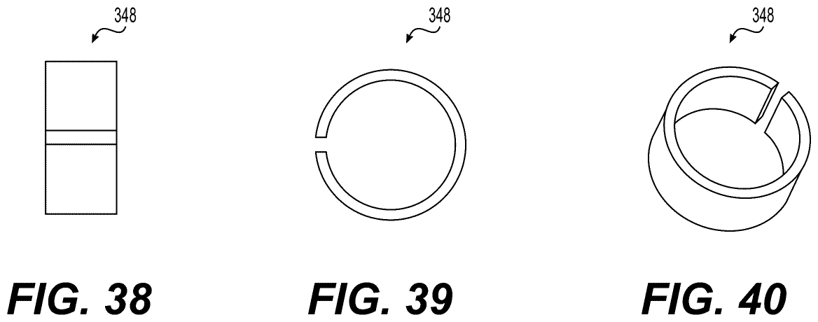

Illustrated in FIGS. 34-36, the introducer tip 330 includes a slit 340 extending (axially/longitudinally) from the proximal end 342 of the introducer tip 330 toward the distal end 336 of the introducer tip 330. The slit 340 allows the proximal end of the introducer tip 330 to compress/flex around the distal end/recess shoulder 326 of the introducer body 310. The introducer tip 330 for securing the introducer tip 330 to the introducer body 310. In some implementations, the introducer tip 330 includes a plurality of slits 340 evenly spaced around the circumference of the introducer tip 330. In some implementations, the introducer tip 330 includes a single slit 340. In further implementations, the introducer tip 330 includes from two to 10 slits 340. As shown in FIG. 37, the introducer tip 330 can include five slits 340. In some aspects, the slits 340 extend through the entire wall thickness of the introducer tip 330. In further aspects, the slits 340 extend at least partially through a wall thickness of the introducer tip 330. For example, the slits 340 can be provided by a weakened portion, such as a longitudinal perforation, score line, and/or slit, allowing the slits 340 to flex and stretch, and separate if needed. As provided in FIGS. 34 and 36, the introducer tip 330 includes an annular groove 346 sized and configured to receive a retaining ring 348. As illustrated in FIG. 35, when the introducer body 310 is received within the central lumen 332 of the introducer tip 330, the retaining ring 348 is provided over the outer surface of the introducer tip 330 within the annular groove 346 such that radially inward force provided by the retaining ring 348, compresses the introducer tip 330 between the retaining ring 348 and the introducer body 310, securing the introducer tip 330 to the introducer body 310. An example retaining ring 348 is illustrated in FIGS. 38-40. In some examples, the retaining ring 348 includes a stainless-steel retaining ring, continuous or discontinuous ring/clip. In some implementations, the introducer tip 330 includes a tip engagement feature 349 provided on the central lumen 312. The engagement feature 349 is sized and configured to engage a corresponding body engagement feature 327 extending circumferentially around the recessed shoulder 326 of the introducer body 310. In some examples, the body engagement feature 327 provided on the recessed shoulder 326 includes an annular recess extending circumferentially around the recessed shoulder 326, and the corresponding tip engagement feature 349 includes a correspondingly shaped projection extending radially from the central lumen 332 of the introducer tip 330.

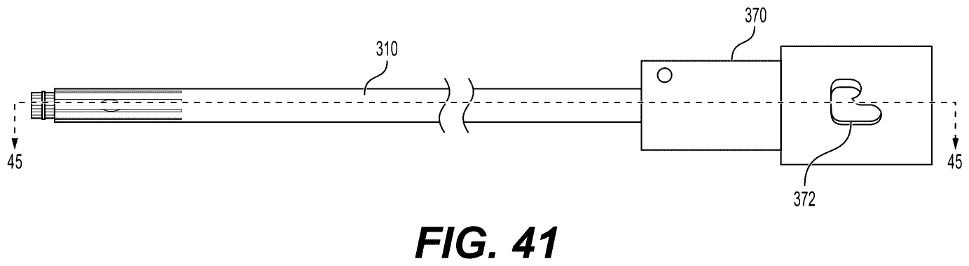

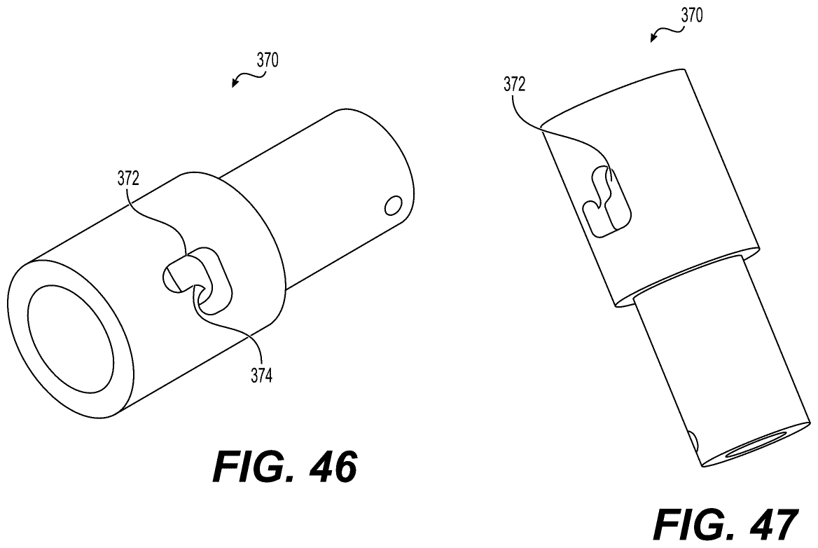

As illustrated in FIGS. 25 and 26, the proximal end 314 of the introducer body 310 is coupled to a slot sleeve 370. As will be described in more detail herein, the slot sleeve 370 guides movement of the core member 350 within the introducer body 310. FIGS. 41-47 illustrate an example slot sleeve 370. The slot sleeve 370 includes a guide slot 372 for receiving a traveling pin 358 provided on the core member 350 (FIG. 61). The traveling pin 358 is movable within the guide slot 372 as the core member 350 moves between the unexpanded and expanded positions. In some implementations, as shown in FIG. 43, the guide slot 372 is generally L-shaped, including an axially-extending proximal portion allowing the core member 350 to move axially with respect to the introducer body 310, and a circumferentially-extending distal portion allowing the core member 350 to rotate with respect to the introducer body 310/slot sleeve 370. The guide slot 372 includes a catch 374 for maintaining the traveling pin 358 in the expanded position, as illustrated in FIGS. 46-47.

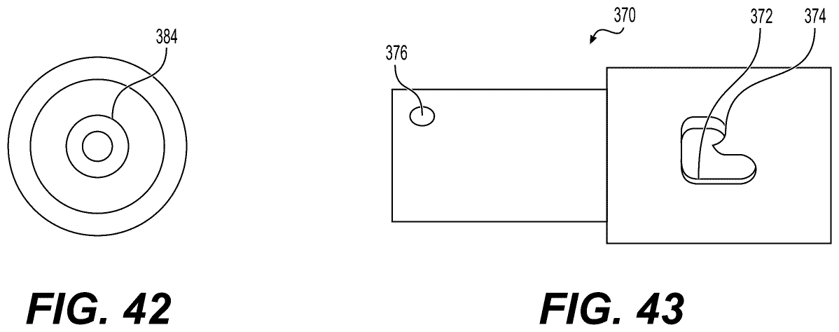

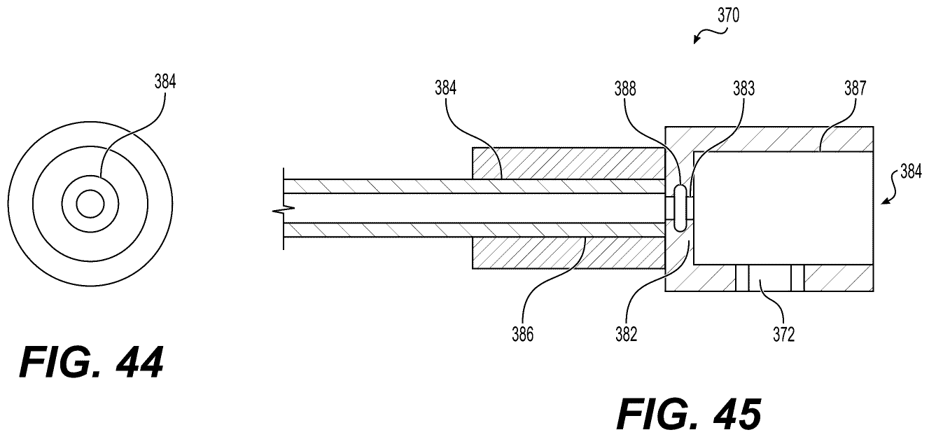

As provided in FIGS. 42 and 44, the slot sleeve 370 includes a central lumen 384. As illustrated in FIG. 45, the central lumen 384 of the slot sleeve 370 includes a reduced diameter distal portion 386 and a large diameter proximal portion 387 separated by an inner shoulder 382. The introducer body 310 is received within the reduced diameter distal portion 386 of the central lumen 384 of the slot sleeve 370. In some examples, the slot sleeve 370 is fixedly coupled to the proximal end 317 of the introducer body 310. For example, in some implementations, the introducer body 310 is fixedly coupled to the slot sleeve 370 by, for example, a mechanical fastener (for example, a press fit, an interference fit, a snap fit, a pin, thread, bayonet fastener, clip, locking key), a chemical fastener (for example, an adhesive, a UV bonded adhesive), a thermal process (for example, a weld, a reflow process), and/or any other suitable coupling process known in the art).

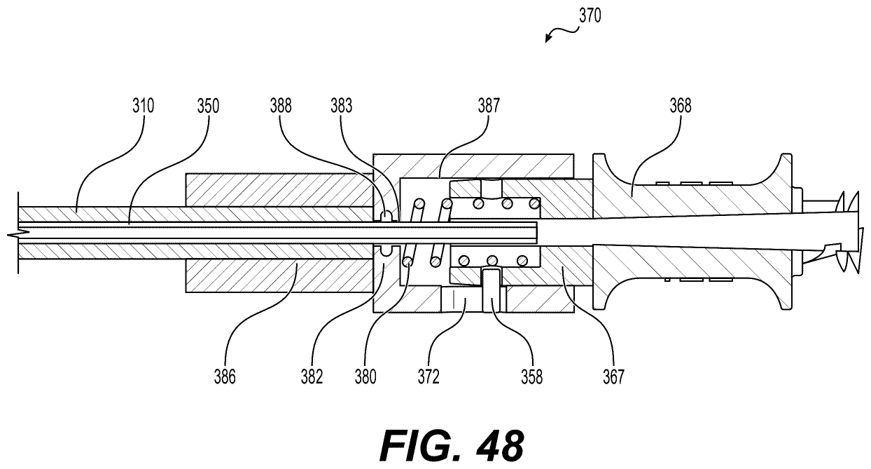

As illustrated in FIG. 48, the core member 350/core member hub 368 is received within the large diameter proximal portion 387 of the slot sleeve 370. In this way, the slot sleeve 370 operably couples introducer body 310 with the core member 350 and controls the axial and rotational movement therebetween. The core member 350 is received and movable (axially and/or rotationally) within the central lumen 312 of the introducer body 310, the central lumen 384 of the slot sleeve 370 (including the opening 383 provided in the inner shoulder 382), and the central lumen 332 of the introducer tip 330. The body portion 352 of the core member 350 passes through the opening 383 provided in the inner shoulder 382 of the slot sleeve 370 and into the central lumen 312 of the introducer body 310. The inner shoulder 382 includes a cavity 388 sized and configured to receive a seal/o-ring to prevent fluid back-flow from leaking between the core member 350 and the slot sleeve 370.

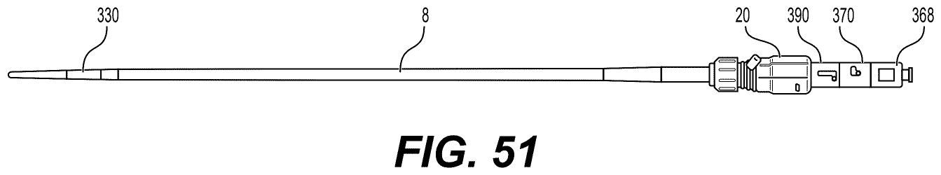

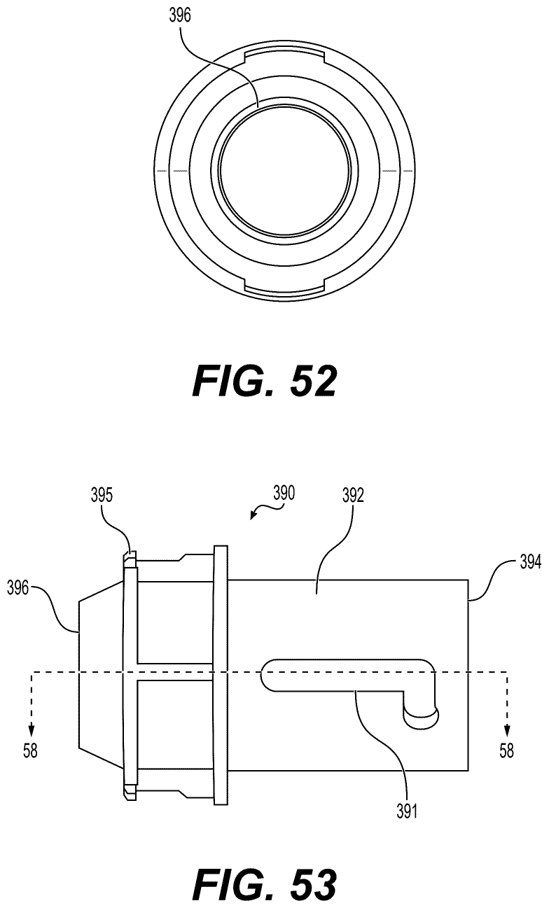

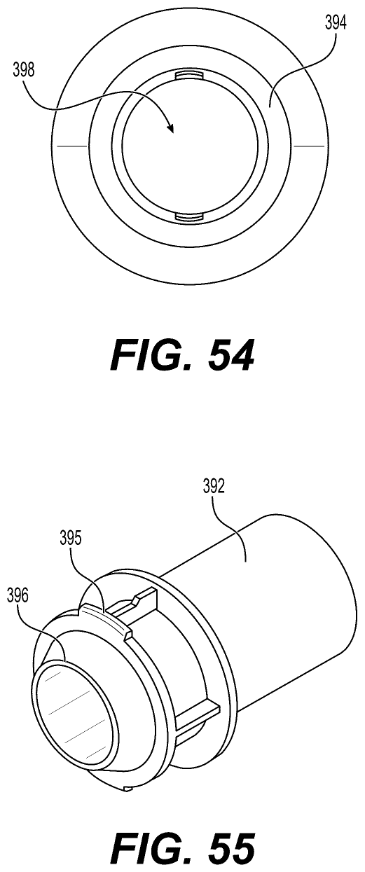

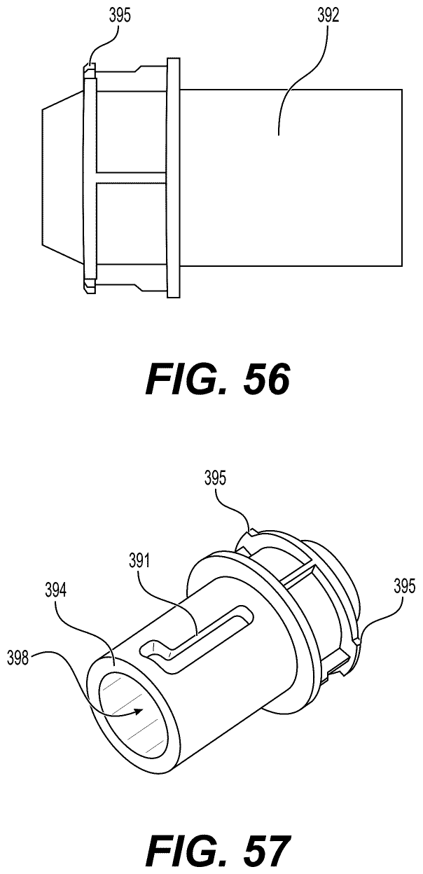

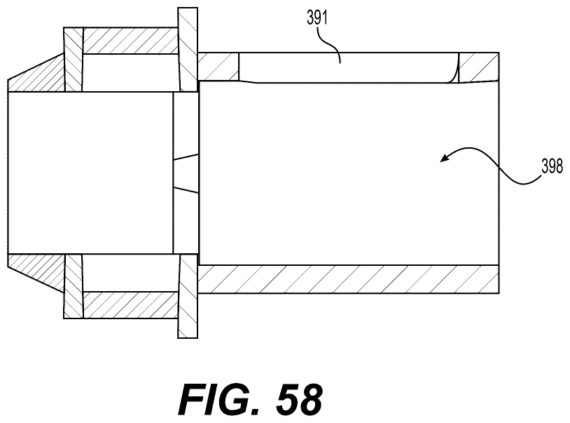

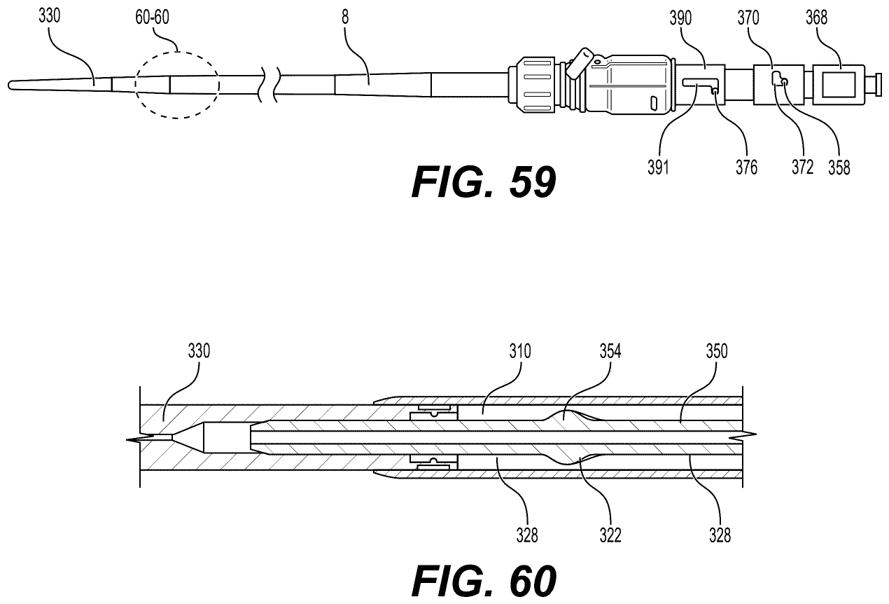

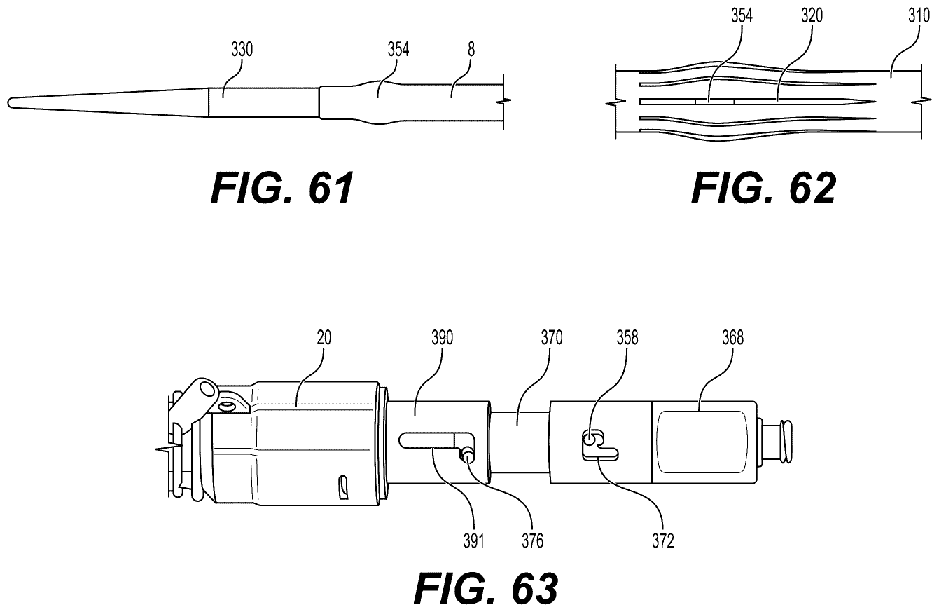

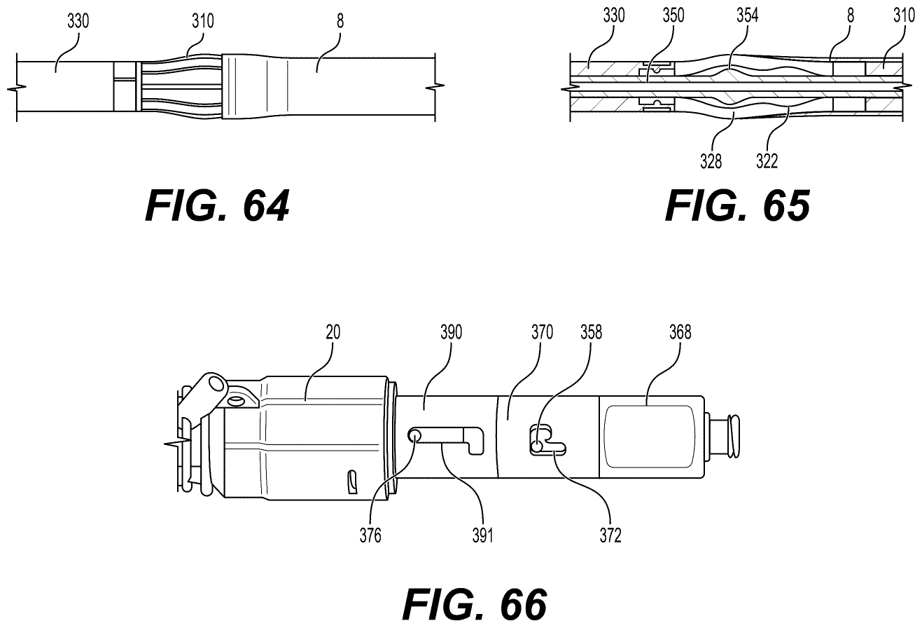

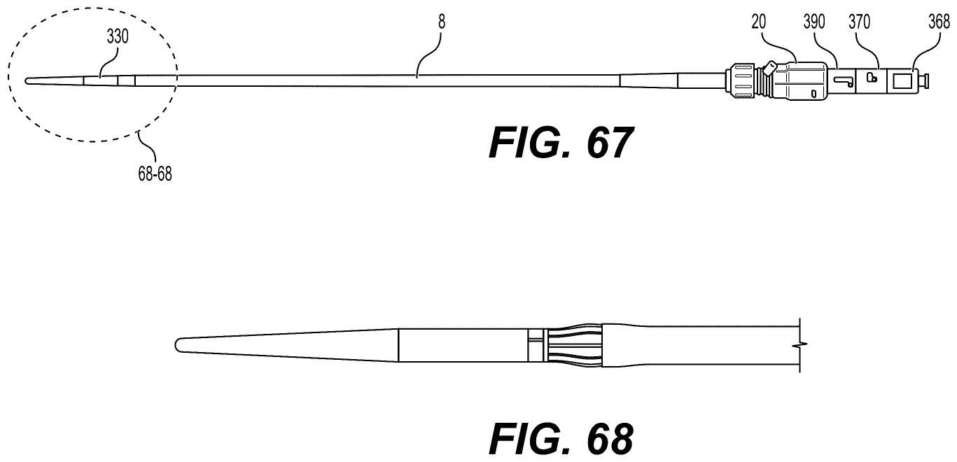

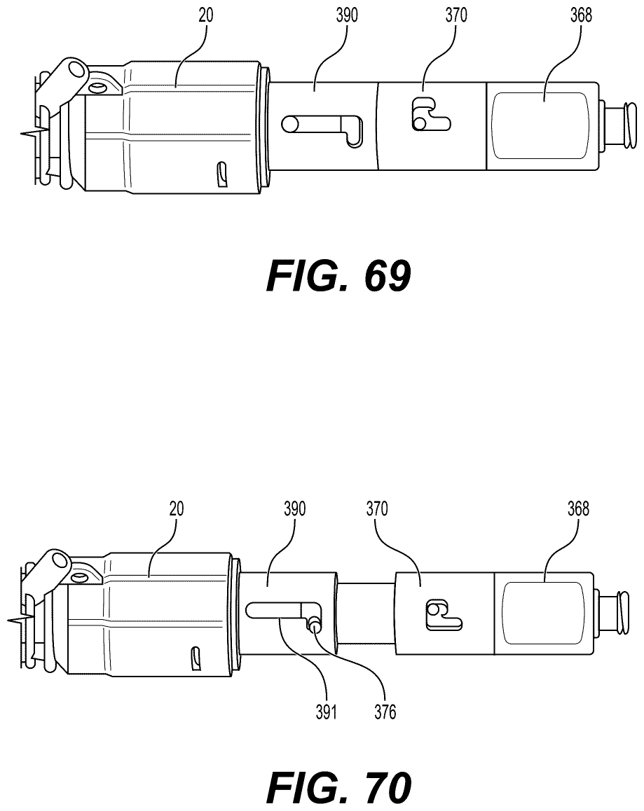

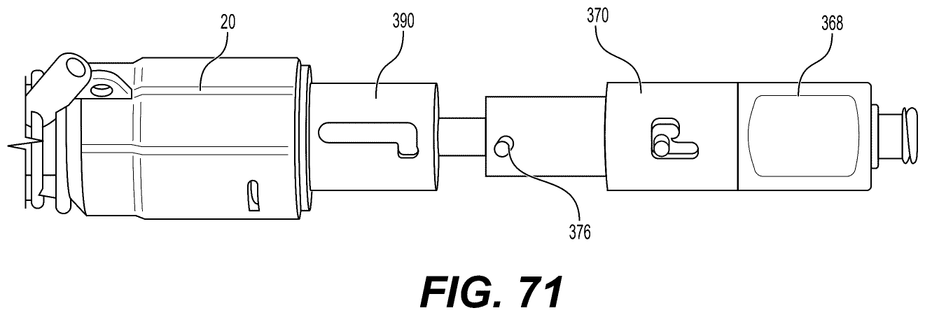

FIGS. 49 and 50 illustrate an example core member 350. The core member 350 includes an elongated body portion 352 and an expansion element 354 extending radially from the outer surface 356 of the elongated body portion 352. In some examples, the expansion element 354 includes a regular or irregular shaped projection extending from the outer surface 356 of the elongated body portion 352. In some aspects, the expansion element 354 extends around all or a portion of the circumference of the elongated body portion 352. As illustrated in FIG. 50, expansion element 354 has a circular shape in cross-section. In some implementations, the diameter of the expansion element 354 ranges from 12 Fr to 22 Fr. In further implementations, the expansion element 354 has a diameter ranging from 14 Fr to 22 Fr. In a further aspect, the expansion element 354 has a diameter of 22 Fr (for example, 7.3 mm, 0.288 inches). The main portion 324 of the diameter of the central lumen 312 when expanded by the expansion element 354 ranges from 12 Fr to 34 Fr. In some examples, the diameter of the central lumen 312 when expanded by the expansion element 354 is at least 24 Fr.

In some aspects, the core member body portion 352 has a length corresponding to or less than the length of the introducer body 310. In further aspects, the core member body portion 352 has a length greater than the introducer body 310, such that the distal end 362 extends beyond the distal end 316 of the introducer body 310 and into the central lumen 332 of the introducer tip 330, for example, when the core member hub 368 is coupled to the slot sleeve 370. As illustrated in FIGS. 49 and 50, the core member body portion 352 includes a tapered distal end 362.

The proximal end 364 of the core member 350 is coupled to a core member hub 368. In some aspects, the core member hub 368 includes a luer hub. The core member 350 includes a central lumen 360 extending therethrough (for example, for receiving a guidewire). As illustrated in FIG. 50, the central lumen 360 of core member 350 aligns with the central lumen of the core member hub 368.

The core member hub 368 includes a hub shoulder 367 at the distal end extending from the distal end of the core member hub 368 toward a proximal end of the core member hub 368. The hub shoulder 367 has a smaller diameter than the main body portion 365 of the core member hub 368 such that the hub shoulder 367 is sized and configured to be received within a central lumen 384 of the slot sleeve 370. In particular, the hub shoulder 367 is sized and configured to be movably received within the large diameter proximal portion 387 of the slot sleeve 370. In some examples, the main body portion 365 of the core member hub 368 includes a gripping feature 363 to assist the user in manipulating the core member 350. For example, the main body portion 365 can include one or more opposing depressions/recesses that provide a gripping surface.

The core member 350 includes a traveling pin 358 for controlling movement of the core member 350 within the slot sleeve 370 and the introducer body 310. As illustrated in FIGS. 48 and 49, the traveling pin 358 extends radially from the shoulder 367 of the core member hub 368 and into the guide slot 372 of the slot sleeve 370. In some examples, the traveling pin 358 includes a metal pin, for example, stainless steel pin. The traveling pin 358 moves within the guide slot 372 provided on the slot sleeve 370, controlling movement of the core member 350 between the unexpanded and expanded positions.

In some implementations, the introducer system 300 includes a connecting sleeve 390 for coupling the slot sleeve 370 and the core member hub 368 to the sheath hub 20. FIG. 51 provides a side view of the introducer system 300 of FIG. 24 coupled to the sheath hub 20 of sheath 8. The expandable sheath 8 and sheath hub 20 shown in FIG. 51 correspond to other expandable sheath configurations described herein, for example, the layered configuration illustrated in FIGS. 11-14 and/or FIGS. 15-23. As described herein, in some aspects, the sheath 8 includes a continuous inner layer (for example, inner layer 102, 202) defining a central lumen extending through the sheath 8, and a tubular strain relief layer 26 extending along the outer surface of the sheath 8 that limits radial expansion of the portion of the sheath 8 proximate the sheath hub 20. As described herein, the various layers of the sheath 8 and the strain relief layer 26 are configured to locally expand from an unexpanded configuration, at a first diameter, to an expanded configuration at a second, larger, diameter, due to the outwardly directed radial force exerted on the lumen of the sheath (for example, inner layer 202) by the expandable introducer system 300 and/or a medical device, and then locally contract at least partially back to the unexpanded configuration as the dilator and/or medical device passes through the lumen. The introducer system 300 is sized and configured to be received within and expand the lumen of the sheath 8.

As illustrated in FIGS. 52-58, the connecting sleeve 390 includes including a sleeve body 392 having a proximal end 394 and a distal end 396 and defining a central lumen 398 extending longitudinally between the proximal end 394 and the distal end 396. The distal end 396 of the connecting sleeve 390 is received within the central opening of the sheath hub 20. Similar to how the locking sleeve 28 is coupled to the sheath hub 20 (FIG. 6), the connecting sleeve 390 includes interference diameters 395 sized and configured to be received within the receiving slots 48/openings provided in the side wall of the sheath hub 20. The interference diameters 395 extend radially from the outer surface of the slot sleeve body 392 around (all or a portion of) the circumference of the connecting sleeve 390. Coupling between the receiving slots 48 and the interference diameters 395 axially and rotationally fixes the connecting sleeve 390 and the sheath hub 20 relative to each other.

In some aspects, the distal end of the slot sleeve 370 is received within the central lumen 384 of the connecting sleeve 390. As illustrated in FIGS. 51, 53, and 57, the connecting sleeve 390 includes a connecting sleeve guide slot 391 for receiving a coupling pin 376 provided on the slot sleeve 370. The coupling pin 376 extends radially from the outer surface 378 of the slot sleeve 370 as shown in FIG. 26, 37, 51. The coupling pin 376 is received and movable within the connecting sleeve guide slot 391 for directing/controlling movement of the slot sleeve 370 between an unexpanded and an expanded position and for coupling the introducer system 300 to a sheath assembly 10/sheath hub 20. The coupling pin 376 is coupled to the slot sleeve 370 by a spring support such that the coupling pin 376 moves radially (for example, in and out with respect to the longitudinal centerline of the slot sleeve 370) allowing the slot sleeve 370 to be movably coupled to the connecting sleeve 390.

As illustrated in FIGS. 53 and 57, the connecting sleeve guide slot 391 is generally L-shaped. In some examples, the connecting sleeve guide slot 391 includes a circumferentially-extending proximal portion that allows the slot sleeve 370 to rotate with respect to the connecting sleeve 390. The connecting sleeve guide slot 391 further includes an axially-extending distal portion that allows the slot sleeve 370 to move axially with respect to the connecting sleeve 390. Similar to the guide slot 372 provided on slot sleeve 370, the connecting sleeve guide slot 391 can include a catch for maintaining the coupling pin 376 in the desired position within the connecting sleeve guide slot 391.

Example materials and/or compositions for the various components of the introducer system 300 are provided here. In some implementations, the introducer body 310 is formed from a stiffer material than the introducer tip 330. For example, the introducer body 310 is constructed from a stiffer material to allow for manufacturing of the slotted openings 320, which are difficult to manufacture in a soft material. In another implementation, the introducer body 310 is formed from polyethylene including, for example, a material comprising low-density polyethylene (LDPE) 60%, high performance polyethylene (HPPE) 20%, and barium sulfate (BaSO4) 20%). In a further aspect, introducer tip 330 is formed from polyethylene including, for example, a material comprising low-density polyethylene (LDPE) 70%, high performance polyethylene (HPPE) 10%, and barium sulfate (BaSO4) 20%. In another aspect, the core member 350 is formed from a polymeric material including, for example, polyethylene (LDPE) 60%, high performance polyethylene (HPPE) 20%, and barium sulfate (BaSO4) 20%. In a further aspect, the slot sleeve 370 is formed from a polycarbonate including, for example, an overmolded polycarbonate.

A method of pre-dilating/expanding a sheath that can be used to deliver a medical device (for example, to a procedure site) is described herein. In general, and described in more detail herein, the introducer body 310 is inserted into the sheath 8 in an unexpanded configuration. As the core member 350 moves within the central lumen 312 of the introducer body 310, interference between the core member 350 and the central lumen 312 causes the introducer body 310 to radially expand into an expanded configuration. The introducer system 300, in the expanded configuration, can then be advanced and/or withdrawn within the central lumen of sheath 8 so as to expand/dilate the desired length and portions of the sheath 8. With the desired portions of the sheath 8 expanded/pre-dilated, the introducer system 300 is then removed from the sheath 8 (in either the expanded or unexpanded configuration), and a medical device can be inserted at lower push force than required for a non-dilated sheath.

The method includes advancing or otherwise positioning the elongated core member 350 into the central lumen 312 of an introducer body 310 as illustrated in FIGS. 59 and 60. As described herein, at least a portion of the introducer body 310 is configured to expand from an unexpanded configuration (FIGS. 30, 31A, 32, and 60) in which the introducer body 310 has a first outer diameter to an expanded configuration (FIGS. 31B, 33, 61, and 62) in which the introducer body 310 has a second, larger, outer diameter due to an outwardly directed radial force exerted on the central lumen 312 of introducer body 310 by the core member 350 as it moves between an unexpanded and expanded position within the central lumen 312.

FIG. 60 illustrates the core member 350 in the unexpanded (home) position within the central lumen 312. There the expansion element 354 is positioned within the cavity 322. Because the cavity 322 has a size and shape corresponding to the size and shape of the expansion element 354, and a diameter greater than a diameter of a main portion 324 of the central lumen 312, the introducer body 310 is not expanded. As will be described in more detail herein, advancing the core member 350 to the expanded position within the central lumen 312 includes moving the core member 350 such that the expansion element 354 moves from the cavity 322 and into the (smaller diameter) main body portion 328 of the central lumen 312, thereby causing the introducer body 310 to expand (FIGS. 62-63).

The method further includes next coupling the core member 350 to the introducer body 310. For example, the traveling pin 358 extending radially from the core member 350 is engaged within a guide slot 372 provided on a slot sleeve 370 (coupled to a proximal end 314 of the introducer body 310). In some aspects, the traveling pin 358 is radially movable (for example, in and out) with respect an outer surface of the core member 350 (and/or core member hub 368) and includes a spring support biasing the traveling pin 358 in an outward position. As such, coupling the core member 350 to the introducer body 310 includes: depressing the traveling pin 358 such that the core member 350 (and/or core member hub 368) and traveling pin 358 are movable (for example, rotationally and/or axially) within a central lumen 384 of the slot sleeve 370. For example, the traveling pin 358 is depressed to have a height corresponding to the outer surface of the core member hub 368 such that the depressed traveling pin 358 is axially and rotationally movable within the central lumen 384 of the slot sleeve 370. The core member 350 and/or core member hub 368 are then rotationally and/or axially moved until the traveling pin 358 is received within the guide slot 372 and the traveling pin 358 is released from its depressed condition coupling the core member 350 to the introducer body 310.

In some implementations, the core member 350 is secured to the slot sleeve 370 in the unexpanded (home) position by a sleeve spring 380 as shown in FIG. 48 provided between a distal end of the core member hub 368 and an inner shoulder 382 provided on the central lumen 384 of the slot sleeve 370. For example, the slot sleeve spring 380 biases the core member 350 proximally, keeping the expansion element 354 is the home/unexpanded position. The core member 350 is pushed distally, moving the traveling pin 358 within the guide slot 372 toward the expanded position, as shown in FIGS. 31, 61, and 62.

The method further includes positioning/locating the core member 350 in the unexpanded (home) position within the central lumen 312 of the introducer body 310. In some aspects, positioning the core member 350 in the unexpanded (home) position within the central lumen 312 of the introducer body 310 includes positioning the traveling pin 358 at an unexpanded (home) position within the guide slot 372 as shown in FIG. 59.

The method further includes advancing the coupled core member 350 and introducer body 310 into central lumen of an expandable sheath 8 through the sheath hub 20. The method includes providing a sheath 8 according to any of the examples described herein. The sheath 8 includes a continuous inner layer (for example, inner layers 102, 104, 106, 202) defining a central lumen extending therethrough and a tubular strain relief layer 26 provided over the inner layer at a proximal end of the sheath 8 and extending along at least a portion of a length of the sheath 8. As described herein, the strain relief layer 26 provides a region of higher durometer or stiffness that restricts expansion of the underlying sheath layers. In some examples, the sheath 8 includes an outer layer (for example, fourth layer 108, outer layer 204) provided over the inner layer, and under or over the tubular strain relief layer 26. The strain relief layer 26 is provided at the proximal end of the sheath 8 and extends along a least a portion of the length of the sheath 8.

The method further includes coupling the introducer body 310 to the sheath 8. For example, the introducer body 310 can be coupled to the sheath 8 via the slot sleeve 370 and sheath connecting sleeve 390. The sheath connecting sleeve 390 is coupled to the sheath hub 20 by engagement between the interference diameters 395 and the receiving slots 48/openings provided in the side wall of the sheath hub 20 as described herein. The sheath hub 20 is fixedly (for example, axially and rotationally fixed) coupled to the sheath 8. The slot sleeve 370 is coupled to the sheath connecting sleeve 390 by engaging the coupling pin 376 extending radially from the slot sleeve 370 within the connecting sleeve guide slot 391 provided on the sheath connecting sleeve 390, where the sheath connecting sleeve 390 is coupled to a proximal end of the expandable sheath 8, as illustrated in FIG. 63. In some implementations, the coupling pin 376 is radially movable (for example, in and out) with respect the outer surface of the slot sleeve 370 and includes a spring support biasing the coupling pin 376 in an outward position. Coupling the slot sleeve 370 to the sheath connecting sleeve 390 includes depressing the coupling pin 376 such that the slot sleeve 370 and coupling pin 376 arc movable (for example, rotationally and axially) within a central lumen of the sheath connecting sleeve 390. For example, depressing the coupling pin 376 to have a height corresponding to the outer surface of the slot sleeve 370 such that the depressed coupling pin 376 is axially and rotationally movable withing the central lumen 398 of the sheath connecting sleeve 390. The slot sleeve 370 is then moved rotationally and/or axially until the coupling pin 376 is received within the connecting sleeve guide slot 391 and the coupling pin 376 is released from its depressed condition.

The method further includes positioning/locating the introducer body 310 (for example, coupled introducer body 310 and slot sleeve 370) in an unexpanded (home) position with respect to the sheath 8. In some aspects, positioning the introducer body 310 (for example, coupled introducer body 310 and slot sleeve 370) in an unexpanded (home) position with respect to the sheath 8 (for example, with respect to the sheath connecting sleeve 390) includes positioning/moving the coupling pin 376 at an unexpanded (home) position within the connecting sleeve guide slot 391 as shown in FIGS. 59 and 63. In some aspects, the slot sleeve 370 is secured in the unexpanded (home) position by engagement between the coupling pin 376 and the connecting sleeve guide slot 391. For example, the coupling pin 376 is retained in the proximal most portion connecting sleeve guide slot 391, for example, the circumferentially-extending proximal portion of the connecting sleeve guide slot 391. In some examples, the connecting sleeve guide slot 391 includes a catch for maintaining the coupling pin 376 in the unexpanded (home) position.

The method further includes advancing the core member 350 within the central lumen 312 of the introducer body 310 such that the core member 350 moves from the unexpanded (home) position to the expanded position (FIGS. 61 and 62) where the core member 350 exerts an outwardly directed radial force on the central lumen 312 expanding the introducer body 310 and a corresponding portion of the sheath 8.

In some aspects, advancing the core member 350 to the expanded position includes advancing the core member 350 distally within the introducer body 310.

For example, advancing the core member 350 to the expanded position includes advancing the traveling pin 358 (extending radially from the core member 350) within a guide slot 372 provided on the slot sleeve 370 (coupled to the proximal end 314 of the introducer body 310) to/toward a traveling pin expanded position shown in FIG. 63. The traveling pin 358 is advanced distally along the axially-extending proximal portion of the guide slot 372 and rotationally within the circumferentially-extending distal portion of the guide slot 372. As illustrated in FIGS. 62 and 63, when the core member 350 is in the expanded position, contact between the expansion element 354 and the central lumen 312 of the introducer body 310 provides the outwardly directed radial force on the central lumen 312 of the introducer body 310 directing expansion of the introducer body 310.

In the expanded configuration, the introducer body 310 exerts an outwardly directed radial force against the central lumen (for example, inner layer) of the sheath 8. This causes the sheath 8, including the inner layer and/or strain relief layer 26, proximate the expanded portion of the introducer body 310/expansion element 354 to locally expand from an unexpanded configuration to an expanded configuration. The sheath 8, including the inner layer and/or strain relief layer 26, is locally contracted towards the unexpanded configuration as the expansion element 354/expanded portion of the introducer body 310 passes through the corresponding portion of the central lumen of sheath 8.

The method further includes expanding a distal opening of the sheath 8 as shown in FIGS. 64-69. The distal opening of the sheath 8 is expanded by positioning the core member 350 in the expanded position and advancing the core member 350 and the introducer body 310 within the central lumen of the sheath 8 such that an expanded portion of the introducer body 310 is advanced through a distal opening of the sheath 8. As a result, the core member 350/introducer body 310 exert an outwardly directed radial force expanding the distal opening of the sheath 8, as illustrated in FIGS. 64 and 65.

In some implementations, the core member 350 and introducer body 310 are coupled together (for example, axially and/or rotationally fixed) before the combined core member 350 and introducer body 310 is advanced through the distal opening of the sheath 8. For example, the core member 350 to the introducer body 310 are coupled by engaging a traveling pin 358 extending radially from the core member 350 within the guide slot 372 provided on a slot sleeve 370 (coupled to a proximal end 314 of the introducer body 310). The introducer body 310 is then coupled to the sheath 8 by coupling the slot sleeve 370 to a sheath connecting sleeve 390. For example, the coupling pin 376 extending radially from the slot sleeve 370 is engaged within a connecting sleeve guide slot 391 provided on the sheath connecting sleeve 390 (where the sheath connecting sleeve 390 is coupled to a proximal end of the expandable sheath 8). The core member 350 is then advanced to the expanded position by advancing the traveling pin 358 within the guide slot 372 to a traveling pin expanded position shown in FIG. 63. For example, the traveling pin 358 is advanced distally along the axially-extending proximal portion of the guide slot 372 and rotationally within the circumferentially-extending distal portion of the guide slot 372. The slot sleeve 370 is then advanced within the connecting sleeve 390 to the expanded position (for example, distal tip expanded configuration) by advancing the coupling pin 376 with the connecting sleeve guide slot 391 to a coupling pin expanded position as shown in FIG. 66. For example, by advancing the coupling pin 376 rotationally within the circumferentially-extending proximal portion of the connecting sleeve guide slot 391 and distally along the axially-extending distal portion of the connecting sleeve guide slot 391. As a result, the combined introducer body 310 and core member 350 (in the expanded position) are moved through the distal opening of the sheath 8 as shown in FIGS. 67, 68, and 69.

In some implementations, the sheath 8 is expanded during withdrawal of the introducer system 300/expanded introducer body 310 through the sheath 8. As shown in FIGS. 70 and 71, the slot sleeve 370 is withdrawn from the connecting sleeve 390 by withdrawing (proximally) the coupling pin 376 within the connecting sleeve guide slot 391 to a coupling pin 376 unexpanded (home) position. For example, by withdrawing the coupling pin 376 proximally along the axially-extending distal portion of the connecting sleeve guide slot 391 and rotationally within the circumferentially-extending proximal portion of the connecting sleeve guide slot 391. As a result, the combined introducer body 310 and core member 350 (in the expanded position) are moved proximally back through the distal opening of the sheath 8. In some aspects, the coupling pin 376 is depressed such that the slot sleeve 370 and the coupling pin 376 are movable (for example, rotationally and/or axially) within the central lumen 398 of the connecting sleeve 390. For example, the coupling pin 376 is depressed to have a height corresponding to the outer surface of the slot sleeve 370 such that the depressed coupling pin 376 is axially and rotationally movable withing the central lumen 398 of the connecting sleeve 390. The slot sleeve 370 can now be removed from the connecting sleeve 390 and the introducer body 310 withdrawn from the central lumen of the sheath 8 as shown in FIG. 71.

When used to deliver a medical device to a treatment site within a patient, the sheath 8 is inserted at least partially into the blood vessel of the patient and the distal end of the sheath 8 is positioned at a location proximate the treatment site. The pre-dilating step of expanding the sheath 8 can be completed in advance of inserting the sheath 8 into the patient and/or after the sheath 8 is inserted into the patient.

A medical device is then introduced into the central lumen of the (pre-dilated) sheath 8 with greater case.

The medical device is advanced through a portion of the sheath 8 corresponding to the strain relief layer 26 and exerts an outwardly directed radial force against the central lumen of the sheath 8 (for example, inner layer) and causing the sheath 8 (including the inner layer and/or the strain relief layer 26) proximate the medical device to locally expand from an unexpanded configuration (FIGS. 17-19 and 22) to an expanded configuration (FIGS. 20 and 23). In some examples, the medical device is contracted or compressed radially as it passes through the strain relief layer 26, from the proximal portion 242, through the tapered portion 248 and into the smaller diameter distal portion 246.

As the medical device passes through the corresponding portion of the lumen of sheath 8, the sheath 8 and strain relief layer 26 locally expand towards/to the expanded configuration.

The medical device is then advanced beyond the distal end 336 of the strain relief layer 26 and into the lumen of the longitudinally body portion of the sheath 8 (beyond the strain relief layer 26) and beyond the distal opening. As the medical device is advanced through the sheath 8 beyond the strain relief layer 26, and through the distal opening, the sheath 8 locally expands from the unexpanded configuration (FIGS. 11-13A, and 17-19) to the expanded configuration (FIGS. 14 and 20) at a location proximate the medical device in response to the outwardly directed radial force of the medical device exerted against the inner layer/central lumen of the sheath 8.

As the medical device passes through the lumen of the sheath 8, the sheath 8 locally contracts at least partially back to the unexpanded configuration (FIGS. 11-13A and 17-19). When used to deliver a medical device to a treatment site within a patient, the medical device is then passed through the distal tip/distal opening of the sheath 8 and delivered to the treatment site. The position of the medical device can be moved or adjusted until the medical device is adequately positioned within the patient. With the medical device delivered to the treatment site, any delivery system/components coupled to the medical device are then removed from the medical device and withdrawn from the lumen of the sheath 8. The sheath 8 is removed from the patient and the opening in the blood vessel and skin closed.

In some examples, at least one of the inner layer and/or outer layer includes at least one folded portion, for example, ridges 126 and valleys 128 of the fourth (outer) layer 108 of the sheath 8 illustrated in FIGS. 11-14, and folded portion 218 of the inner layer 202 of the sheath 8 illustrated in FIGS. 15-23. Locally expanding the lumen of the sheath 8 causes a corresponding length of the folded portion to at least partially unfold. Similarly, locally contracting the sheath 8 at least partially back to the unexpanded configuration causes a length of the folded portion to urge back towards a folded configuration.

In some examples, the outer layer is a discontinuous outer layer and includes an overlapping portion (for example, overlapping portion 22) and an underlying portion (for example, underlying portion 222). When the sheath 8 is in the unexpanded configuration, the overlapping portion overlaps the underlying portion with the folded portion of the inner layer disposed between the overlapping portion and the underlying portion (FIGS. 17, 19, 22, and 23). As the sheath 8 locally expands to/toward the expanded configuration, a length of the overlapping portion moves circumferentially with respect to the underlying portion unfolding. As illustrated in FIG. 20, when the sheath 8 is fully expanded, the inner layer extends into the gap 232 formed between the longitudinal edges of the overlapping portion 220 and the underlying portion 222 of the outer layer 204.

In some examples, the sheath 8 includes an elastic outer layer 250 that extends at least partially over the outer layer and/or the strain relief layer 26. The elastic outer layer 250 locally expands and contracts as the medical device is advanced through the lumen of the sheath 8. In some examples, the elastic outer layer 250 urges the various layers of the sheath 8 toward an unexpanded configuration.

The medical device described herein can include a prosthetic device mounted in a radially crimped state on a delivery apparatus, and the act of advancing the prosthetic device through the lumen of the sheath 8 comprises advancing the delivery apparatus and the prosthetic device through lumen of the sheath 8 and into the vasculature of the patient. In some examples, the prosthetic device comprises a prosthetic heart valve and the method further comprises implanting the prosthetic heart valve at a treatment site within the patient. As described herein, the prosthetic heart valve is mounted on a balloon catheter of the delivery apparatus as the prosthetic heart valve is advanced through the sheath 8.

### Exemplary Aspects

In view of the many possible aspects to which the principles of the disclosed disclosure can be applied, it should be recognized that the illustrated aspects are only preferred examples of the disclosure and should not be taken as limiting the scope of the disclosure. Rather, the scope of the disclosure is defined by the following claims. We, therefore, claim as our disclosure all that comes within the scope and spirit of these claims.
Example 1: An expandable introducer assembly including: an elongated introducer body including: a central lumen extends between a proximal and distal end of the introducer body; a slit extending axially from a distal end toward a proximal end of the introducer body; and a slotted opening extending axially along a portion of the introducer body; a distal introducer tip coupled to the distal end of the of the introducer body; an elongated core member received within the central lumen of the introducer body and movable between an unexpanded and an expanded position, wherein at least a portion of the introducer body is configured to expand from an unexpanded configuration in which the introducer body has a first outer diameter to an expanded configuration in which the introducer body has a second, larger, outer diameter due to an outwardly directed radial force exerted on the central lumen of introducer body by the core member as it moves between the unexpanded and expanded positions.
Example 2: The introducer assembly according to any example herein, particularly example 1, wherein the core member includes an elongated body portion and an expansion element extending radially from an outer surface of the body portion, wherein contact between the expansion element and the central lumen of the introducer body provides the outwardly directed radial force on the central lumen of the introducer body for directing expansion of the introducer body.
Example 3: The introducer assembly according to any example herein, particularly example 2, wherein the central lumen of the introducer body includes a cavity for receiving the expansion element when the core member is in the unexpanded position, the cavity having a size and shape corresponding to the size and shape of the expansion element, the cavity having a diameter greater than a diameter of a main portion of the central lumen, wherein, when the core member is in the unexpanded position, the expansion element is received within the cavity and the introducer body is not expanded, wherein, when the core member is moved to the expanded position, the core member is moved axially and the expansion element is moved from the cavity to the main portion of the central lumen, thereby causing the introducer body to expand.
Example 4: The introducer assembly according to any example herein, particularly examples 1-3, wherein a portion of the introducer body including the slotted opening defines a portion of the introducer body that is movable between the unexpanded and the expanded configuration, wherein the cavity is provided within the central lumen at a location corresponding to the slotted opening.
Example 5: The introducer assembly according to any example herein, particularly examples 1-4, wherein the introducer body includes a plurality of slits extending from the distal end toward the proximal end of the introducer body.
Example 6: The introducer assembly according to any example herein, particularly example 5, wherein the plurality of slits are evenly spaced around the circumference of the introducer body.
Example 7: The introducer assembly according to any example herein, particularly examples 1-6, wherein the slit extends at least partially through a wall thickness of the introducer body.
Example 8: The introducer assembly according to any example herein, particularly examples 1-7, wherein the slit extends through the entire wall thickness of the introducer body.
Example 9: The introducer assembly according to any example herein, particularly examples 1-8, wherein the slit is provided on a shoulder extending from the distal end of the introducer body, the shoulder having a smaller diameter than a main body portion of the introducer body.
Example 10: The introducer assembly according to any example herein, particularly examples 1-9, wherein a width of the slit is constant along an entire length of the slit.
Example 11: The introducer assembly according to any example herein, particularly examples 1-10, wherein the introducer body includes a plurality of slotted openings.
Example 12: The introducer assembly according to any example herein, particularly example 11, wherein the plurality of slotted openings are evenly spaced around the circumference of the introducer body.
Example 13: The introducer assembly according to any example herein, particularly examples 11-13, wherein the introducer body includes ten slotted openings.
Example 14: The introducer assembly according to any example herein, particularly examples 1-13, wherein the slotted opening extends at least partially through an entire wall thickness of the introducer body.
Example 15: The introducer assembly according to any example herein, particularly examples 1-14, wherein the slotted opening extends through the entire wall thickness of the introducer body.
Example 16: The introducer assembly according to any example herein, particularly examples 1-15, wherein the slotted opening extends along a portion of the introducer body between the proximal and distal ends.
Example 17: The introducer assembly according to any example herein, particularly examples 1-16, wherein the introducer body further includes: a shoulder extending from the distal end of the introducer body toward a proximal end of the body, the shoulder having a smaller diameter than a main body portion of the introducer body, where the shoulder is sized and configured to be received within a central lumen of the distal introducer tip, wherein the slotted opening is provided on the main body portion of the introducer body.
Example 18: The introducer assembly according to any example herein, particularly example 17, wherein a portion of the slotted opening extends along a portion of the shoulder.
Example 19: The introducer assembly according to any example herein, particularly example 18, wherein the slit is provided on the shoulder extending from the distal end of the introducer body, wherein a portion of a length of the slit axially overlaps with a portion of a length of the slotted opening along the shoulder.
Example 20: The introducer assembly according to any example herein, particularly examples 1-19, wherein a width of the slotted opening tapers at a distal end of the slotted opening.
Example 21: The introducer assembly according to any example herein, particularly examples 1-20, wherein the introducer body further includes: a shoulder extending from the distal end of the introducer body toward a proximal end of the introducer body, the shoulder having a smaller diameter than a main body portion of the introducer body, where the shoulder is sized and configured to be received within a central lumen of the introducer tip.
Example 22: The introducer assembly according to any example herein, particularly example 21, wherein the shoulder extends around a circumference of the introducer body and includes a body engagement feature extending circumferentially around the shoulder, wherein the body engagement feature is sized and configured to receive a corresponding tip engagement feature provided on the central lumen of the introducer tip.
Example 23: The introducer assembly according to any example herein, particularly example 22, wherein the body engagement feature includes an annular recess extending circumferentially around the shoulder and the tip engagement feature includes a correspondingly shaped projection extending radially from the central lumen of the distal introducer tip.
Example 24: The introducer assembly according to any example herein, particularly examples 1-23, wherein the introducer tip includes a tapered distal end portion.
Example 25: The introducer assembly according to any example herein, particularly example 24, wherein the tapered distal end portion tapers from a distal end of the introducer tip to a main body portion of the introducer tip (for example, the main body portion including a constant diameter portion extending from the tapered portion toward the proximal end, the tapered distal portion and the main body portion provide a smooth and tight fit between the introducer and the distal opening of the sheath).
Example 26: The introducer assembly according to any example herein, particularly examples 1-25, wherein the introducer tip includes a slit extending axially from a proximal end toward the distal end of the introducer tip.
Example 27: The introducer assembly according to any example herein, particularly examples 1-26, wherein the introducer tip includes a plurality of slits.
Example 28: The introducer assembly according to any example herein, particularly example 27, wherein the plurality of slits are evenly spaced around the circumference of the introducer tip.
Example 29: The introducer assembly according to any example herein, particularly examples 1-28, wherein the slit extends at least partially through a wall thickness of the introducer tip.
Example 30: The introducer assembly according to any example herein, particularly examples 1-29, wherein the slit extends through the entire wall thickness of the introducer tip.
Example 31: The introducer assembly according to any example herein, particularly examples 1-30, wherein the introducer tip includes an annular groove, wherein the introducer body is received within the central lumen of the introducer tip, wherein the introducer tip is coupled to the introducer body by a retaining ring provided over the outer surface of the introducer tip within the annular groove such that radially inward force provided by the retaining ring, compresses the introducer tip between the retaining ring and the introducer body.
Example 32: The introducer assembly according to any example herein, particularly examples 1-31, further including a slot sleeve coupled to the proximal end of the introducer body, the slot sleeve including a guide slot for receiving a traveling pin provided on the core member, the traveling pin movable within the guide slot as the core member moves between an unexpanded and an expanded position.
Example 33: The introducer assembly according to any example herein, particularly examples 1-32, wherein the slot sleeve is fixedly coupled to the proximal end of the introducer body.
Example 34: The introducer assembly according to any example herein, particularly examples 1-33, wherein the guide slot is generally L-shaped, including an axially-extending proximal portion and a circumferentially-extending distal portion.
Example 35: The introducer assembly according to any example herein, particularly examples 1-34, wherein the guide slot includes a catch for maintaining the traveling pin in the expanded position.
Example 36: The introducer assembly according to any example herein, particularly examples 1-35, wherein the expansion element includes a regular or irregular shaped projection extending from the outer surface of the body portion.
Example 37: The introducer assembly according to any example herein, particularly example 36, wherein the expansion element has a circular shape in cross-section. For example, the expansion element can have a generally spherical shape.
Example 38: The introducer assembly according to any example herein, particularly examples 1-37, wherein a diameter of the expansion element is 22 Fr.
Example 39: The introducer assembly according to any example herein, particularly examples 1-38, wherein the core member is received and movable within at least a portion of a central lumen of the introducer tip.
Example 40: The introducer assembly according to any example herein, particularly examples 1-39, wherein the core member includes a central lumen extending therethrough.
Example 41: The introducer assembly according to any example herein, particularly examples 1-40, wherein the core member includes a tapered distal end 362.
Example 42: The introducer assembly according to any example herein, particularly examples 1-41, wherein a proximal end of the core member is coupled to a core member hub.
Example 43: The introducer assembly according to any example herein, particularly example 42, wherein the traveling pin is provided on the core member hub, the traveling pin extending radially from the core member hub.
Example 44: The introducer assembly according to any example herein, particularly examples 42-43, wherein the core member hub includes a hub shoulder 367 extending from a distal end of the core member hub toward a proximal end of the core member hub, the hub shoulder 367 having a smaller diameter than a main body portion 365 of the core member hub, wherein the hub shoulder 367 is sized and configured to be received within a central lumen of the slot sleeve.
Example 45: The introducer assembly according to any example herein, particularly examples 42-44, wherein the core member hub includes a gripping feature 363 including one or more opposing depressions/recesses.
Example 46: The introducer assembly according to any example herein, particularly examples 1-45, further including: a connecting sleeve including a sleeve body having a proximal end and a distal end and defining a central lumen extending longitudinally between the proximal end and the distal end, wherein the distal end of the slot sleeve is received within the central lumen of the connecting sleeve, wherein the connecting sleeve includes a connecting sleeve guide slot for receiving a coupling pin provided on the slot sleeve, the coupling pin extending radially from an outer surface of the slot sleeve, coupling pin received and movable within the connecting sleeve guide slot for moving the slot sleeve between an unexpanded and an expanded position and for coupling the introducer assembly to a sheath assembly.
Example 47: The introducer assembly according to any example herein, particularly example 46, wherein the coupling pin is coupled to the slot sleeve by a spring support such that the coupling pin moves radially allowing the slot sleeve to couple to the connecting sleeve.
Example 48: The introducer assembly according to any example herein, particularly examples 46-47, wherein the connecting sleeve guide slot is generally L-shaped, including a circumferentially-extending proximal portion and an axially-extending distal portion.
Example 49: The introducer assembly according to any example herein, particularly examples 46-48, wherein the connecting sleeve is coupled to a sheath hub, the sheath hub having an elongated body portion with a central lumen extending therethrough and an expandable sheath coupled to a distal end of the body portion, where a central lumen of the expandable sheath is aligned with the central lumens of the sheath hub and the connecting sleeve.
Example 50: The introducer assembly according to any example herein, particularly example 49, wherein the expandable sheath includes a continuous inner layer defining a lumen therethrough, the inner layer having at least one folded portion.
Example 51: The introducer assembly according to any example herein, particularly examples 1-50, wherein the introducer body is formed form a stiffer material than the introducer tip (for example, the introducer body is constructed from a stiffer material to allow for manufacturing of the slotted openings, which are too difficult to manufacture in a soft material).
Example 52: The introducer assembly according to any example herein, particularly examples 1-51, wherein the introducer body is formed from polyethylene including a material comprising low-density polyethylene (LDPE) 60%, high performance polyethylene (HPPE) 20%, and barium sulfate (BaSO4) 20%.
Example 53: The introducer assembly according to any example herein, particularly examples 1-52, wherein the introducer tip is formed from polyethylene including a material comprising low-density polyethylene (LDPE) 70%, high performance polyethylene (HPPE) 10%, and barium sulfate (BaSO4) 20%.
Example 54: The introducer assembly according to any example herein, particularly examples 1-53, wherein the core member is formed from a polymeric material including polyethylene (LDPE) 60%, high performance polyethylene (HPPE) 20%, and barium sulfate (BaSO4) 20%.
Example 55: The introducer assembly according to any example herein, particularly examples 1-54, wherein the slot sleeve is formed from a polycarbonate.
Example 56: A method of expanding a sheath according to any example herein, the method comprising: advancing the elongated core member into the central lumen of an introducer body, at least a portion of the introducer body is configured to expand from an unexpanded configuration in which the introducer body has a first outer diameter to an expanded configuration in which the introducer body has a second, larger, outer diameter due to an outwardly directed radial force exerted on the central lumen of introducer body by the core member as it moves between an unexpanded and expanded position within the central lumen; coupling the core member to the introducer body; positioning the core member in the unexpanded (home) position within the central lumen of the introducer body; advancing the coupled core member and introducer body into central lumen of an expandable sheath; coupling the introducer body to the sheath; positioning the introducer body in an unexpanded (home) position with respect to the sheath; and advancing the core member within the central lumen of the introducer body such that the core member moves from the unexpanded (home) position to the expanded position where the core member exerts an outwardly directed radial force on the central lumen expanding the introducer body and a corresponding portion of the sheath.
Example 57: The method according to any example herein, particularly example 56, wherein advancing the core member to the expanded position includes advancing the core member distally within the introducer body.
Example 58: The method according to any example herein, particularly examples 56-57, wherein advancing the core member to the expanded position includes advancing a traveling pin extending radially from the core member within a guide slot, provided on a slot sleeve coupled to a proximal end of the introducer body, to a traveling pin expanded position.
Example 59: The method according to any example herein, particularly examples 56-58, wherein the core member includes an elongated body portion and an expansion element extending radially from an outer surface of the body portion, wherein, when the core member is in the expanded position, contact between the expansion element and the central lumen of the introducer body provides the outwardly directed radial force on the central lumen of the introducer body directing expansion of the introducer body.
Example 60: The method according to any example herein, particularly example 59, wherein the central lumen of the introducer body includes a cavity for receiving the expansion element when the core member is in the unexpanded position, the cavity having a size and shape corresponding to the size and shape of the expansion element, the cavity having a diameter greater than a diameter of a main portion of the central lumen, wherein positioning the core member in the unexpanded (home) position within the central lumen of the introducer body includes positioning expansion element within the cavity such that the introducer body is not expanded, wherein advancing the core member to the expanded position within the central lumen includes moving the core member such that the expansion element moves from the cavity and into the main portion of the central lumen causing the introducer body to expand.
Example 61: The method according to any example herein, particularly examples 56-60, further including: expanding a distal opening of the sheath by positioning the core member in the expanded position and advancing the core member and the introducer body within the central lumen of the sheath such that an expanded portion of the introducer body is advanced through a distal opening of the sheath, where the core member/introducer body exert an outwardly directed radial force expanding the distal opening.
Example 62: The method according to any example herein, particularly example 61, wherein the core member and introducer body are coupled together before the combined core member and introducer body is advanced through the distal opening of the sheath.
Example 63: The method according to any example herein, particularly examples 61-62, wherein expanding the distal opening further includes: coupling the core member to the introducer body by engaging a traveling pin extending radially from the core member within a guide slot provided on a slot sleeve coupled to a proximal end of the introducer body; coupling the introducer body to the sheath by coupling the slot sleeve to a sheath connecting sleeve, engaging a coupling pin extending radially from the slot sleeve within a connecting sleeve guide slot provided on the sheath connecting sleeve, where the sheath connecting sleeve is coupled to a proximal end of the expandable sheath; advancing the core member to the expanded position by advancing the traveling pin within the guide slot to a traveling pin expanded position; and advancing the slot sleeve within the connecting sleeve to the expanded position by advancing the coupling pin with the connecting sleeve guide slot to a coupling pin expanded position, thereby moving the combined introducer body and core member through the distal opening of the sheath.
Example 64: The method according to any example herein, particularly example 63, further including: withdrawing the expanded introducer body through the sheath thereby expanding the sheath by: withdrawing the slot sleeve from the connecting sleeve by withdrawing the coupling pin with in the connecting sleeve guide slot to a coupling pin unexpanded (home) position, thereby moving the combined introducer body and core member; depressing the coupling pin such that the slot sleeve and the coupling pin are movable within the central lumen of the connecting sleeve; removing the slot sleeve from the connecting sleeve; and withdrawing the introducer body from the central lumen of the sheath.
Example 65: The method according to any example herein, particularly examples 56-64, wherein coupling the core member to the introducer body includes: engaging a traveling pin extending radially from the core member within a guide slot provided on a slot sleeve coupled to a proximal end of the introducer body.
Example 66: The method according to any example herein, particularly example 65, wherein the traveling pin of the core member is radially movable with respect an outer surface of the core member and includes a spring support biasing the traveling pin in an outward position, wherein coupling the core member to the introducer body includes: depressing the traveling pin such that the core member (and/or core member hub) and traveling pin are movable within a central lumen of the slot sleeve; and moving the core member and/or core member hub rotationally and/or axially until the traveling pin is received within the guide slot and the traveling pin is released from its depressed condition.
Example 67: The method according to any example herein, particularly examples 56-66, wherein the core member is secured to the slot sleeve in the unexpanded (home) position by sleeve spring provided between a distal end of the core member hub and an inner shoulder provided on the central lumen of the slot sleeve.
Example 68: The method according to any example herein, particularly examples 56-67, wherein positioning the core member in the unexpanded (home) position within the central lumen of the introducer body includes: positioning the traveling pin located at an unexpanded (home) position within the guide slot.
Example 69: The method according to any example herein, particularly examples 56-68, wherein positioning the introducer body in an unexpanded (home) position with respect to the sheath includes: positioning the coupling pin is located at an unexpanded (home) position within the connecting sleeve guide slot.
Example 70: The method according to any example herein, particularly examples 56-69, wherein coupling the introducer body to the sheath includes: coupling the introducer body to the sheath via a slot sleeve coupled to a proximal end of the introducer body.
Example 70: The method according to any example herein, particularly example 70, wherein coupling the introducer body to the sheath includes coupling the slot sleeve to a sheath connecting sleeve, by engaging a coupling pin extending radially from the slot sleeve within a connecting sleeve guide slot provided on the sheath connecting sleeve, where the sheath connecting sleeve is coupled to a proximal end of the expandable sheath.
Example 71: The method according to any example herein, particularly examples 56-71, wherein the coupling pin is radially movable with respect the outer surface of the slot sleeve and includes a spring support biasing the coupling pin in an outward position, wherein coupling the slot sleeve to the sheath connecting sleeve includes: depressing the coupling pin such that the slot sleeve and coupling pin are movable within a central lumen of the sheath connecting sleeve; and moving the slot sleeve rotationally and/or axially until the coupling pin is received within the connecting sleeve guide slot and the coupling pin is released from its depressed condition.
Example 72: The method according to any example herein, particularly examples 56-72, wherein the slot sleeve is secured in the unexpanded (home) position by engagement between the coupling pin and the connecting sleeve guide slot, in some examples, the connecting sleeve guide slot includes a catch for maintaining the coupling pin in the unexpanded (home) position.
Example 73: The method according to any example herein, particularly examples 56-73, further including: coupling the connecting sleeve to a sheath hub, where the sheath hub is fixedly coupled to the sheath.
Example 75: A method of delivering a medical device through a sheath, the method comprising: providing a radially expandable sheath including a continuous inner layer defining a central lumen therethrough, the inner layer having at least one folded portion extending along a length of the inner layer; advancing the elongated core member into the central lumen of an introducer body, at least a portion of the introducer body is configured to expand from an unexpanded configuration in which the introducer body has a first outer diameter to an expanded configuration in which the introducer body has a second, larger, outer diameter due to an outwardly directed radial force exerted on the central lumen of introducer body by the core member as it moves between an unexpanded and expanded position within the central lumen; coupling the core member to the introducer body; positioning the core member in the unexpanded (home) position within the central lumen of the introducer body; advancing the coupled core member and introducer body into central lumen of the sheath expandable sheath; coupling the introducer body to the sheath; positioning the introducer body in an unexpanded position with respect to the sheath; inserting the introduce body and the sheath at least partially into the blood vessel of the patient; advancing the core member within the central lumen of the introducer body such that the core member moves from the unexpanded (home) position to the expanded position where the core member exerts an outwardly directed radial force on the central lumen expanding the introducer body and a corresponding portion of the sheath; moving the introducer body within the central lumen of the sheath causing the sheath to locally expand from the unexpanded configuration to the expanded configuration at a location proximate the medical device in response to the outwardly directed radial force of the introducer body exerted against the inner layer and locally contracting the sheath at least partially back to the unexpanded configuration as the introducer body passes through the lumen; withdrawing the introducer body from the sheath; advancing a medical device through the central lumen of the sheath and beyond the distal opening of the sheath to the treatment site.
Example 76: The method according to any example herein, particularly example 75, wherein the inner layer includes at least one folded portion, wherein locally expanding the lumen of the sheath causes a length of the folded portion to at least partially unfold.
Example 77: The method according to any example herein, particularly examples 75-76, wherein the sheath further includes: an outer layer provided over the inner layer, where the outer layer is discontinuous and includes an overlapping portion and an underlying portion, wherein when the sheath is in the unexpanded configuration, the overlapping portion overlaps the underlying portion with the folded portion of the inner layer disposed between the overlapping portion and the underlying portion, wherein the strain relief layer 26 extends at least partially over the outer layer.
Example 78: The method according to any example herein, particularly examples 75-77, wherein the medical device is a prosthetic device mounted in a radially crimped state on a delivery apparatus, wherein the prosthetic device comprises a prosthetic heart valve and the method further comprises implanting the prosthetic heart valve at a treatment site within the patient, wherein the prosthetic heart valve is mounted on a balloon catheter of a delivery apparatus as the prosthetic heart valve is advanced through the sheath.
Example 79: The method according to any example herein, particularly examples 75-78, wherein the sheath is inserted into a femoral artery of the patient.

In view of the many possible aspects to which the principles of the disclosed disclosure can be applied, it should be recognized that the illustrated aspects are only preferred examples of the disclosure and should not be taken as limiting the scope of the disclosure. Rather, the scope of the disclosure is defined by the following claims. We, therefore, claim as our disclosure all that comes within the scope and spirit of these claims.

What is claimed is:

1. An expandable introducer assembly including:
an elongated introducer body including:
a central lumen extends between a proximal and a distal end of the introducer body;
a slit extending axially from a distal end toward a proximal end of the introducer body; and
a slotted opening extending axially along a portion of the introducer body;
a distal introducer tip coupled to the distal end of the of the introducer body; and
an elongated core member received within the central lumen of the introducer body and movable between an unexpanded and an expanded position,
wherein at least a portion of the introducer body is configured to expand from an unexpanded configuration in which the introducer body has a first outer diameter to an expanded configuration in which the introducer body has a second, larger, outer diameter due to an outwardly directed radial force exerted on the central lumen of introducer body by the core member as it moves between the unexpanded and expanded positions.

2. The introducer assembly of claim 1, wherein the core member includes an elongated body portion and an expansion element extending radially from an outer surface of the body portion,
wherein contact between the expansion element and the central lumen of the introducer body provides the outwardly directed radial force on the central lumen of the introducer body for directing expansion of the introducer body.

3. The introducer assembly of claim 2, wherein the central lumen of the introducer body includes a cavity for receiving the expansion element when the core member is in the unexpanded position, the cavity having a size and shape corresponding to the size and shape of the expansion element, the cavity having a diameter greater than a diameter of a main portion of the central lumen,
wherein, when the core member is in the unexpanded position, the expansion element is received within the cavity and the introducer body is not expanded,
wherein, when the core member is moved to the expanded position, the core member is moved axially and the expansion element is moved from the cavity to the main portion of the central lumen, thereby causing the introducer body to expand.

4. The introducer assembly of claim 3, wherein a portion of the introducer body including the slotted opening defines a portion of the introducer body that is movable between the unexpanded and the expanded configuration,
wherein the cavity is provided within the central lumen at a location corresponding to the slotted opening.

5. The introducer assembly of claim 1, wherein the introducer body includes a plurality of slits extending from the distal end toward the proximal end of the introducer body.

6. The introducer assembly of claim 1, wherein the introducer body includes a plurality of slotted openings extending along a portion of the introducer body between the proximal and distal ends.

7. The introducer assembly of claim 6, wherein the plurality of slotted openings are evenly spaced around a circumference of the introducer body.

8. The introducer assembly of claim 1, wherein the introducer body further includes:
a shoulder extending from the distal end of the introducer body toward a proximal end of the body, the shoulder having a smaller diameter than a main body portion of the introducer body, where the shoulder is sized and configured to be received within a central lumen of the distal introducer tip,
wherein the slotted opening is provided on the main body portion of the introducer body.

9. The introducer assembly of claim 8, wherein the slit is provided on the shoulder extending from the distal end of the introducer body,
wherein a portion of a length of the slit axially overlaps with a portion of a length of the slotted opening along the shoulder.

10. The introducer assembly of claim 1, wherein the shoulder extends around a circumference of the introducer body and includes a body engagement feature extending circumferentially around the shoulder,
wherein the body engagement feature is sized and configured to receive a corresponding tip engagement feature provided on the central lumen of the introducer tip,
wherein the body engagement feature includes an annular recess extending circumferentially around the shoulder and the tip engagement feature includes a correspondingly shaped projection extending radially from the central lumen of the distal introducer tip.

11. The introducer assembly of claim 1, wherein the introducer tip includes a slit extending axially from a proximal end toward the distal end of the introducer tip.

12. The introducer assembly of claim 1, wherein the introducer tip includes an annular groove,
wherein the introducer body is received within the central lumen of the introducer tip,
wherein the introducer tip is coupled to the introducer body by a retaining ring provided over the outer surface of the introducer tip within the annular groove such that radially inward force provided by the retaining ring, compresses the introducer tip between the retaining ring and the introducer body.

13. The introducer assembly of claim 1, further including a slot sleeve coupled to the proximal end of the introducer body, the slot sleeve including a guide slot for receiving a traveling pin provided on the core member, the traveling pin movable within the guide slot as the core member moves between an unexpanded and an expanded position,
wherein the slot sleeve is fixedly coupled to the proximal end of the introducer body,
wherein the guide slot is generally L-shaped, including an axially-extending proximal portion and a circumferentially-extending distal portion.

14. The introducer assembly of claim 1, wherein the core member is received and movable within at least a portion of a central lumen of the introducer tip,
wherein the core member includes a central lumen extending therethrough,
wherein a proximal end of the core member is coupled to a core member hub 368.

15. The introducer assembly of claim 1, further including:
a connecting sleeve including a sleeve body having a proximal end and a distal end and defining a central lumen extending longitudinally between the proximal end and the distal end,
wherein the distal end of the slot sleeve is received within the central lumen of the connecting sleeve,
wherein the connecting sleeve includes a connecting sleeve guide slot for receiving a coupling pin provided on the slot sleeve, the coupling pin extending radially from an outer surface of the slot sleeve, coupling pin received and movable within the connecting sleeve guide slot for moving the slot sleeve between an unexpanded and an expanded position and for coupling the introducer assembly to a sheath assembly.

16. A method of expanding a sheath comprising:
advancing an elongated core member into a central lumen of an introducer body, wherein at least a portion of the introducer body is configured to expand from an unexpanded configuration in which the introducer body has a first outer diameter to an expanded configuration in which the introducer body has a second, larger, outer diameter due to an outwardly directed radial force exerted on the central lumen of introducer body by the core member as it moves between an unexpanded and expanded position within the central lumen;
coupling the core member to the introducer body;
positioning the core member in the unexpanded position within the central lumen of the introducer body;
advancing the coupled core member and introducer body into the central lumen of an expandable sheath;
coupling the introducer body to the sheath;
positioning the introducer body in an unexpanded position with respect to the sheath; and
advancing the core member within the central lumen of the introducer body such that the core member moves distally within the introducer body from the unexpanded position to the expanded position where the core member exerts an outwardly directed radial force on the central lumen expanding the introducer body and a corresponding portion of the sheath.

17. The method of claim 16, wherein the core member includes an elongated body portion and an expansion element extending radially from an outer surface of the body portion,
wherein, when the core member is in the expanded position, contact between the expansion element and the central lumen of the introducer body provides the outwardly directed radial force on the central lumen of the introducer body directing expansion of the introducer body.

18. The method of claim 17, wherein the central lumen of the introducer body includes a cavity for receiving the expansion element when the core member is in the unexpanded position, the cavity having a size and shape corresponding to the size and shape of the expansion element, the cavity having a diameter greater than a diameter of a main portion of the central lumen,
wherein positioning the core member in the unexpanded position within the central lumen of the introducer body includes positioning expansion element within the cavity such that the introducer body is not expanded,
wherein advancing the core member to the expanded position within the central lumen includes moving the core member such that the expansion element moves from the cavity and into the main portion of the central lumen causing the introducer body to expand.

19. The method of claim 16, further including:
expanding a distal opening of the sheath by positioning the core member in the expanded position and advancing the core member and the introducer body within the central lumen of the sheath such that an expanded portion of the introducer body is advanced through a distal opening of the sheath, where the core member exerts an outwardly directed radial force expanding the distal opening.

20. A method of delivering a medical device through a sheath, the method comprising:
providing a radially expandable sheath including a continuous inner layer defining a central lumen therethrough, the inner layer having at least one folded portion extending along a length of the inner layer;
advancing an elongated core member into the central lumen of an introducer body, at least a portion of the introducer body is configured to expand from an unexpanded configuration in which the introducer body has a first outer diameter to an expanded configuration in which the introducer body has a second, larger, outer diameter due to an outwardly directed radial force exerted on the central lumen of introducer body by the core member as it moves between an unexpanded and expanded position within the central lumen;
coupling the core member to the introducer body;
positioning the core member in the unexpanded position within the central lumen of the introducer body;
advancing the coupled core member and introducer body into central lumen of the sheath expandable sheath;
coupling the introducer body to the sheath;
positioning the introducer body in an unexpanded position with respect to the sheath;
inserting the introduce body and the sheath at least partially into the blood vessel of the patient;
advancing the core member within the central lumen of the introducer body such that the core member moves from the unexpanded position to the expanded position where the core member exerts an outwardly directed radial force on the central lumen expanding the introducer body and a corresponding portion of the sheath;
moving the introducer body within the central lumen of the sheath causing the sheath to locally expand from the unexpanded configuration to the expanded configuration at a location proximate the medical device in response to the outwardly directed radial force of the introducer body exerted against the inner layer and locally contracting the sheath at least partially back to the unexpanded configuration as the introducer body passes through the lumen;
withdrawing the introducer body from the sheath; and
advancing a medical device through the central lumen of the sheath and beyond the distal opening of the sheath to the treatment site.
